(19) United States
(12) Patent Application Publication
(10) Pub. No.: US 2023/0121245 A1
(43) Pub. Date: Apr. 20, 2023

(54) REGENERATIVE NONSTEROIDAL ANTI-INFLAMMATORY COMPOSITIONS, METHODS OF PRODUCTION, AND METHODS OF USE THEREOF

(71) Applicant: CROWN SCIENTIFIC, L.L.C., San Antonio, TX (US)

(72) Inventor: Ramon Coronado, San Antonio, TX (US)

(21) Appl. No.: 17/967,469

(22) Filed: Oct. 17, 2022

Related U.S. Application Data
(60) Provisional application No. 63/256,308, filed on Oct. 15, 2021.

(51) Int. Cl. C12N 5/073; G01N 33/50; A61P 29/00; A61K 35/51; A61K 45/06; A61P 37/06; A61P 9/00; A61P 17/00; A61P 19/04; A61P 11/00; A61P 27/02; A61P 35/02

(52) U.S. Cl. CPC C12N 5/0605; G01N 33/505; A61P 29/00; A61K 35/51; A61K 45/06; A61P 37/06; A61P 9/00; A61P 17/00; A61P 19/04; A61P 11/00; A61P 27/02; A61P 35/02; C12N 2509/00

(57) ABSTRACT

The disclosure provides nonsteroidal anti-inflammatory compositions and methods of use thereof. Specifically, the disclosure provides cell-free or substantially cell-free regenerative nonsteroidal anti-inflammatory compositions derived from placenta and/or from MSC cells isolated therefrom, methods for producing said compositions, and uses thereof to treat chronic and acute inflammatory conditions and diseases.

### RELATED APPLICATIONS

This application relates to and claims priority to U.S. Provisional Application No. 63/256,308, filed on Oct. 15, 2021, the contents of which are incorporated by reference in their entirety.

### FIELD OF THE ART

The present disclosure generally relates to nonsteroidal anti-inflammatory compositions, and more particularly, to cell-free or substantially cell-free regenerative nonsteroidal anti-inflammatory compositions derived from placenta and/or from MSC cells isolated therefrom, methods for producing said compositions, and uses thereof to treat chronic and acute inflammatory conditions and diseases.

### BACKGROUND

The World Health Organization ranks chronic inflammatory diseases as the greatest threat to human health. Worldwide, three out of five people die due to chronic inflammatory diseases. In the United States, 60% of people have one inflammatory disease or condition, 42% of people have more than one inflammatory disease or condition, and 12% of people have more than five inflammatory diseases or conditions.

Currently, inflammation is often treated with steroids as well as nonsteroidal anti-inflammatory drugs (NSAIDs). But both steroids and NSAIDs are cytotoxic and therefore inhibit healing and regeneration.

Stem cell therapy is an emerging therapeutic approach for treating inflammation. Although stem cells reduce inflammation and also promote healing, stem cell therapy has numerous hurdles. For example, protecting stem cell intellectual property and regulating stem cells for therapeutic commercial use remains ambiguous and highly complex. Further, living stem cells must remain frozen, which increases costs and complicates logistics for storage and distribution. Additionally, there are issues related to determining the appropriate dosage of live cells, especially considering that a portion of the cells may have died prior to administration to a patient. This problem is exacerbated by the fact that there is little to no validation of claims to having the best stem cell technology or the most living cells in a given product in today's saturated stem cell market.

### BRIEF SUMMARY

The present disclosure generally encompasses a cell-free or substantially cell-free regenerative nonsteroidal anti-inflammatory (RNSA) composition suitable for therapeutic or prophylactic use comprising a therapeutically or prophylactically effective amount of an isolated cell-free or substantially cell-free placenta-derived extract obtained from placental tissue from one or more mammalian donors wherein such tissue has naturally or been induced to undergo lysis, preferably stress-induced lysis, by a mechanism selected from cellular stress, apoptosis, necrosis, anoikis, or non-apoptotic programmed cell death, wherein said placenta-derived extract may comprise one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA, wherein said composition optionally is capable of inhibiting proliferation of activated T cells and/or is non-cytotoxic for one or more cells selected from stromal cells, mesenchymal stromal cells (MSCs), parenchymal cells, and tenocytes in a subject in need thereof, in vivo, or in vitro.

In some embodiments, the placenta may be selected from human, non-human primate, pig, sheep, horse, cow, dog, cat, rat, and mouse placenta. In some embodiments, the placenta may preferably be human placenta.

In some embodiments, the placental tissue may be obtained from a single donor.

In some embodiments, the placental tissue may be obtained from more than one donor (pooled donor placental tissue sample).

In some aspects, the placenta may comprise at least one placental tissue selected from amniotic membrane, chorion membrane, chorionic villus, umbilical cord, and Wharton's Jelly. The placenta may preferably be selected from at least one of amniotic membrane and/or chorion membrane.

In some embodiments, the at least one placental tissue may comprise perinatal stromal cells (PSCs) and/or mesenchymal stromal cells (MSCs).

In certain embodiments, the RNSA composition may be stable in solution at room temperature for at least eight weeks.

In certain embodiments, the RNSA composition may be stable to lyophilization.

In some embodiments, the RNSA composition may be capable of inhibiting proliferation of activated T cells, wherein the T cells are CD4+, CD8+, CD4+/CD8+, CD11c+, CD11b+, and/or CD56+ T cells.

In some embodiments, the composition may be further capable of promoting proliferation of one or more cells selected from stromal cells, mesenchymal stromal cells (MSCs), parenchymal cells, and tenocytes in a subject, in vivo, or in vitro.

In some embodiments, the RNSA composition may be capable of reducing expression of one or more pro-inflammatory cytokines from activated peripheral blood mononucleated cells (PBMCs) and/or activated T cells in a subject, in vivo, or in vitro. The one or more pro-inflammatory cytokines may be selected from TNFα, NFκB, IL-17A, IL-6, and IFNγ.

In some embodiments, the RNSA composition may be capable of increasing cAMP production from activated T cells in a subject, in vivo, or in vitro.

Moreover, the present disclosure also generally encompasses a method for producing a cell-free or substantially cell-free regenerative nonsteroidal anti-inflammatory (RNSA) composition. The method may comprise (i) obtaining at least one placental tissue from at least one mammal selected from human, non-human primate, pig, sheep, horse, cow, dog, cat, rat, and mouse, wherein the at least one placental tissue is selected from amniotic membrane, chorion membrane, chorionic villus, umbilical cord, and Wharton's Jelly, and wherein the at least one placental tissue comprises perinatal stromal cells (PSCs); (ii) optionally isolating the PSCs from said placental tissue and culturing the PSCs in at least one cell culture medium; (iii) permitting stress-induced lysis of said placental tissue and PSCs comprised therein and/or permitting stress-induced lysis of PSCs isolated therefrom to naturally occur and/or inducing stress-induced lysis of said placental tissue and PSCs comprised therein and/or inducing stress-induced lysis of PSCs isolated therefrom to produce a placenta-derived extract; and (iv) separating the placenta-derived extract or a portion thereof from the cells and tissue, for example, by decantation, centrifugation, and/or filtration; thereby producing the cell-free or substantially cell-free regenerative nonsteroidal anti-inflammatory composition.

In some embodiments, the PSCs may comprise mesenchymal stromal cells (MSCs).

In some embodiments, the mammal may be a human.

In some embodiments, the method may further comprise conducting one or more screening assays to assess the effects of the isolated placenta-derived extract or one or more portions thereof on the proliferation of activated T cells and/or the proliferation of one or more cells selected from stromal cells, mesenchymal stromal cells (MSCs), parenchymal cells, and tenocytes and/or on the expression of pro-inflammatory cytokines and/or the expression of anti-inflammatory cytokines in a mammalian subject or in vitro.

In some embodiments, different portions of the isolated placenta-derived extract may be screened in order to assess potency.

In some embodiments, inducing stress-induced lysis may comprise serum deprivation, nutrient deprivation, and/or hypoxia.

In some embodiments, inducing stress-induced lysis may comprise (i) contacting the placental tissue with a non-cell culture medium in a ratio ranging from about 1 mL non-cell culture medium per 1 gram of placental tissue to about 100 mL non-cell culture medium per 1 gram of placental tissue, preferably in a ratio of about 10 mL non-cell culture medium per 1 g of placental tissue; and (ii) incubating the placental tissue in the non-cell culture medium in an air-tight environment at a temperature ranging from about 4° C. to about 42° C., preferably at about 37° C., for about 2 days to about 12 days, preferably for about 10 days, wherein the incubating optionally comprises agitation, for example, at about 90 rpm.

In some aspects, the method may further comprise isolating the placental tissue PSCs and culturing the PSCs in at least one cell culture medium prior to inducing stress-induced lysis, optionally by nutrient deprivation and/or hypoxic conditions.

In some embodiments, inducing stress-induced lysis may comprise (i) replacing the at least one cell culture medium with a non-cell culture medium; and (ii) incubating the cultured MSCs in the non-cell culture medium in an air-tight environment at a temperature ranging from about 4° C. to about 42° C., preferably at about 37° C., for about 3 days to about 5 days, preferably for about 4 days, wherein the incubating optionally comprises agitation.

In some embodiments, the cultured PSCs may be cultured to at least 80% confluence.

In some embodiments, the non-cell culture medium may comprise saline solution.

In some embodiments, the saline solution may comprise 0.9% NaCl.

In some embodiments, the saline solution may comprise phosphate-buffered saline (PBS).

In some embodiments, the air-tight environment may prevent gas exchange, thereby inducing a hypoxic environment.

In some embodiments, the method may further comprise washing the placental tissue with phosphate-buffered saline (PBS) prior to inducing stress-induced lysis.

In some embodiments, the method may further comprise mincing the placental tissue prior to inducing stress-induced lysis.

In some embodiments, the method may further comprise washing the cultured MSCs with phosphate-buffered saline (PBS) prior to inducing stress-induced lysis.

In some embodiments, the method may further comprise contacting the placental tissue with one or more antimicrobial agents.

In some embodiments, the method may further comprise centrifugation at about 10,000×g for about 30 minutes.

In some embodiments, the method may further comprise filtration through a 0.45 μm membrane.

In some embodiments, the method may further comprise filtration through a 0.2 μm membrane, i.e. sterile filtration.

In some embodiments, the method may further comprise filtration through a 30 KDa MWCO membrane, a 10 KDa MWCO membrane, a 5 KDa MWCO membrane, a 3 KDa MWCO membrane, and/or a 2 KDa MWCO membrane.

Moreover, the present disclosure also generally relates to a cell-free or substantially cell-free regenerative nonsteroidal anti-inflammatory (RNSA) composition produced by the methods described herein.

In some embodiments, the composition may comprise one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA.

In some embodiments, the composition may be capable of inhibiting proliferation of activated T cells, wherein the T cells are CD4+, CD8+, CD4+/CD8+, CD11c+, CD11b+, and/or CD56+ T cells in a subject, in vivo, or in vitro.

In some embodiments, the composition may be non-cytotoxic for one or more cell types selected from stromal cells, mesenchymal stromal cells (MSCs), parenchymal cells, and tenocytes in a subject, in vivo, or in vitro.

In some embodiments, the composition may be capable of promoting proliferation of one or more cell types selected from stromal cells, mesenchymal stromal cells (MSCs), parenchymal cells, and tenocytes in a subject, in vivo, or in vitro.

In some embodiments, the composition may be capable of reducing expression of one or more pro-inflammatory cytokines from activated peripheral blood mononucleated cells (PBMCs) and/or activated T cells in a subject, in vivo, or in vitro.

In some embodiments, the one or more pro-inflammatory cytokines may be selected from TNFα, NFκB, IL17A, IL-6, and IFNγ.

In some embodiments, the composition may be capable of increasing cAMP production from activated T cells in a subject, in vivo, or in vitro.

In some embodiments, the composition may be stable in solution at room temperature for at least eight weeks.

In some embodiments, the composition may be stable to lyophilization.

The present disclosure also encompasses a method of treatment or prevention of at least one inflammatory condition or disease or at least one symptom associated therewith, comprising administering a therapeutically or prophylactically effective amount of the cell-free or substantially cell-free regenerative nonsteroidal anti-inflammatory (RNSA) composition of any one of the foregoing to a subject in need thereof.

In some embodiments, the at least one inflammatory condition or disease may be an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS.

In some embodiments, the at least one inflammatory condition or disease may be an acute inflammatory condition or disease optionally a viral infection associated with acute inflammation, further optionally a coronavirus infection, e.g., COVID-19 or SARS.

In some embodiments, the at least one inflammatory condition or disease may be selected from pneumonia, single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, inflammatory arthritis, gout, multiple sclerosis, psoriasis, Acute Respiratory Distress Syndrome (ARDS), wound healing, diabetic ulcers, non-healing wounds, lupus, and other autoimmune diseases associated with acute or chronic inflammation.

In some embodiments, the symptoms associated with the inflammatory condition may include one or more of pneumonia, cytokine storm, single or multiple organ failure, fibrosis, impaired respiratory function such as acute or chronic respiratory distress syndrome, fever, impaired cardiac function, impaired lung function, impaired liver function, impaired taste or smell, and impaired neurological function.

In exemplary embodiments, the subject may have pneumonia, optionally Covid-19-associated pneumonia and/or a pneumonia associated with another virus, e.g., influenza or another coronavirus, and/or a pneumonia associated with a fungus or bacterium.

In some embodiments, the ophthalmic inflammation may comprise one or more of corneal regeneration, corneal wound healing, corneal melting, dry eye, ocular infection, eyelid sty, and autoimmune-associated peripheral ulcerative keratitis.

In some embodiments, the fibrosis may comprise one or more of pulmonary fibrosis, cystic fibrosis, idiopathic pulmonary fibrosis, interstitial pulmonary fibrosis, radiation-induced lung injury, liver fibrosis, bridging fibrosis of the liver, cirrhosis, glial scar, arterial stiffness, arthrofibrosis, Crohn's disease, Dupuytren's contracture, keloid fibrosis, Mediastinal fibrosis, Myelofibrosis, Myocardial fibrosis, Peyronie's disease, Nephrogenic systemic fibrosis, Progressive massive fibrosis, pneumoconiosis, Retroperitoneal fibrosis, stromal fibrosis, Scleroderma, systemic sclerosis, Chronic obstructive pulmonary disease (COPD), asthma, and adhesive capsulitis.

In some embodiments, the gastrointestinal inflammation may comprise one or more of inflammatory bowel disease (IBD), Crohn's disease, ulcerative colitis, irritable bowel syndrome (IBS), and Celiac disease.

In some embodiments, the ophthalmic inflammation may be associated with keratoconjunctivitis sicca.

In some embodiments, the dermatologic inflammation may comprise eczema and psoriasis.

In some embodiments, the at least one autoimmune disease may be selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (UP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In some embodiments, the composition may be administered by one or more of injection, optionally intravenous (IV), subcutaneous (SC) administration, nebulization, and eye drops.

In exemplary embodiments, the effective amount may comprise one or more doses of the composition. In some embodiments, each dose may be selected from a list of dosage ranges comprising 0.01-5 mL, preferably 1 mL administered locally to locations such as tendons, ligaments, and joints; 0.01-2 mL, preferably 0.1 mL administered to each eye for topical eye indications; 5-100 mL, preferably 8 mL administered systemically; 0.5-5 mL, preferably 3 mL administered as an inhaled mist.

In some embodiments, the subject may be selected from a human, non-human primate, pig, sheep, horse, cow, dog, cat, rat, and mouse. In preferred embodiments, the subject may be human.

In some embodiments, the method of treatment or prevention may further comprise the administration of at least one other active, e.g., an anti-inflammatory agent such as an anti-inflammatory antibody or anti-inflammatory fusion protein, an antiviral agent, an antibacterial agent, an antifungal agent, an analgesic, an anti-congestive agent, an anti-fever agent, or a combination of any of the foregoing.

In exemplary embodiments, the subject may have been diagnosed with or is suspected of having a coronavirus infection, optionally COVID-19.

In some embodiments, the subject may have been diagnosed with a coronavirus infection, optionally COVID-19, and is on a respirator, has Acute Respiratory Distress Syndrome, and/or is experiencing respiratory difficulties.

In some embodiments, the subject may have been diagnosed with or suspected of having a coronavirus infection, optionally COVID-19, and optionally the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing.

In some embodiments, a method of treatment or prevention of at least one inflammatory or autoimmune condition or disease associated wherein TNFα is associated with the disease pathology or side effects of a treatment regimen, comprising administering a therapeutically or prophylactically effective amount of a cell-free or substantially cell-free regenerative nonsteroidal anti-inflammatory composition or a cell-free filtrate derived therefrom as afore described, optionally a fraction comprising molecules <1 KDa, to a subject in need thereof; optionally wherein said inflammatory or autoimmune condition or disease or side effects of a treatment regimen comprises one or more of rheumatoid arthritis, psoriatic arthritis, Crohn's disease, fatty liver disease, NASH, asthma, an inflammatory metabolic disorder, optionally type 1 or type 2 diabetes or obesity, inflammatory bowel disease, noninfectious uveitis, sepsis, cytokine storm, cancer, side effects associated with cancer therapy, optionally radiotherapy, chemotherapy, hormone and/or biologic therapy, side effects associated with transplanted cells, tissues and/or or organ, optionally bone marrow transplant, inflammation associated with an acute or chronic viral condition, a neuroinflammatory condition, optionally multiple sclerosis, Alzheimer's disease, migraine, Neuromyelitis optica (NMO), Anti-myelin oligodendrocyte glycoprotein antibody disorder (MOG), Autoimmune encephalitis, Transverse Myelitis, Optic neuritis, neurosarcoidosis, Parkinson's disease, or schizophrenia; further optionally wherein TNFα levels are detected in the treated subject before, during and/or after treatment; still further optionally wherein treatment is only effected if TNFα levels are elevated in the subject prior to treatment and/or treatment efficacy is monitored after treatment based on the [reducing] effect of the treatment on TNFα levels.

In some embodiments, the invention provides a method of activating a Peroxisome proliferator-activated receptors (PPARs), optionally PPARα, PPARβ/δ or (PPARδ), and/or PPARγ in a subject in need thereof by administering a therapeutically or prophylactically effective amount of a cell-free or substantially cell-free regenerative nonsteroidal anti-inflammatory composition or a cell-free filtrate derived therefrom as afore-described, optionally a fraction comprising molecules <1 KDa; optionally wherein the subject has a condition associated with reduced or inhibited activities associated with the activation of Peroxisome proliferator-activated receptors (PPARs), optionally PPARα, PPARβ/δ or (PPARδ), and/or PPARγ, further optionally wherein activities elicited by Peroxisome proliferator-activated receptors (PPARs), optionally PPARα, PPARβ/δ or (PPARδ), and/or PPARγ are detected in a sample from the subject before, during and/or after treatment, still further optionally wherein the subject has a metabolic disorder, hypertrophic obesity, insulin-resistance, acute or chronic inflammatory kidney disease, cancer, non-alcoholic fatty liver disease (NAFLD), non-alcoholic steatohepatitis (NASH), fibrosis, Alzheimer's disease (AD), dyslipidemia, hyperglycemia, type 1 or 2 diabetes, a lung disease such as COPD or asthma, a neurodegenerative disorder, obesity, hypertension, atherosclerosis, a cardiovascular condition, vascular injury, heart attack, myocarditis, pericarditis, contractile dysfunction, stroke, hypertension, a neurodegenerative condition, diabetic nephropathy, retinopathy, inflammatory bowel disease, ulcerative colitis, or Crohn's disease (CD).

In some embodiments, the invention provides a method of treatment or prevention of at least one condition or disease wherein reactive oxygen species are associated with the disease pathology or wherein reactive oxygen species are side effects associated with a treatment regimen, optionally chemotherapy, radiotherapy, gene therapy, cell therapy, cell, organ and/or tissue transplantation, comprising administering a therapeutically or prophylactically effective amount of a cell-free or substantially cell-free regenerative nonsteroidal anti-inflammatory composition or a cell-free filtrate derived therefrom, optionally a fraction comprising molecules <1 KDa, to a subject in need thereof; optionally wherein the condition or disease comprises a respiratory condition such as asthma, chronic obstructive pulmonary diseases, inflammatory bowel disease, neurodegenerative disorders such as Alzheimer's disease (AD), Huntington's disease (HD), amyotrophic lateral sclerosis (ALS), Parkinson's disease (PD), and spinocerebellar ataxia (SCA), cardiovascular diseases such as atherosclerosis, cardiac hypertrophy, ischemic-reperfusion injury, myocyte apoptosis, heart failure, cancers, optionally lung, breast, tongue, gastric, larynx, colon, rectal, lung and prostate cancers; further optionally cancers wherein ROS contribute to resistance to therapies, metastasis, aberrant angiogenesis and normal or aberrant aging.

In some embodiments, the invention provides a method of reducing T cell proliferation in a subject in need thereof, by administering a therapeutically or prophylactically effective amount of a cell-free or substantially cell-free regenerative nonsteroidal anti-inflammatory composition or a cell-free filtrate derived therefrom, optionally a fraction comprising molecules <1 KDa; optionally wherein the subject has an inflammatory or autoimmune condition wherein T cells contribute to the disease pathology; further optionally wherein the condition comprises rheumatoid arthritis (RA), systemic lupus erythematosus (SLE), ITP, CD, IBD, myositis, psoriasis, psoriatic arthritis, vasculitis, scleroderma, type 1 or type 2 diabetes, hypothyroidism, Addison's disease, sepsis, cytokine storm, a neurodegenerative condition, neurological dysfunction or impairment, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, asthma, glomerulonephritis, pancreatitis, hepatitis, inflammatory arthritis, gout, multiple sclerosis, Acute Respiratory Distress Syndrome (ARDS), wound healing, diabetic ulcers, non-healing wounds, lupus, or other autoimmune diseases associated with T cell mediated acute or chronic inflammation; further optionally wherein T cell proliferation and/or T cell activation is detected in the subject or samples from the subject prior, during or after treatment.

### DESCRIPTION OF THE DRAWINGS

FIG. 1A illustrates an exemplary method for preparing human placenta for isolation of amniotic membrane.

FIG. 1B illustrates an exemplary method for peeling or removing amniotic membrane from chorion.

FIG. 1C illustrates an exemplary method for washing amniotic membrane with PBS.

FIG. 2 presents data showing normalized activated T cell proliferation as a model for inflammation and inhibition of the T cell proliferation of greater than 85% by MSCs (positive control). Results for cell-free extracts prepared from nineteen conditions (C1-C19, various tissues and conditions for treating those tissues) are also shown. C14 represents the cell-free regenerative nonsteroidal anti-inflammatory composition (RNSA) described in Example 1.

FIG. 3 presents data showing normalized activated T cell proliferation as a model for inflammation. It can be seen therefrom that the cell-free regenerative nonsteroidal anti-inflammatory composition described in Example 1 (RNSA), inhibited T cell proliferation by greater than 85%, comparable to the T cell proliferation inhibition achieved by MSCs, whereas the control, (commercial cell-free reagent) did not inhibit activated T cell proliferation.

FIG. 4 presents data showing activated T cell proliferation as a model for inflammation and results from testing different methods of cell death on the potency of the cell-free RNSA composition.

FIG. 5 presents data showing activated T cell proliferation as a model for inflammation and results from testing various filtration membranes on the potency of the cell-free RNSA composition.

FIG. 6 presents data showing activated T cell proliferation as a model for inflammation and results from testing the effect of lyophilization on the potency of the RNSA compositions.

FIG. 7 presents data showing activated T cell proliferation as a model for inflammation and results from testing the effect of DNase, RNase, and Proteinase K on the potency of the RNSA composition produced from amniotic membrane tissue extraction described in Example 1.

FIG. 8 presents data showing activated T cell proliferation as a model for inflammation and results from testing the effect of DNase, RNase, and Proteinase K on the potency of the RNSA composition produced from cultured hMSCs described in Example 2.

FIG. 9 presents data showing activated T cell viability as a model for inflammation and results from testing the effect of DNase, RNase, and Proteinase K on the potency of the RNSA composition produced from amniotic membrane tissue extraction described in Example 1.

FIG. 10 presents data showing activated T cell proliferation as a model for inflammation and results from testing the effect of time during extraction and shaking vs. non-shaking conditions on the potency of the RNSA composition produced from amniotic membrane tissue extraction described in Example 1.

FIG. 11 presents data showing activated T cell proliferation as a model for inflammation and results from testing the effect of tissue type and culture media on the potency of the RNSA composition produced from tissue extractions described in Example 1.

FIG. 12 presents data showing activated T cell proliferation as a model for inflammation and results from testing the stability of the RNSA compositions stored at room temperature (RT) and at 4° C. for two months.

FIG. 13 presents data showing activated T cell proliferation as a model for inflammation and results from testing the effect of EP Receptor blockers on the potency of the RNSA compositions.

FIG. 14 presents data showing activated CD4+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 15 presents data showing activated CD8+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 16 presents data showing activated CD4+/CD8+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 17 presents data showing activated CD11c+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 18 presents data showing activated CD11b+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 19 presents data showing activated CD56+ T cell proliferation within a PBMC sample as a model for inflammation and results from testing the effect of the tissue extraction process described in Example 1 (“AM”) compared to the cultured MSCs extraction process described in Example 2 (“BR”) on the potency of the RNSA compositions.

FIG. 20 shows that the RNSA composition reduces the expression level of TNFα from activated PBMCs.

FIG. 21 shows that the RNSA composition reduces the expression level of NFκB from activated PBMCs.

FIG. 22 shows that the RNSA composition reduces the expression level of IL-17A from activated PBMCs.

FIG. 23 shows that the RNSA composition reduces the expression level of IFNγ from activated PBMCs.

FIG. 24 shows that the RNSA composition promotes/induces cAMP production by activated T cells.

FIG. 25 shows results of the eicosanoid analysis of RNSA compositions.

FIG. 26 shows a list of eicosanoids which were not detected in the eicosanoid analysis of RNSA compositions.

FIG. 27 shows proliferation of human stromal cells as a model for regeneration and promotion of stromal cell proliferation by the RNSA composition. In contrast, the steroid is completely cytotoxic.

FIG. 28 shows proliferation of hMSCs as a model for regeneration and promotion of hMSC proliferation by the RNSA composition. In contrast, the steroid is completely cytotoxic.

FIG. 29 shows proliferation of human parenchymal cells as a model for regeneration and promotion of human parenchymal cells proliferation by the RNSA composition. In contrast, the tested commercial compounds were each cytotoxic to varying degrees.

FIG. 30 shows proliferation of human tenocytes as a model for regeneration and promotion of human tenocytes proliferation by the RNSA composition. In contrast, the tested commercial compounds were each cytotoxic to varying degrees.

FIG. 31 shows data relating to survival proportions for a mouse model of Graft versus

Host Disease.

FIG. 32 is a plot of the GvHD score versus normalized days for the GvHD mice injected with media (PBMC) or with an exemplary cell-free regenerative nonsteroidal anti-inflammatory composition (Cell-Free) according to the invention.

FIG. 33 is a plot of the body weight GvHD mice injected with media (PBMC) or with an exemplary cell-free regenerative nonsteroidal anti-inflammatory composition (CM) according to the invention.

FIG. 34A is a photo of GvHD mouse model kidneys.

FIG. 34B is a plot of the GvHD mouse kidney areas in mm².

FIG. 35 is a photo of the GvHD mice. A GvHD mouse treated with an exemplary RNSA composition according to the invention is shown on the left whereas an untreated GvHD mouse is shown on the right.

FIG. 36 shows plots quantifying the levels of inflammatory markers IL-17 and IFNγ at Day 21 and Day 42 in a mouse model of cardiomyopathy.

FIG. 37 shows plots quantifying the inflammatory markers histopathology disease scores (H&E) and the Trichrome fibrosis scores at Day 21 in a mouse model of cardiomyopathy.

FIG. 38 shows a photo of a human patient having eczema on the left hand on Day 1 and a photo of the same on Day 60 after a first subcutaneous injection of an exemplary RNSA composition according to the invention on Day 1 and a second subcutaneous injection of the same RNSA composition on Day 30.

FIG. 39 shows an ultrasound image of a human ankle tendinosis prior to treatment (subcutaneous injection of an exemplary RNSA composition according to the invention at the site of tendinosis) and an ultrasound image of the healed tendon 30 days after treatment.

FIG. 40 shows two photos of a human patient having an eyelid sty prior to treatment with eye drops comprising an exemplary RNSA composition according to the invention (top) and two photos of the same eye six weeks after treatment showing that the sty had completely healed (bottom).

FIG. 41 shows a graph of total free fatty acid (FFA) content from five vials of manufactured samples of RNSA composition according to Example 10.

FIG. 42A shows a graph of resting pain scores from human patients having tendinopathy of both elbows after injection of the inventive RNSA composition according to Example 11.

FIG. 42B shows a graph of active pain scores from human patients having tendinopathy of both elbows after injection of the inventive RNSA composition according to Example 11.

FIG. 43A shows a results of Eicosanoid/Oxylipin Biomarker Profiling of human plasma following injection of the inventive RNSA composition according to Example 12.

FIG. 43B shows a graph of total FFA content in human plasma following injection of the inventive RNSA composition according to Example 12.

FIG. 43C shows a graph of total FFA content in human plasma determined by LabCorp following injection of the inventive RNSA composition according to Example 12.

FIG. 43D shows a graph of biomarker 15dPGJ2 content in human plasma following injection of the inventive RNSA composition according to Example 12.

FIG. 43E shows a graph of biomarker 18-HEPE content in human plasma following injection of the inventive RNSA composition according to Example 12.

FIG. 43F shows a graph of biomarker 18-HETE content in human plasma following injection of the inventive RNSA composition according to Example 12.

FIG. 44A shows a graph of inhibition of percent proliferation of T cells by the inventive RNSA composition following long-term storage according to Example 13.

FIG. 44B shows a graph of percent viability of T-effector cells after treatment with the inventive RNSA composition following long-term storage according to Example 13.

FIG. 45 shows a graphs of regulation of human T-effector cells (e.g., CD4 and CD8 cells) by the inventive RNSA composition according to Example 14.

FIG. 46A-D contains the results of experiments in the PMBC monocyte/macrophage model experiment demonstrating that RNSA inhibits LPS induced TNFα secretion in LPS stimulated PBMCs. In the experiments TNFα secretion and percent of TNFα inhibition were detected after no treatment after 24 hours (negative control) in comparison to co-treatment with 100 ng LPS and RNSA (GMP Lots 101421B, 082721A, 100821A) (A, C); and in comparison to co-treatment with 100 ng LPS and Betamethasone (0.13% DMSO final concentration) (B, D). n=2-3/treatment. a-technical outliers, not represented in C.

FIGS. 47A and B contain the results of experiments demonstrating the effects of different RNSA lots (RNSA1 (082721A) and RNSA2 (100821A)) on PPARα and PPARγ. As shown in the Figure both RNSA1 and RNSA2 exhibited dose-dependent stimulation of PPARα and PPARγ reporter activities (Single Factor ANOVA with p0.3). The actual effects by the RNSA samples are even larger than shown in the Figure if corrected for the effect of saline vehicle controls above. Stimulatory effects of up to near 50% of maximal responses in PPARα and PPARγ reporter activation are revealed after such correction.

FIG. 48 contains experimental results demonstrating the preventative effects of RNSA & RNSA filtrates on reactive oxygen species induced cell death in Friedreich's Ataxia fibroblasts. The results show that RNSA partially rescues ROS-induced death in healthy fibroblasts and Friedreich's Ataxia patients. RNSA Cytoprotective/anti-ROS effects can be seen in <1 KDa fractions.

FIG. 49A-D shows the development of a T cell proliferation assay used to assess the potency of RNSA lots on acute lymphoblastic leukemia cell lines Jurkat and CCRF-CEM. In the experiments Jurkat and CCRF-CEM T cell lines were utilized to assess the effect of RNSA lots (082721, 100821 and 101421) on inhibition of cellular proliferation (A) and percent viability (B) after 48 hours of treatment with the drug candidate compared to saline. Optimization of the T cell proliferation assay with the CCRF-CEM T lymphoblasts after culturing for 24 and 48 hours was assessed by fold change in proliferation compared to 24-hour timepoint of no treatment group (C) and percent viability (D). Statistical analysis represented by Two-way ANOVA with Šidák's multiple comparisons using a post-hoc test was effected where &<0.05 represents comparison to saline (A, B); a<0.05 against NT, & <0.05 against saline 24 hrs, $<0.05 against saline at 48 hours, and *<0.05 24 vs 48 hours. n=2-3. NT-no treatment/standard media, saline-0.9% Sodium Chloride, RNSA lots-082721, 100821, 101421.

FIGS. 50A & 50B: Assessment of RNSA potency of different RNSA lots. In the experiments RNSA lots 051322A, 051322B, 051322C, 050622A and 051022A were assessed for inhibition of CCCRF-CEM T cell proliferation (A) and viability (B) as per T cell assay described above. A one-way ANOVA with post-hoc Welch's test was performed, *p<0.05 against saline.

### DETAILED DESCRIPTION

### I. Overview

Provided herein are cell-free (or substantially cell-free) regenerative nonsteroidal anti-inflammatory compositions derived from placenta, methods for producing said compositions, and uses thereof to treat chronic and acute inflammatory conditions and diseases.

It is contemplated that any embodiment discussed in this specification can be implemented with respect to any method, kit, reagent, or composition of the disclosure, and vice versa. Furthermore, compositions of this disclosure can be used to achieve methods of the disclosure.

It will be understood that particular embodiments described herein are shown by way of illustration and not as limitations. The principal features of this disclosure can be employed in various embodiments without departing from the scope of the disclosure. Those skilled in the art will recognize, or be able to ascertain, using no more than routine experimentation, numerous equivalents to the specific procedures described herein. Such equivalents are considered to be within the scope of this disclosure and are covered by the appended claims.

All publications and patent applications mentioned in the instant specification are indicative of the level of skill of one skilled in the art to which this disclosure pertains. All publications and patent applications are herein incorporated by reference to the same extent as if each individual publication or patent application was specifically and individually indicated to be incorporated by reference.

Unless defined otherwise, all technical and scientific terms used herein have the same meaning as is commonly understood by one of skill in the art to which this disclosure belongs. In the event that there are a plurality of definitions for terms herein, those in this section prevail. Where reference is made to a URL or other such identifier or address, it is to be understood that such identifiers can change and particular information on the internet can come and go, but equivalent information can be found by searching the internet. Reference thereto evidences the availability and public dissemination of such information.

As used herein, the singular forms “a,” “an,” and “the” may mean “one” but also include plural referents such as “one or more” and “at least one” unless the context clearly dictates otherwise. All technical and scientific terms used herein have the same meaning as commonly understood to one of ordinary skill in the art to which this invention belongs unless clearly indicated otherwise.

As used herein, the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the inherent variation of error for the device, the method being employed to determine the value, or the variation that exists among the study subjects.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 8, 9, 10, 11, 12, 13, 14, or 15%.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, “apoptosis” refers to programmed cell death via a highly-regulated, genetically-directed process of cell self-destruction that is marked by the fragmentation of nuclear DNA, is activated either by the presence of a stimulus or removal of a suppressing agent or stimulus and is a normal physiological process.

As used herein, “non-apoptotic programmed cell death” refers to programmed cell death by a mechanism that is caspase independent.

As used herein, “stress-induced lysis” refers to any mechanism of cell lysis induced by a wide range of molecular or biomolecular stimuli that induce a stress response in the cell at the biomolecular level followed by damage to the cellular structure which results in release of intracellular material. Stress-induced lysis my occur by any number of mechanisms including cellular stress, apoptosis, necrosis, anoikis, necroptosis, eryptosis, aponecrosis, paroptosis, pyropotosis, or other form of non-apoptotic programmed cell death

As used herein “cell-free” generally refers to a composition or extract, e.g., a placental derived extract, wherein all cells originally contained in the composition or extract have been removed or rendered non-viable. In the present invention this is generally achieved by inducing stress-induced lysis such as by use of nutrient deprivation and removal of live and/or apoptosed cells such as by the use of decantation, centrifugation, and/or filtration.

As used herein “substantially cell-free” generally refers to a composition or extract, e.g., a placental derived extract, wherein the majority of the cells originally contained in the composition or extract have been removed or rendered non-viable, e.g., wherein at least 70, 80, 90, 95, 99, 99.5, or 99.9% of the cells have been removed or rendered non-viable. In the present invention this is generally achieved by inducing stress-induced lysis such as by the use of nutrient deprivation and removal of live and/or apoptosed cells such as by the use of decantation, centrifugation, and/or filtration.

As used herein “non-cell culture medium” generally refers to a medium wherein cells are cultured that lacks cells and which moreover may lack nutrients which may induce nutrient deprivation, stress-induced lysis, apoptosis, and/or hypoxia of cells contained therein, e.g., a saline medium or composition.

As used herein, “treatment” (and grammatical variations thereof such as “treat” or “treating”) or “prevention” (and grammatical variations thereof such as “prevent” or “preventing”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result alone or in combination with other active agents.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. Generally, the response is either amelioration of symptoms in a patient or a desired biological outcome (e.g., reduction of tissue fibrosis, reduction of tissue inflammation, increase of immune modulation). The therapeutically effective amount may vary according to factors such as the disease state, age, sex, and weight of the subject. In some embodiments, the provided methods involve administering the compositions at effective amounts, e.g., therapeutically effective amounts alone or in combination with other active agents or therapies.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount will be less than the therapeutically effective amount. In the context of lower disease burden, the prophylactically effective amount in some aspects will be higher than the therapeutically effective amount.

By “pharmaceutically acceptable” it is meant that the carrier, diluent or excipient must be compatible with the other ingredients of the formulation and not deleterious to the recipient thereof. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid, vitamin A, vitamin E, and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, cysteine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG); retinyl palmitate, selenium, methionine, citric acid, sodium sulfate and parabens Examples of diluent include, but are not limited to, water, alcohol, saline solution, glycol, mineral oil and dimethyl sulfoxide (DMSO).

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation. The pharmaceutical composition may further contain additional pharmaceutical or therapeutic agent, as evaluated beneficial by the physician administering said pharmaceutical composition.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and non-human primates (including monkeys, chimpanzees, orangutans and gorillas) are included within the definition of subject.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well as infusion, inhalation, and nebulization. The phrases “parenteral administration” and “administered parenterally” as used herein means modes of administration other than enteral and topical administration.

### II. Regenerative Nonsteroidal Anti-Inflammatory (RNSA) Compositions

The present disclosure provides a cell-free or substantially cell-free regenerative nonsteroidal anti-inflammatory (RNSA) composition suitable for therapeutic or prophylactic use comprising an isolatable cell-free or substantially cell-free placenta-derived extract obtained from placental tissue from one or more mammalian donors, wherein said extract may comprise one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA. The composition may be capable of inhibiting proliferation of activated T cells and may further be non-cytotoxic for one or more cells selected from stromal cells, mesenchymal stromal cells (MSCs), parenchymal cells, and tenocytes in a subject, in vivo, or in vitro.

The cell-free or substantially cell-free regenerative nonsteroidal anti-inflammatory (RNSA) compositions derived from placenta provided herein may be specifically derived from placental “perinatal stromal cells” (PSCs), also referred to herein as mesenchymal stromal cells (MSCs) or which may comprise MSCs. More specifically, the cell-free or substantially cell-free regenerative nonsteroidal anti-inflammatory compositions may be derived from placental MSCs which have undergone lysis, preferably stress-induced lysis, by a mechanism selected from cellular stress, apoptosis, necrosis, anoikis, or non-apoptotic programmed cell death, e.g., naturally and/or by inducing apoptosis or other form of stress-induced lysis by exogenous means, optionally by the use of one or more of the methods disclosed or exemplified herein or other means known in the art for inducing cell apoptosis or other form of stress-induced lysis. It is also contemplated herein that the cell-free or substantially cell-free regenerative nonsteroidal anti-inflammatory (RNSA) compositions described herein may be derived from non-placental MSCs obtained from, e.g., bone marrow, adipose tissue, muscle, corneal stroma, and/or deciduous teeth dental pulp, i.e., “adult” MSCs.

As used herein, “perinatal stromal cell,” or “PSC” refers to cells isolated from a placenta. The placenta may be a human placenta or may be derived from any other mammal such as a non-human primate, a pig, a sheep, a horse, a cow, a dog, a cat, a rat, or a mouse. The placenta may preferably be a human placenta. The human placenta includes an umbilical cord, an amniotic membrane (amnion), and a “placenta proper”, which includes the chorion or chorionic plate, the villus, the intervillous space, the basal plate and the cotyledon. Each portion of the placenta can be isolated and can be used to derive subpopulations of perinatal stromal cells.

In some embodiments, the placental tissue may be obtained from a single donor. In some embodiments, the placental tissue may be obtained from more than one donor (pooled donor placental tissue sample).

In one embodiment, the placenta may comprise at least one placental tissue selected from amniotic membrane, chorion membrane, chorionic villus, umbilical cord, and Wharton's Jelly. In preferred embodiments, the placenta may comprise at least one placental tissue selected from amniotic membrane and/or chorion membrane.

The amnion membrane can be mechanically separated from the chorion, which leads to the derivation of amnion perinatal stromal cell (APSC). When sectioned longitudinally, the umbilical cord exposes Wharton's jelly, containing umbilical arteries and vein. After removal of the blood vessels, Wharton's Jelly perinatal stromal cell (WPSC, WJPSC, or MJ-MSC) can be derived from the umbilical cord. When the amnion and the umbilical cord are removed, the remaining portion of the placenta, which can be referred to as the placenta proper, can be used directly to prepare placenta proper stromal cell (PPSC), or can be further separated. For example, the chorionic membrane can be detached to isolate whole chorion derived stromal cell (CSC), and the intermediate and terminal villi can be exposed to isolate chorionic-villi stromal cell (CVC).

In some embodiments, the at least one placental tissue may comprise perinatal stromal cells (PSCs) and/or mesenchymal stromal cells (MSCs).

In certain embodiments, the RNSA composition may be stable in solution at room temperature for a prolonged time, e.g., at least 1, 2, 3, 4, 5, 6, 7 or at least 8 weeks or more.

In certain embodiments, the RNSA composition may be stable to lyophilization.

In some embodiments, the RNSA composition has been stored in a sealed vial at a temperature ranging from 4° C. to 40° C. for a time period ranging from 1 week to 24 weeks to enhance bioactivity, efficacy, and/or potency of the composition prior to administration to a patient in need thereof.

In some embodiments, the RNSA composition may elicit an anti-inflammatory response. As such, the RNSA composition may be capable of inhibiting proliferation of activated T cells in a subject, in vivo, or in vitro, wherein the T cells are CD4+, CD8+, CD4+/CD8+, CD11c+, CD11b+, and/or CD56+ T cells.

In some embodiments, the RNSA composition may be capable of promoting proliferation of one or more cells selected from stromal cells, mesenchymal stromal cells (MSCs), parenchymal cells, and tenocytes in a subject, in vivo, or in vitro.

Additionally, in some embodiments, the RNSA composition may be capable of reducing expression of one or more pro-inflammatory cytokines from activated peripheral blood mononucleated cells (PBMCs) and/or activated T cells and/or of promoting the expression or activity of one or more anti-inflammatory cytokines in a subject, in vivo, or in vitro. The one or more pro-inflammatory cytokines may be selected from TNFα, NFκB, IL17A, IL-6, and IFNγ.

And in some embodiments, the RNSA composition may be capable of increasing cAMP production from activated T cells in a subject, in vivo, or in vitro.

### III. Methods for Producing Regenerative Nonsteroidal Anti-Inflammatory Compositions

The present disclosure also generally encompasses a method for producing a cell-free or substantially cell-free regenerative nonsteroidal anti-inflammatory (RNSA) composition. The method may comprise (i) obtaining at least one placental tissue from a mammal selected from human, non-human primate, pig, sheep, horse, cow, dog, cat, rat, and mouse, wherein the at least one placental tissue is selected from amniotic membrane, chorion membrane, chorionic villus, umbilical cord, and Wharton's Jelly, and wherein the at least one placental tissue comprises perinatal stromal cells (PSCs); (ii) optionally isolating the PSCs from said placental tissue and culturing the PSCs in at least one cell culture medium (iii) permitting stress-induced lysis of said placental tissue and PSCs comprised therein and/or permitting stress-induced lysis of PSCs isolated therefrom to occur naturally and/or inducing or enhancing stress-induced lysis of said placental tissue and PSCs and/or inducing stress-induced lysis of PSCs isolated therefrom to produce an extract; and (iv) separating the extract or a portion thereof from the cells and tissue, for example, by decantation, centrifugation, and/or filtration; thereby producing the cell-free or substantially cell-free regenerative nonsteroidal anti-inflammatory composition and (v) optionally further purifying or concentrating said extract or the actives comprised therein, e.g., by concentrating the amount of one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA, in order to enhance its anti-inflammatory potency. In some embodiments, the PSCs may comprise MSCs. In some embodiments, the mammal may be a human.

In some embodiments, the method may further comprise conducting one or more screening assays to assess the effects of the isolated placenta-derived extract or one or more portions thereof on the proliferation of activated T cells and/or the proliferation of one or more cells selected from stromal cells, mesenchymal stromal cells (MSCs), parenchymal cells, and tenocytes and/or on the expression of pro-inflammatory cytokines and/or the expression of anti-inflammatory cytokines in a mammalian subject, in vivo, or in vitro.

In some embodiments, different portions of the isolated placenta-derived extract may be screened in order to assess potency.

In some embodiments, inducing stress-induced lysis may comprise serum deprivation, nutrient deprivation, and/or hypoxia.

In exemplary embodiments, inducing stress-induced lysis may comprise (i) contacting the placental tissue with a non-cell culture medium in a ratio ranging from about 1 mL non-cell culture medium per 1 gram of placental tissue to about 100 mL non-cell culture medium per 1 gram of placental tissue, preferably in a ratio of about 10 mL non-cell culture medium per 1 g of placental tissue; and (ii) incubating the placental tissue in the non-cell culture medium in an air-tight environment at a temperature ranging from about 4° C. to about 42° C., preferably at about 37° C., for about 2 days to about 12 days, preferably for about 10 days, wherein the incubating optionally comprises agitation, for example, at about 90 rpm. However, as afore-mentioned stress-induced lysis may alternatively be induced or enhanced by other methods known in the art for initiating or promoting stress-induced lysis.

In some embodiments, the method may further comprise washing the placental tissue with phosphate-buffered saline (PBS) prior to inducing stress-induced lysis. In some embodiments, the method may further comprise mincing the placental tissue prior to inducing stress-induced lysis. In some embodiments, the method may further comprise contacting the placental tissue with one or more antimicrobial agents.

In some embodiments, the non-cell culture medium may comprise saline solution. In some embodiments, the saline solution may comprise 0.9% NaCl. In some embodiments, the saline solution may comprise phosphate-buffered saline (PBS). In some embodiments, the air-tight environment may prevent gas exchange, thereby inducing a hypoxic environment.

In some aspects, the method may further comprise isolating the placental tissue PSCs and culturing the PSCs in at least one cell culture medium prior to inducing stress-induced lysis. In some embodiments, inducing stress-induced lysis may comprise (i) replacing the at least one cell culture medium with a non-cell culture medium; and (ii) incubating the cultured PSCs in the non-cell culture medium in an air-tight environment at a temperature ranging from about 4° C. to about 42° C., preferably at about 37° C., for about 3 days to about 5 days, preferably for about 4 days, wherein the incubating optionally comprises agitation. In some embodiments, the cultured PSCs may be cultured to at least 80% confluence.

In some embodiments, the non-cell culture medium may comprise saline solution. In some embodiments, the saline solution may comprise 0.9% NaCl. In some embodiments, the saline solution may comprise phosphate-buffered saline (PBS). In some embodiments, the air-tight environment may prevent gas exchange, thereby inducing a hypoxic environment.

In some embodiments, the method may further comprise washing the cultured PSCs with phosphate-buffered saline (PBS) prior to inducing stress-induced lysis.

In some embodiments, the method may further comprise centrifugation at about 10,000×g for about 30 minutes.

In some embodiments, the method may further comprise filtration through a 0.45 μm membrane. In some embodiments, the method may further comprise filtration through a 0.2 μm membrane, i.e. sterile filtration. In some embodiments, the method may further comprise filtration through a 30 KDa MWCO membrane, a 10 KDa MWCO membrane, a 5 KDa MWCO membrane, a 3 KDa MWCO membrane, and/or a 2 KDa MWCO membrane.

The present disclosure also generally relates to a cell-free or substantially cell-free regenerative nonsteroidal anti-inflammatory (RNSA) composition produced by any of the methods disclosed herein.

### IV. Methods for Treating Inflammatory Diseases and Conditions

In a further embodiment, a method of treatment of at least one inflammatory condition or disease or at least one symptom associated therewith is provided. The method may comprise administering a therapeutically or prophylactically effective amount of the cell-free or substantially cell-free regenerative nonsteroidal anti-inflammatory (RNSA) composition described herein to a subject in need thereof wherein such treatment optionally may reduce or prevent tissue inflammation in the subject.

In one aspect, a molecular marker of inflammation in the tissue is decreased as compared to said molecular marker in the tissue before the administration of the RNSA composition. In some aspects, the molecular marker of inflammation is selected from the group consisting of TNFα expression, NFκB expression, INFγ expression, IL-17 (or IL-17A) expression, IL-6 expression, and a combination thereof.

In some embodiments, the at least one inflammatory condition or disease may be an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS. In some embodiments, the at least one inflammatory condition or disease may be an acute inflammatory condition or disease, e.g., an acute inflammatory autoimmune condition or infectious condition associated with acute inflammation such as a viral condition associated with acute inflammation, further optionally a coronavirus infection, e.g., COVID-19 or SARS.

In some embodiments, the at least one inflammatory condition or disease or symptom associated therewith may be selected from pneumonia, single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, inflammatory arthritis, gout, multiple sclerosis, psoriasis, Acute Respiratory Distress Syndrome (ARDS), wound healing, diabetic ulcers, non-healing wounds, lupus, and at least one autoimmune disease associated with acute or chronic inflammation.

In some embodiments, the at least one inflammatory condition or disease may be pneumonia, e.g., caused by at least one virus, fungus, bacterium or a combination thereof. In exemplary embodiments, the pneumonia may be Covid-19-associated and/or influenza-associated pneumonia. Coronavirus Disease 2019 or Covid-19 is caused by severe acute respiratory syndrome coronavirus 2 (SARS-CoV-2 coronavirus). The predominant CT findings of Covid-19-associated pneumonia patients include conspicuous ground-glass opacification, consolidation, bilateral involvement, and peripheral and diffuse distribution.

In some embodiments, the at least one inflammatory condition or disease may comprise COVID-19 or other inflammatory condition or infection and the treatment or prevention may further comprise the administration of at least one other active, e.g., an anti-inflammatory agent such as an anti-inflammatory antibody or anti-inflammatory fusion protein, e.g., Embrel (etanercept), Humira (adalimumab), or an IL-6 antagonist, an antiviral agent, an antibacterial agent, an antifungal agent, an analgesic, an anti-congestive agent, an anti-fever agent, or a combination of any of the foregoing.

In some embodiments, the treated subject has been diagnosed with or is suspected of having a coronavirus infection, optionally COVID-19.

In some embodiments, the treated subject has been diagnosed with a coronavirus infection, optionally COVID-19, and is on a respirator, has Acute Respiratory Distress Syndrome (ARDS), and/or is experiencing respiratory difficulties.

In some embodiments, the treated subject has been diagnosed with or suspected of having a coronavirus infection, optionally COVID-19, and the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing.

In some embodiments, the ophthalmic inflammation may comprise one or more of corneal regeneration, corneal wound healing, corneal melting, dry eye, ocular infection, eyelid sty, and autoimmune-associated peripheral ulcerative keratitis.

In some embodiments, the at least one inflammatory condition or disease may be fibrosis. In exemplary embodiments, the fibrosis may comprise pulmonary fibrosis, cystic fibrosis, idiopathic pulmonary fibrosis, interstitial pulmonary fibrosis, radiation-induced lung injury, liver fibrosis, bridging fibrosis of the liver, cirrhosis, glial scar, arterial stiffness, arthrofibrosis, Crohn's disease, Dupuytren's contracture, keloid fibrosis, Mediastinal fibrosis, Myelofibrosis, Myocardial fibrosis, Peyronie's disease, Nephrogenic systemic fibrosis, Progressive massive fibrosis, pneumoconiosis, Retroperitoneal fibrosis, stromal fibrosis, Scleroderma, systemic sclerosis, chronic obstructive pulmonary disease (COPD), asthma, and adhesive capsulitis.

In some embodiments, the at least one inflammatory condition or disease may be gastrointestinal inflammation. In exemplary embodiments, the gastrointestinal inflammation may comprise inflammatory bowel disease (IBD), Crohn's disease, ulcerative colitis, irritable bowel syndrome (IBS), and Celiac disease.

In some embodiments, the at least one inflammatory condition or disease may be ophthalmic inflammation. In exemplary embodiments, the ophthalmic inflammation may be associated with keratoconjunctivitis sicca.

In some embodiments, the at least one inflammatory condition or disease may be dermatologic inflammation. In exemplary embodiments, the dermatologic inflammation may be selected from eczema and psoriasis.

In some embodiments, the at least one inflammatory condition or disease may be at least one autoimmune disease selected from Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (UP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease.

In exemplary embodiments, a therapeutically or prophylactically effective amount of the cell-free or substantially cell-free regenerative nonsteroidal anti-inflammatory (RNSA) composition may be administered to a subject in need thereof.

In some embodiments, the composition may be administered by one or more of injection, optionally intravenous (IV) or subcutaneous (SC) administration, nebulization, and/or eye drops.

The therapeutically effective amount may comprise one or more doses of the composition. In some embodiments, each dose may range from 0.1 mL/10 kg body weight to 10 mL/10 kg body weight. In some preferred embodiments, the dose may be 1 mL/10 kg body weight. Dosages may be modified or optimized based on criteria selected from a list comprising patient biometrics, specific combination of indications, and preferred route of administration. In some embodiments, each dose may range from 0.01-5 mL for local applications, preferably 1 mL administered SC or directly into locations such as tendons, ligaments, and joints. Each dose may range from 0.01-2 mL for topical eye indications, preferably 0.1 mL administered to each eye as eye drops. Each dose may range from 5-100 mL for systemic use, preferably 8 mL administered by IV infusion. Each dose may range from 0.5-5 mL for nebulization indications, preferably 3 mL administered as a nebulized mist.

In some embodiments, the subject may be selected from a human, non-human primate, pig, sheep, horse, cow, dog, cat, rat, and mouse. In preferred embodiments, the subject may be human.

In some embodiments, the method of treatment or prevention may further comprise the administration of at least one other active, e.g., an anti-inflammatory agent such as an anti-inflammatory antibody or anti-inflammatory fusion protein, an antiviral agent, an antibacterial agent, an antifungal agent, an analgesic, an anti-congestive agent, an anti-fever agent, or a combination of any of the foregoing.

In exemplary embodiments, the subject may have been diagnosed with or is suspected of having a coronavirus infection, optionally COVID-19.

In some embodiments, the subject may have been diagnosed with a coronavirus infection, optionally COVID-19, and is on a respirator, has Acute Respiratory Distress Syndrome, and/or is experiencing respiratory difficulties.

In some embodiments, the subject may have been diagnosed with or suspected of having a coronavirus infection, optionally COVID-19, and optionally the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing.

In some embodiments, the invention provides methods of treatment or prevention of at least one inflammatory or autoimmune condition or disease associated wherein elevated TNFα is associated with the disease pathology or the side effects of a treatment regimen, comprising administering a therapeutically or prophylactically effective amount of a cell-free or substantially cell-free regenerative nonsteroidal anti-inflammatory composition or a cell-free filtrate derived therefrom as herein described, optionally a fraction comprising molecules <1 KDa, to a subject in need thereof.

The inflammatory or autoimmune condition or disease or side effects of a treatment regimen associated with elevated TNFα include any such condition characterized by elevated TNFα. In some embodiments the inflammatory or autoimmune condition or disease or side effects of a treatment regimen comprises one or more of rheumatoid arthritis, psoriatic arthritis, Crohn's disease, fatty liver disease, NASH, asthma, an inflammatory metabolic disorder, optionally type 1 or type 2 diabetes or obesity, inflammatory bowel disease, noninfectious uveitis, sepsis, cytokine storm, cancer, side effects associated with cancer therapy, optionally radiotherapy, chemotherapy, hormone and/or biologic therapy, side effects associated with transplanted cells, tissues and/or or organ, optionally bone marrow transplant, inflammation associated with an acute or chronic viral condition, a neuroinflammatory condition, optionally multiple sclerosis, Alzheimer's disease, migraine, Neuromyelitis optica (NMO), Anti-myelin oligodendrocyte glycoprotein antibody disorder (MOG), Autoimmune encephalitis, Transverse Myelitis, Optic neuritis, neurosarcoidosis, Parkinson's disease, or schizophrenia.

In some of such treatment methods TNFα levels are detected in the treated subject before, during and/or after treatment, optionally by detecting TNFα levels in one or more samples obtained from the subject. Detection of TNFα protein levels or TNFα transcription levels may be detected in biological samples such as blood, serum or plasma by known methods, e.g., using antibody based detection methods such as ELISA, Western blot, immunoblot, radioisotope labeled immune assay, TNFα bioassays (see. or by PCR. In some instances treatment is only effected if TNFα levels are elevated in the subject prior to treatment. In some instances treatment efficacy is monitored after treatment is initiated based on the [reducing] effect of the treatment on TNFα levels, which optionally is determined by detecting TNFα levels in one or more samples obtained from the subject.

In some embodiments, the invention provides methods of activating a Peroxisome proliferator-activated receptor (PPAR), optionally PPARα, PPARβ/δ or (PPARδ), and/or PPARγ in a subject in need thereof by administering a therapeutically or prophylactically effective amount of a cell-free or substantially cell-free regenerative nonsteroidal anti-inflammatory composition or a cell-free filtrate derived therefrom as herein described, optionally a fraction comprising molecules <1 KDa.

In some embodiments the subject has a condition associated with reduced or inhibited activities associated with the activation of Peroxisome proliferator-activated receptors (PPARs), optionally PPARα, PPARβ/δ or (PPARδ), and/or PPARγ such as an autoimmune, inflammatory or cancer condition.

In some embodiments activities elicited by Peroxisome proliferator-activated receptors (PPARs), optionally PPARα, PPARβ/δ or (PPARδ), and/or PPARγ are detected in vivo and/or are detected in one or more samples obtained from the subject before, during and/or after treatment.

In some embodiments Peroxisome proliferator-activated receptor (PPAR) activity, optionally PPARα, PPARβ/δ or (PPARδ), and/or PPARγ activity, is detected in vivo and/or in vitro in one or more samples obtained from the subject after treatment has initiated to assess treatment efficacy.

In some embodiments Peroxisome proliferator-activated receptor (PPAR) activity, optionally PPARα, PPARβ/δ or (PPARδ), and/or PPARγ activity, is detected using one or more methods such as an antibody-based method, optionally immunohistochemistry, immunofluorescence or Western blotting, measurement of PPARγ target gene expression using PCR or a PPAR response element (PPRE) luciferase assay, measurement of PPARγ transcriptional activity, a DNA binding immunoassay that measures the amount of free PPARγ in nuclear extracts or Serum PPARγ Activity Assay (SPAA) (See Edwards et al., Edwards, L., Watt, J., Webster, T. F. et al., “Assessment of total, ligand-induced peroxisome proliferator activated receptor γ ligand activity in serum”, Environ Health 18, 45 (2019). https://doi.org/10.1186/s12940-019-0486-2).

In some embodiments the subject has a metabolic disorder, hypertrophic obesity, insulin-resistance, acute or chronic inflammatory kidney disease, cancer, non-alcoholic fatty liver disease (NAFLD), non-alcoholic steatohepatitis (NASH), fibrosis, Alzheimer's disease (AD), dyslipidemia, hyperglycemia, type 1 or 2 diabetes, a lung disease such as COPD or asthma, a neurodegenerative disorder, obesity, hypertension, atherosclerosis, a cardiovascular condition, vascular injury, heart attack, myocarditis, pericarditis, contractile dysfunction, stroke, hypertension, a neurodegenerative condition, diabetic nephropathy, retinopathy, inflammatory bowel disease, ulcerative colitis, or Crohn's disease (CD).

In some embodiments the invention provides methods of treatment or prevention of at least one condition or disease wherein reactive oxygen species are associated with the disease pathology or wherein reactive oxygen species are side effects associated with a treatment regimen, optionally chemotherapy, radiotherapy, gene therapy, cell therapy, cell, organ and/or tissue transplantation, comprising administering a therapeutically or prophylactically effective amount of a cell-free or substantially cell-free regenerative nonsteroidal anti-inflammatory composition or a cell-free filtrate derived therefrom as herein described, optionally a fraction comprising molecules <1 KDa, to a subject in need thereof.

In some embodiments the condition or disease comprises a respiratory condition such as asthma, chronic obstructive pulmonary diseases, inflammatory bowel disease, neurodegenerative disorders such as Alzheimer's disease (AD), Huntington's disease (HD), amyotrophic lateral sclerosis (ALS), Parkinson's disease (PD), and spinocerebellar ataxia (SCA), cardiovascular diseases such as atherosclerosis, cardiac hypertrophy, ischemic-reperfusion injury, myocyte apoptosis, heart failure, cancers, optionally lung, breast, tongue, gastric, larynx, colon, rectal, lung and prostate cancers. Further optionally cancers wherein ROS contribute to resistance to therapies, metastasis, aberrant angiogenesis and normal or aberrant aging.

In some embodiments the levels of reactive oxygen species (ROS) are detected in the subject or in one or more samples obtained from the subject before, during or after therapy.

In some embodiments the levels of reactive oxygen species (ROS) are detected in the subject or in one or more samples obtained from the subject after therapy has been initiated, optionally to detect treatment efficacy.

In some embodiments the levels of reactive oxygen species (ROS) are detected using one or more techniques, optionally colorimetric assays, immunoblotting, immunofluorescence, flow cytometry, optionally using the FL1 channel (green fluorescence) or FITC channel (see Invitrogen™ Total Reactive Oxygen Species (ROS) Assay Kit 520 nm, Catalog number: 88-5930-74, or a fluorescence plate reader), by fluorescence microscopy, by use of carboxy-H2DCFDA (see Wu D, Yotnda P., “Production and detection of reactive oxygen species (ROS) in cancers”, J Vis Exp. 2011; (57):3357. Published 2011 Nov. 21. doi:10.3791/3357) and by detecting ROS-caused alteration of macromolecules using immunohistochemistry (IHC) (See Liou G Y, Storz P., “Detecting reactive oxygen species by immunohistochemistry”, Methods Mol Biol. 2015; 1292:97-104. doi:10.1007/978-1-4939-2522-3_7).

In some embodiments the invention provides methods of reducing T cell proliferation and/or T cell activation in a subject in need thereof, by administering a therapeutically or prophylactically effective amount of a cell-free or substantially cell-free regenerative nonsteroidal anti-inflammatory composition or a cell-free filtrate derived therefrom as herein described, optionally a fraction comprising molecules <1 KDa.

In some embodiments the subject has an inflammatory or autoimmune condition wherein T cells contribute to the disease pathology.

In some embodiments the condition comprises rheumatoid arthritis (RA), systemic lupus erythematosus (SLE), ITP, CD, IBD, myositis, psoriasis, psoriatic arthritis, vasculitis, scleroderma, type 1 or type 2 diabetes, hypothyroidism, Addison's disease, sepsis, cytokine storm, a neurodegenerative condition, neurological dysfunction or impairment, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, asthma, glomerulonephritis, pancreatitis, hepatitis, inflammatory arthritis, gout, multiple sclerosis, Acute Respiratory Distress Syndrome (ARDS), wound healing, diabetic ulcers, non-healing wounds, lupus, or other autoimmune diseases associated with T cell mediated acute or chronic inflammation.

In some embodiments T cell proliferation and/or T cell activation is detected in the subject or in one or more samples obtained from the subject prior, during or after treatment.

In some embodiments T cell proliferation and/or T cell activation is detected in the subject or in one or more samples obtained from the subject after treatment in order to assess treatment efficacy.

All of the compositions and/or methods disclosed and claimed herein can be made and executed without undue experimentation in light of the present disclosure. While the compositions and methods have been described in terms of preferred embodiments, it will be apparent to those of skill in the art that variations and substitutions may be applied to the compositions and/or methods and in the steps or in the sequence of steps of the methods described herein without departing from the concept, spirit and scope of the disclosure.

### V. Examples

The following examples are provided for illustrative purposes only and are non-limiting.

### Example 1: Generation and Isolation of Regenerative Nonsteroidal Anti-Inflammatory (RNSA) Compositions from Placental Tissue

Human placentas were collected by selective C-section after maternal consent and according to the guidelines of the ethical committee of the Cooperative Human Tissue Network at the University of Alabama. Human placental tissues were processed within 24 hours of collection in a sterile laminar hood as follows.

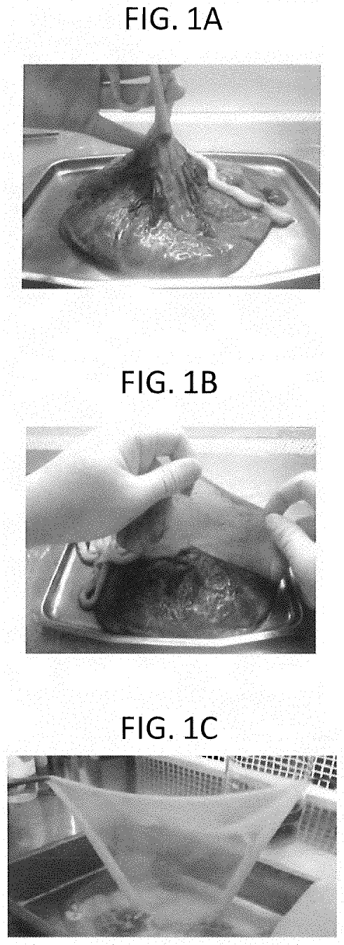

In a laminar flow hood, the amniotic membrane was mechanically separated from the chorion and umbilical cord and subsequently washed extensively with phosphate-buffered saline (PBS) with 1% Primocin™ (Invivogen) (FIG. 1A-C). The amniotic membrane was separated in an Erlenmeyer flask.

The chorion membrane, chorionic villus, and umbilical cord (Wharton's Jelly) were each treated similarly as the amniotic membrane.

The amniotic membrane (or chorion membrane, or chorionic villus, or Wharton's Jelly/umbilical cord tissue) was incubated in a 0.9% NaCl saline solution in an air-tight environment (a capped Erlenmeyer flask) at 37° C. for 10 days with gentle agitation at 90 rpm to induce apoptosis of amniotic membrane cells (i.e., amniotic membrane-derived mesenchymal stromal cells). The supernatant was decanted from the placental tissue and was centrifuged at 10,000×g for 30 min. The supernatant was then filtered through a 0.45 μm membrane and then through a 0.22 μm membrane (VWR) or directly through a 0.22 μm membrane (Pall) to obtain the cell-free or substantially cell-free regenerative nonsteroidal anti-inflammatory composition (“RNSA”). The RNSA composition was stored at −80° C. until use.

### Example 2: Generation and Isolation of Regenerative Nonsteroidal Anti-Inflammatory (RNSA) Compositions from Cultured Placental Mesenchymal Stromal Cells

Human perinatal stromal cells ((hPSCs) which comprise human mesenchymal stromal cells (hMSCs)) were obtained from the placentas described in Example 1 as follows.

The amnion membrane was mechanically separated from the chorion and washed extensively with phosphate-buffered saline (PBS). It was then minced into small pieces and digested with TrypLE (Gibco, Waltham, Mass., USA) at 5 mL/g of tissue for 30 min in a shaker incubator (124 Incubator Shaker series, New Brunswick Scientific, Edison, N.J., USA) at 37° C., 150 rpm to remove the amniotic epithelial cells. The undigested amnion was then removed, washed with PBS and further digested with 125 U/mg Collagenase 1 (Worthington, Lakewood, N.J., USA) at 37° C., 150 rpm for 1.5 h to isolate the amniotic mesenchymal cells (APSC). The mobilized cells in the digest were passed through a 100 μm cell strainer (VWR, Radnor, Pa., USA) and collected by centrifugation at 500×g for 8 min.

Wharton's Jelly (WPSC) was extracted from the umbilical cord as follows: the umbilical cord was sectioned in approximately 1.5 cm in length pieces and then dissected longitudinally to expose the Wharton's Jelly. The arteries and vein were removed, the remaining tissue was minced into small pieces, and digested with 125 U/mg Collagenase 1 at 37° C., 150 rpm for 2.5 h or until all tissue was digested. The digest was passed through a 100 μm cell strainer and centrifuged at 500×g for 8 min.

The chorion was mechanically separated from the amnion membrane and washed extensively with phosphate-buffered saline (PBS). The chorion membrane was then minced into small pieces and digested with 125 U/mg Collagenase 1 (Worthington, Lakewood, N.J., USA) at 37° C., 150 rpm for 1.5 h in a shaker incubator (124 Incubator Shaker series, New Brunswick Scientific, Edison, N.J., USA) to isolate the chorion stromal cells (CSCs). The mobilized cells in the digest were passed through a 100 μm cell strainer (VWR, Radnor, Pa., USA) and collected by centrifugation at 500×g for 8 min. Finally, the placental proper tissue was carefully removed to expose the intermediate and terminal villi, dissected at the base of the intermediate villi and thoroughly washed with PBS, minced into small pieces, and digested with 125 U/mg Collagenase 1 at 37° C., 150 rpm for 1.5 h to isolate the chorionic villi stromal cells (VSCs). The digest was passed through a 100 μm cell strainer and centrifuged at 500×g for 8 min to collect the mobilized perinatal stromal cells (PSCs).

Perinatal Stromal Cells (PSCs) isolated herein had similar characteristics of Mesenchymal Stromal Cells (MSCs) based on the ISCT criteria, which is being plastic adherent under standard culture conditions, expression of CD105+, CD73+, CD90+, CD11b−, and CD45− HLADR. However, the capability of PSCs to differentiate into tri-lineage (Chondrocyte, Osteocyte and Adipocyte) was not evaluated, since it is not suspected that the effect of the compositions derived from PSCs is based on their differentiation capabilities (stemness). Hence, PSCs are referred to herein as MSCs. In addition, some other immune-pertinent markers were tested to further identify such cells. All PSCs were positive (>70%) for CD273+ (PD-L2), CD210+ (IL-10 Receptor) and negative (<5%) for CD178− (FasL), CD119− (IFNg Receptor), CD85d− (ILT4) and CD40. These additional immune-regulatory markers could be used to extend the characterization panel to identify such cells.

The collected hMSCs derived from the amniotic membrane, Wharton's Jelly, chorion membrane and chorionic villus were each cultured using standard procedures known in the art. For each of the above types of placental hMSCs, 10.5 million cultured hMSCs were expanded in a PBS Biotech MINI Bioreactor (RoosterBio) utilizing Synthemax™ II Microcarrier beads (Corning), which the cells adhere to, and 450 mL Rooster Media (Rooster Basal Media supplemented with Rooster Media Booster (RoosterBio)) with agitation at 25 rpm. Daily samples were taken to determine the number of cells per bead by fluorescence microscopy with DAPI staining. On Day 3, 10 mL Rooster Replenish were added to the cells and agitation increased to 30 rpm. Cells were harvested on Day 5 after reaching at least 80% confluence. The culture media was removed and the cells and beads were washed in 300 mL CTS™ Dulbecco's phosphate-buffered saline (PBS) without calcium chloride, without magnesium chloride (ThermoFisher). About 250-300 mL of TrypLE Express (ThermoFisher) were added and incubated for 15 minutes without agitation and for 20 to 40 minutes with agitation at 40 rpm to dispatch cells from the beads. Cells were separated from the beads by filtration and centrifugation.

The cultured and expanded hMSCs were then incubated in 500 mL 0.9% NaCl saline solution in an air-tight environment at 37° C. for about 4 days with gentle agitation (30 rpm) to induce apoptosis. After about 4 days, greater than 95% of the cells will have undergone apoptosis as determined using fluorescence microscopy and staining with the LIVE/DEAD™ Viability/Cytotoxicity Kit (ThermoFisher). The liquid was then decanted and filtered through a 0.22 μm membrane to obtain the cell-free regenerative nonsteroidal anti-inflammatory composition (“RNSA”). The RNSA composition was stored at −80° C. until use.

### Example 3: Characterization of Cell-Free Regenerative Nonsteroidal Anti-Inflammatory (RNSA) Compositions

### Activated Lymphocytes as a Model for Inflammation

Peripheral blood mononucleated cells (PBMCs) were isolated from whole human blood using Lymphoprep™ (STEMCELL™ Technologies) according to the manufacturer's protocol. T cells were isolated from the PBMCs using EasySep™ Release Human CD3 Positive Selection Kit (STEMCELL™ Technologies) according to the manufacturer's protocol. The T cells were then activated and expanded using ImmnoCult™ CD3/CD28/CD2 T cell activator (STEMCELL™ Technologies) according to the manufacturer's protocol. The activated T cell samples are referred to herein as “Tc3+Act” whereas the T cell samples which were not activated are referred to herein as “Tc3-Act.”

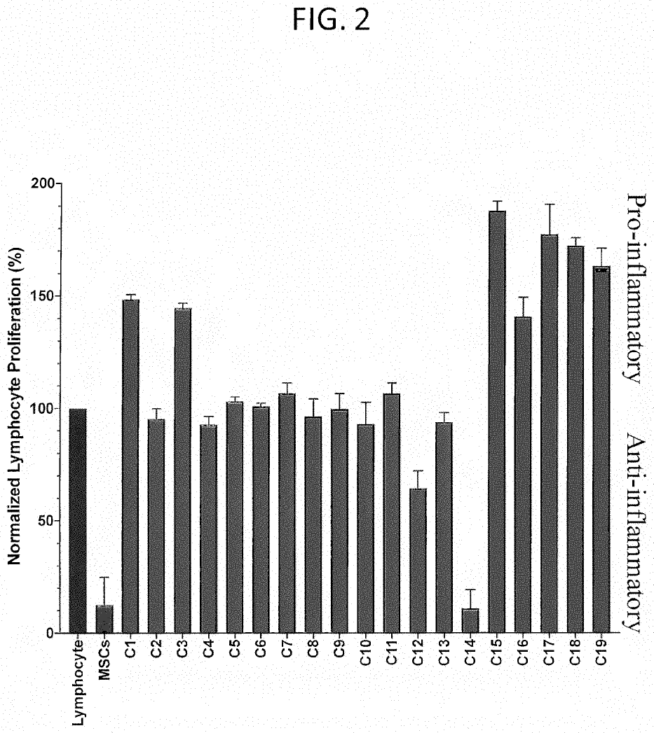

The activated and expanded T lymphocytes described above are a model for inflammation in which the normalized T cell proliferation represents 100% (FIG. 2). When the activated T cells are cultured in the presence of MSCs, inhibition of T cell proliferation of greater than 85% is observed (FIG. 2), representing a positive control for the inhibition of inflammation. Numerous tissues and conditions for treating those tissues were explored to determine a cell-free regenerative anti-inflammatory composition, including the amniotic membrane tissue and stress-induced lysis condition described in Example 1 to produce the cell-free RNSA, shown in FIG. 2 as “C14.” The results for eighteen other conditions in which a cell-free composition was prepared and tested for its ability to inhibit activated T cell proliferation are also shown in FIG. 2. Of these nineteen conditions, only the cell-free regenerative anti-inflammatory composition derived from amniotic membrane tissue described in Example 1 inhibited activated T cell proliferation with a potency equal to or greater than MSCs (FIG. 2). Most conditions tested had no effect, whereas several conditions tested produced pro-inflammatory extracts (FIG. 2).

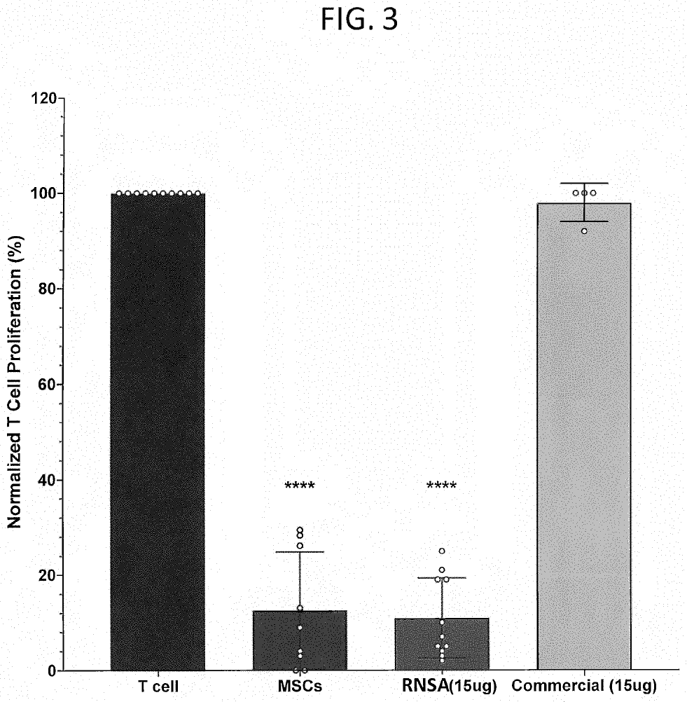

As described above, the activated and expanded T lymphocytes are a model for inflammation in which the normalized T cell proliferation represents 100% (FIG. 3). When the activated T cells are cultured in the presence of MSCs, inhibition of T cell proliferation of greater than 85% is observed (FIG. 3), representing a positive control for the inhibition of inflammation. Alternatively, when the activated T cells are cultured in the presence of 15 μg of the cell-free regenerative nonsteroidal anti-inflammatory composition (“RNSA”) derived from aminiotic membrane tissue described in Example 1, inhibition of T cell proliferation of greater than 85% is also observed (FIG. 3). In contrast, when the activated T cells are cultured in the presence of 15 μg of a commercial cell-free reagent (“commercial”), no inhibition of T cell proliferation was observed (FIG. 3).

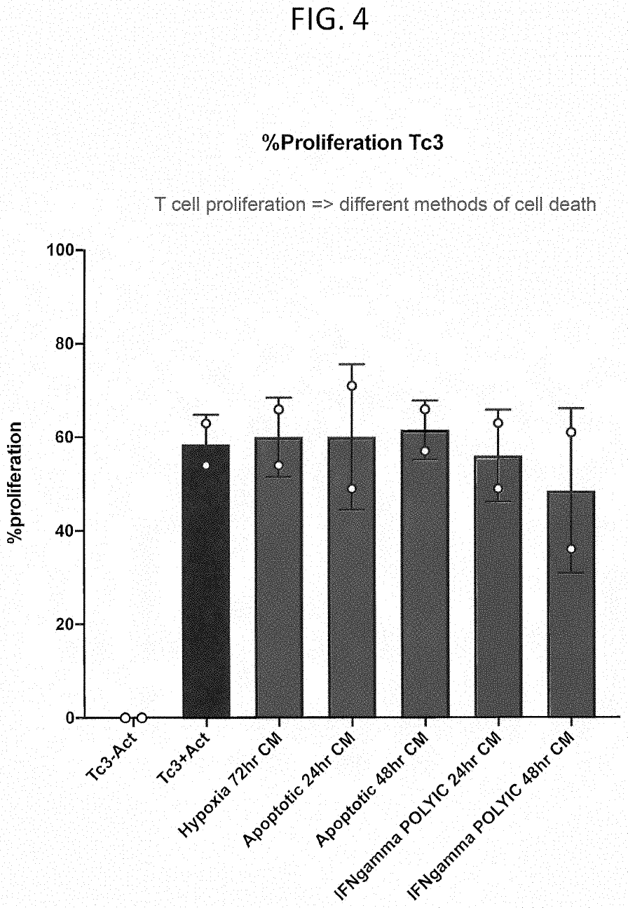

To determine whether the method of cell death has an effect on the potency of the RNSA composition, the protocol for generating the RNSA from amniotic membrane described in Example 1 was altered such that various means of cell death were tested. The amniotic tissue was subjected to a hypoxia condition for 72 hours, a stress-induced lysis condition for 24 hours or 48 hours, or to IFN gamma and Poly(I:C) dsRNA for 24 hours or 48 hours. As shown in FIG. 4, none of these conditions produced an RNSA composition which inhibited activated T cell proliferation. This is in contrast to the amniotic membrane RNSA composition described in Example 1, which was produced using a stress-induced lysis condition for 10 hours (FIG. 2 and FIG. 3).

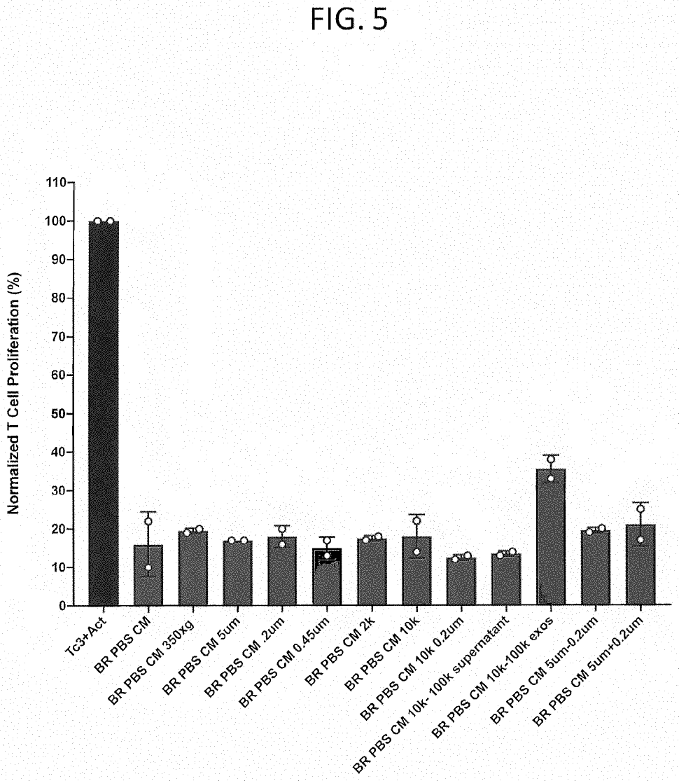

The T cell proliferation assay was also used to determine the effect of filtration on the potency of the RNSA composition. The RNSA composition was centrifuged at 350×g or filtered through membranes of various size cutoffs, including 5 μm, 0.45 μm, 0.2 μm, 2 KDa MWCO and 10 KDa MWCO, as shown in FIG. 5. Also tested were a 10 KDa-100 KDa filtration supernatant, and isolated exosomes “exon” from the supernatant, also shown in FIG. 5. The RNSA composition produced from amniotic membrane-derived hMSCs expanded in the PBS bioreactor (“BR PBS”) described in Example 2 was used in these tests. Condition media (“CM”) indicates the apoptotic condition of Examples 1 and 2. These results indicate that the bioactive molecule(s) of the RNSA composition is/are small molecule(s) less than 2 KDa, and that exosomes alone inhibited activated T cell proliferation less than the non-exosome fractions.

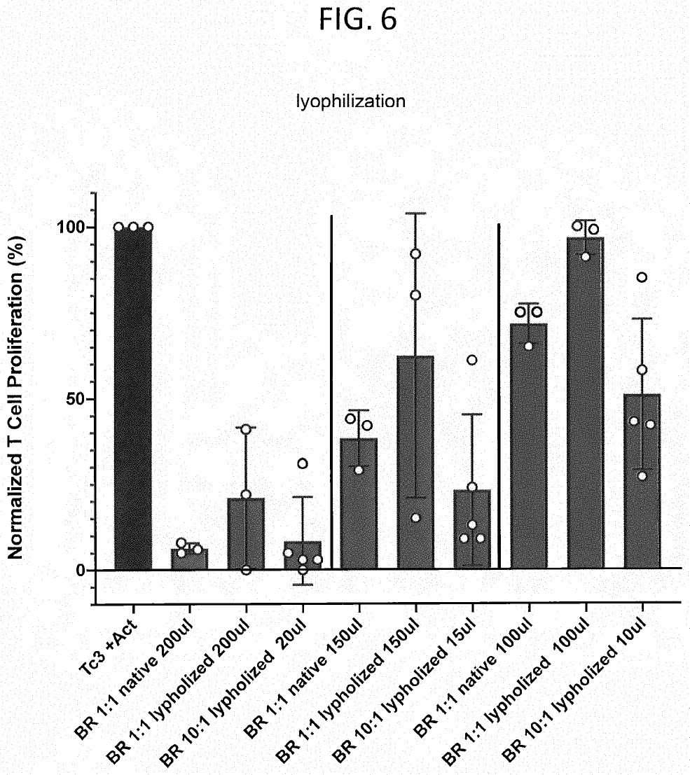

The T cell proliferation assay was also used to determine the effect of lyophilization on the potency of the RNSA composition. Here, the RNSA composition produced using cultured amnion hMSCs from Example 2 was used. Native RNSA samples of 200 μL, 150 μL, and 100 μL were compared to the same volume of native RNSA samples which were lyophilized and then resuspended in the equivalent volume of deionized water or 10-fold lower volume of deionized water. As shown in FIG. 6, lyophilization did not inhibit the bioactivity of the RNSA composition.

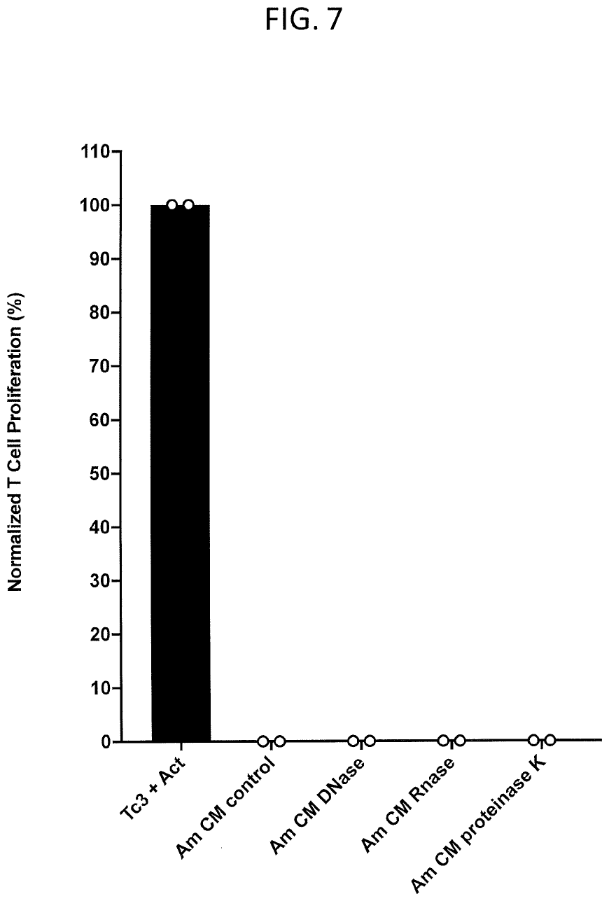

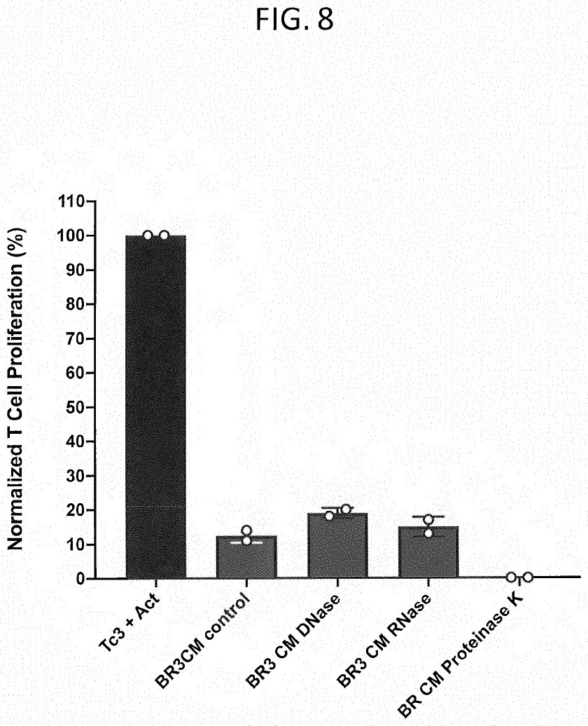

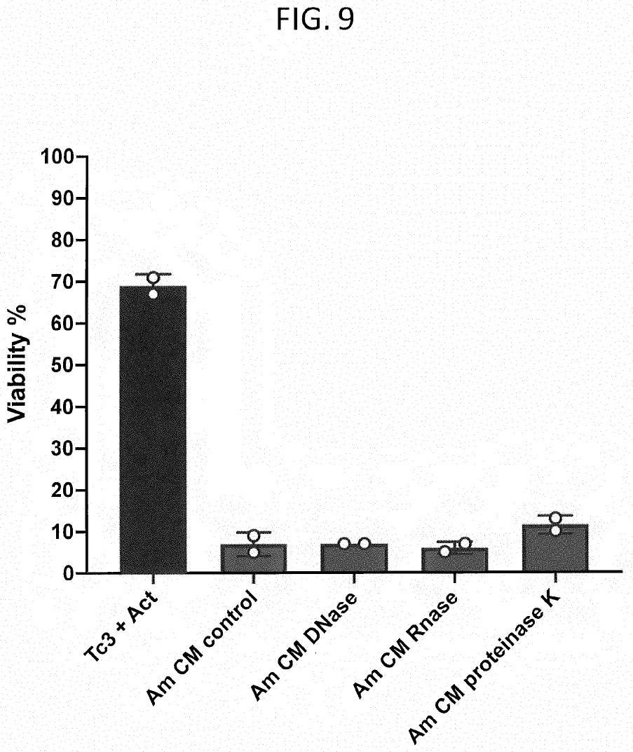

The T cell proliferation assay was also used to determine the effect of proteases, DNase, and RNase on the potency of the RNSA composition. As shown in FIG. 7, the potency of the RNSA composition produced from amniotic membrane tissue extraction described in Example 1 was not affected by DNase, RNase, or Proteinase K, suggesting a lipid nature for the bioactive compound(s). Similarly, as shown in FIG. 8, the potency of the RNSA composition produced from cultured hMSCs described in Example 2 was not affected by DNase, RNase, or Proteinase K. And as shown in FIG. 9, a similar T cell viability assay was used to determine that Proteinase K, DNase, and RNase do not affect the potency of the RNSA composition produced from amniotic membrane tissue extraction described in Example 1.

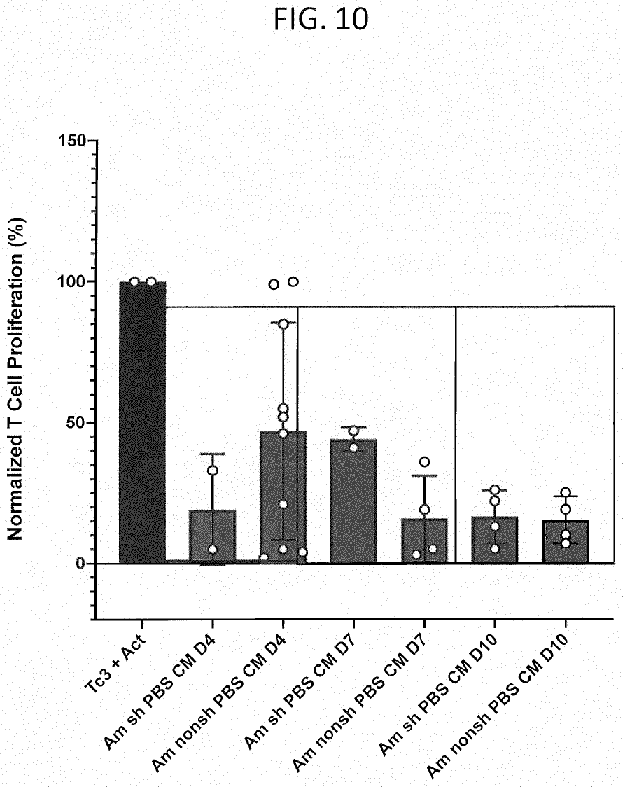

The T cell proliferation assay was also used to determine the effect of time during extraction and shaking vs. non-shaking conditions on the potency of the RNSA composition produced from amniotic membrane tissue extraction described in Example 1. As shown in FIG. 10, better extraction uniformity was achieved when waiting until Day 10. Also shown in FIG. 10, there was not a big effect from shaking vs. non-shaking when waiting until Day 10.

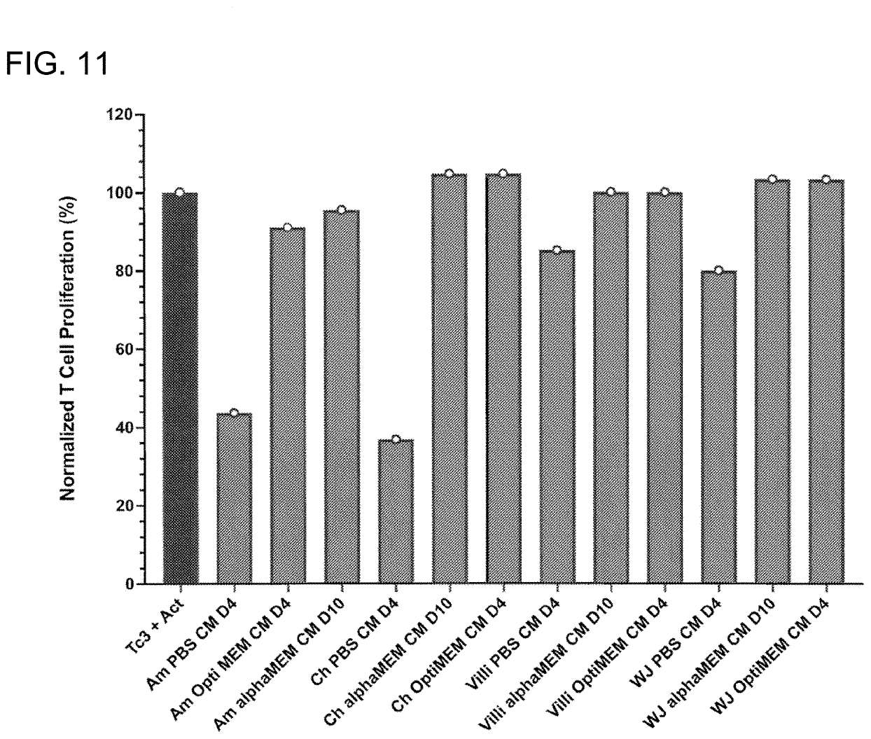

The T cell proliferation assay was also used to determine the effect of tissue type (AM—amniotic membrane, CH—chorion membrane, Villi—chorionic villus, WJ—Wharton's Jelly/Umbilical cord tissue) on the potency of the RNSA composition produced from the placental tissue extractions described in Example 1. The effect of the culture media (AlphaMEM, OptiMEM, and PBS) was also tested. As shown in FIG. 11, the RNSA compositions produced from amniotic membrane tissue extraction in PBS and from chorion membrane tissue extraction in PBS had the highest potency.

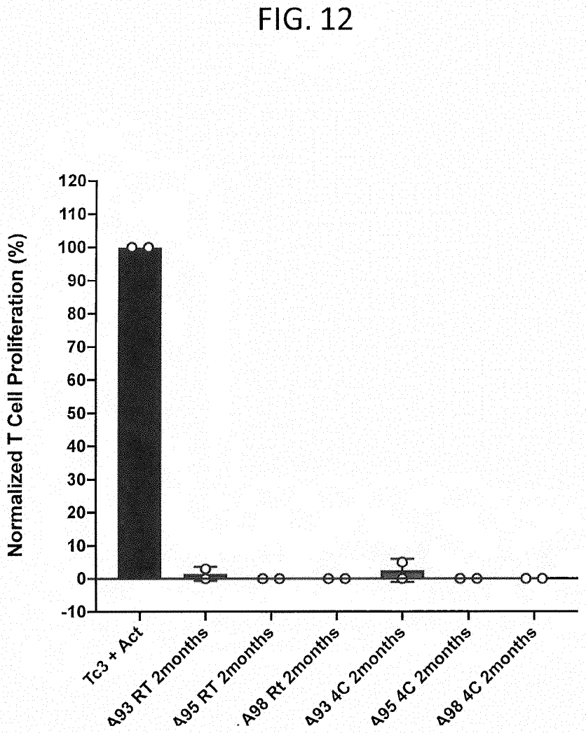

The T cell proliferation assay was also used to determine the stability of the RNSA composition at room temperature and 4° C. As shown in FIG. 12, various RNSA compositions (designated A93, A95, and A98) maintained potency for 2 months (greater than 8 weeks) at both room temperature and 4° C.

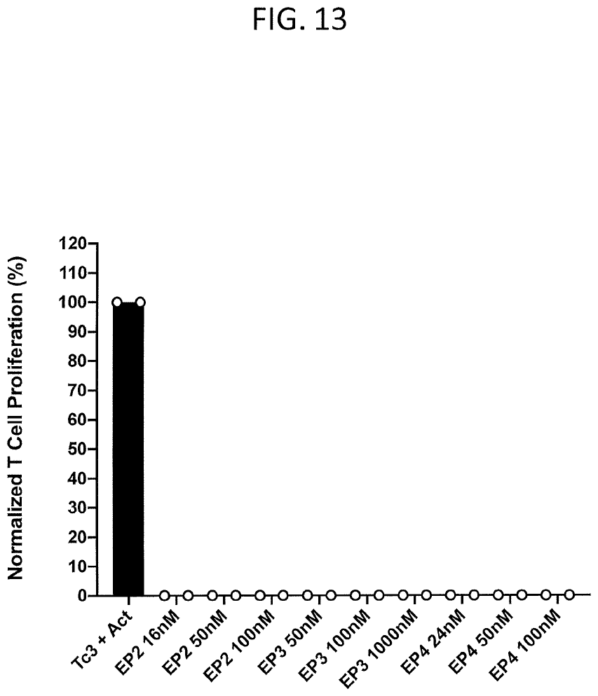

The T cell proliferation assay was also used to determine the effect of various concentrations of the EP2, EP3, and EP4 receptor blockers on the potency of the RNSA composition. As shown in FIG. 13, the EP receptor blockers did not affect the potency of the RNSA composition.

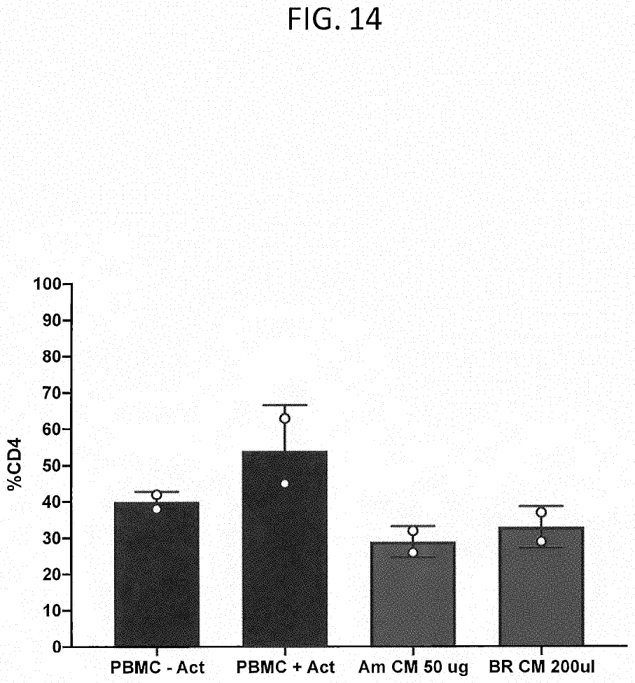

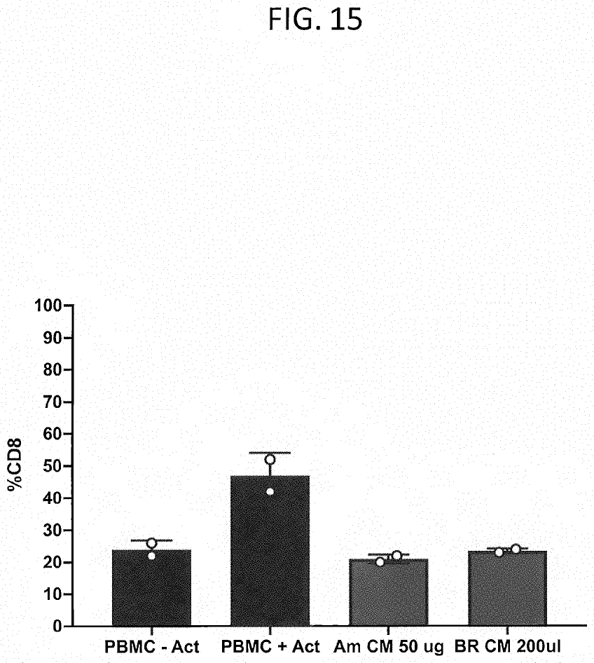

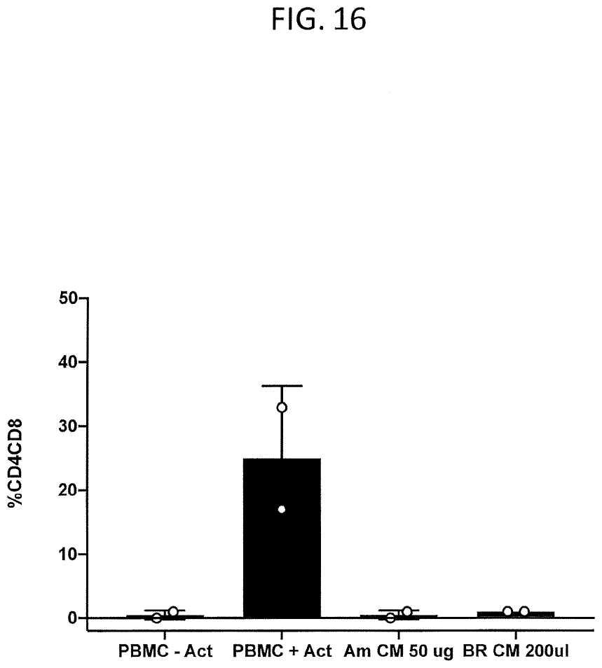

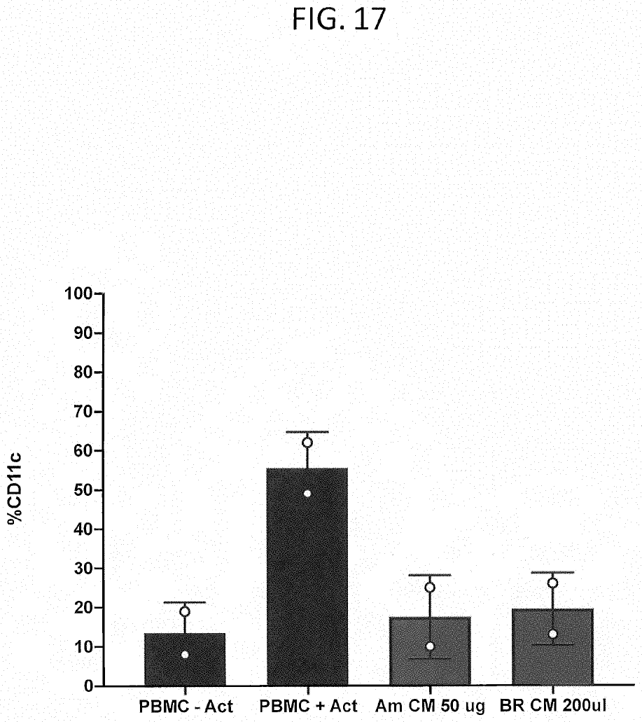

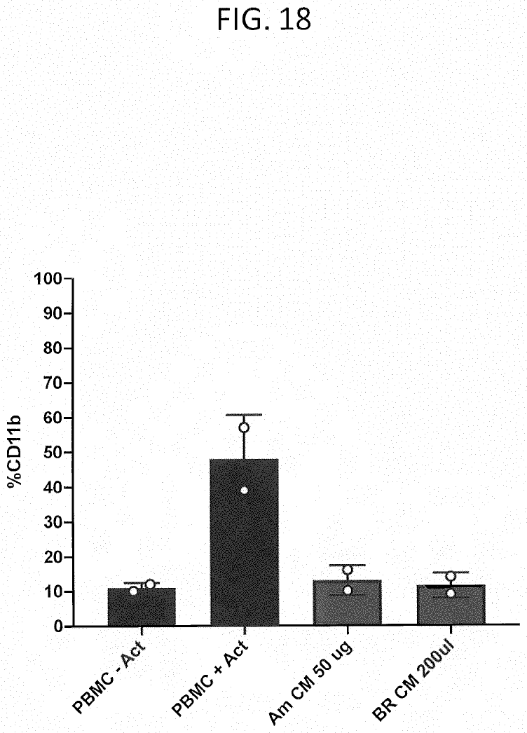

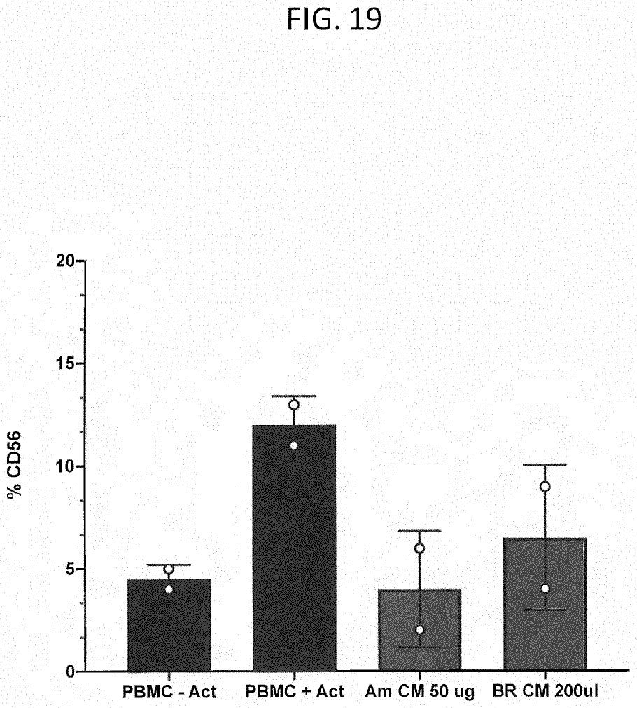

In some instances, a modified version of the T cell proliferation assay was utilized in which T cells were not isolated from the PBMCs. Rather, PBMCs were activated with the CD3/CD28/CD2 T cell activator and proliferation of CD4+ T cells was monitored. This modified proliferation assay was used to determine the effect of the tissue extraction process described in Example 1 (“AM”) compared to the cultured MSCs extraction process described in Example 2 (“BR”), as shown in FIG. 14, on the potency of the RNSA compositions. FIG. 15 shows the same modified T cell proliferation assay except that proliferation of CD8+ T cells was monitored. FIG. 16 shows the same modified T cell proliferation assay except that proliferation of CD4+/CD8+ T cells was monitored. FIG. 17 shows the same modified T cell proliferation assay except that proliferation of CD11c+ T cells was monitored. FIG. 18 shows the same modified T cell proliferation assay except that proliferation of CD11b+ T cells was monitored. FIG. 19 shows the same modified T cell proliferation assay except that proliferation of CD56+ T cells was monitored.

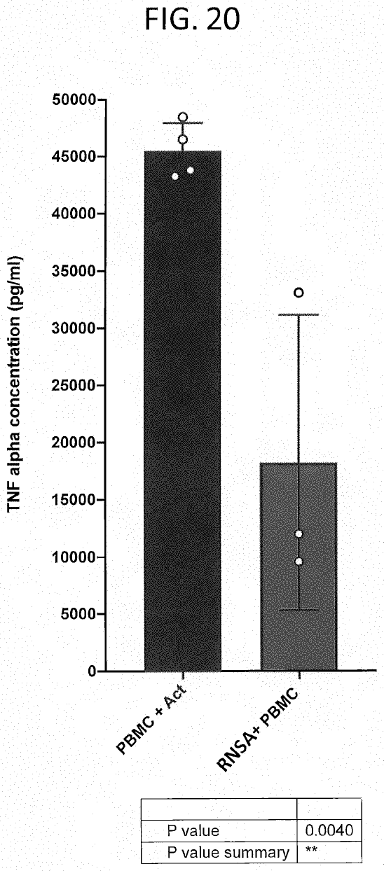

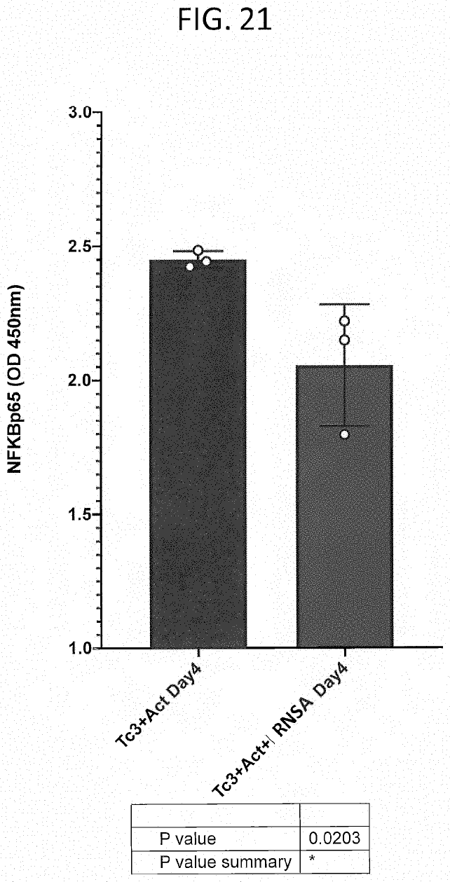

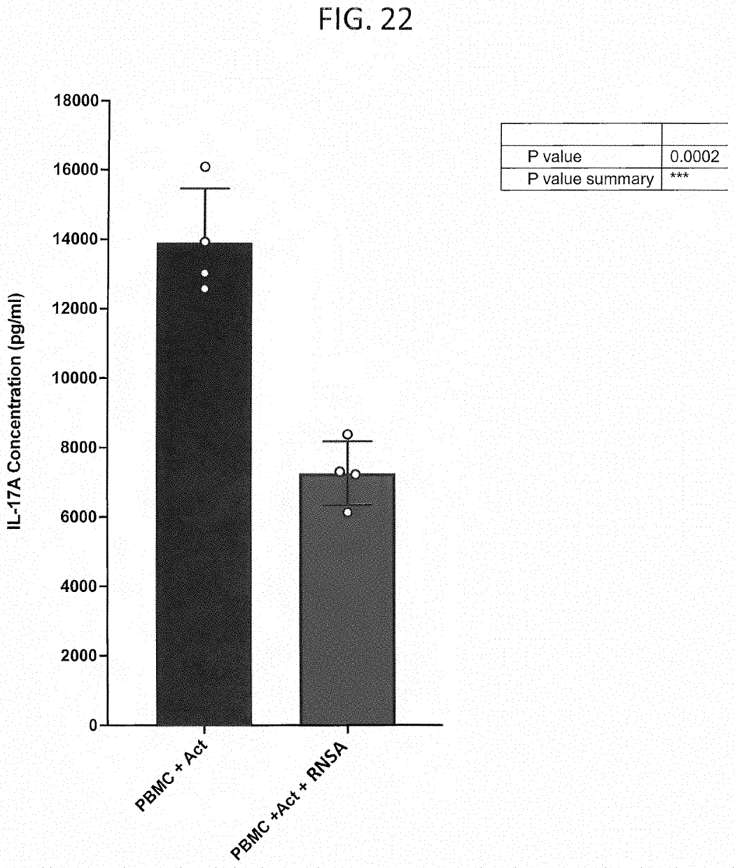

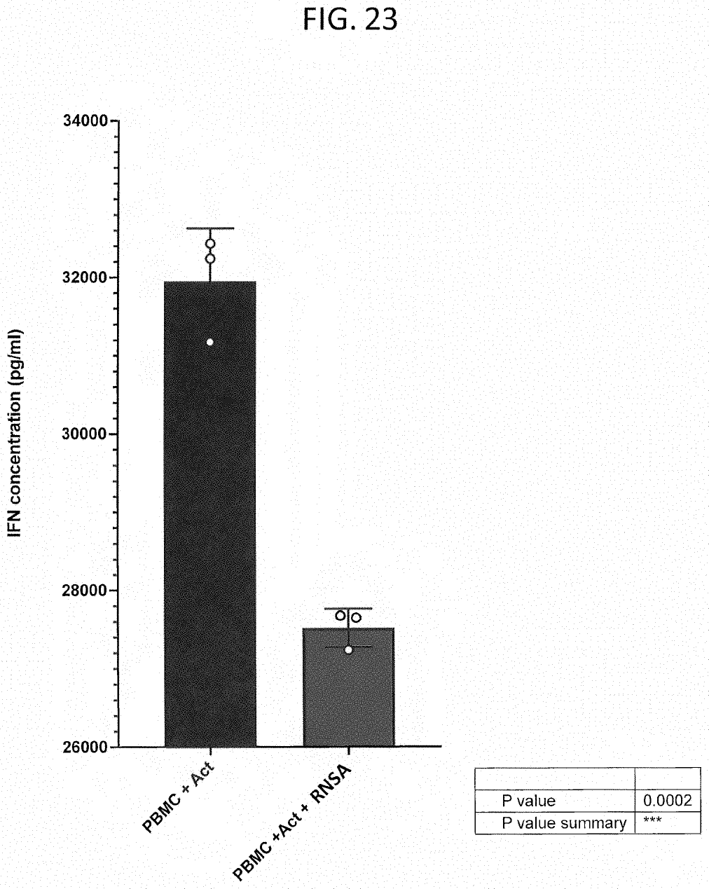

Next, the effect of the RNSA composition on the expression of pro-inflammatory cytokines was determined. Briefly, the amounts of pro-inflammatory cytokines were quantified in activated PBMCs in the absence and presence of the RNSA composition. FIG. 20 shows that the RNSA composition reduces the expression level of TNFα from activated PBMCs. FIG. 21 shows that the RNSA composition reduces the expression level of NFκB from activated PBMCs. FIG. 22 shows that the RNSA composition reduces the expression level of IL-17A from activated PBMCs. FIG. 23 shows that the RNSA composition reduces the expression level of IFNγ from activated PBMCs.

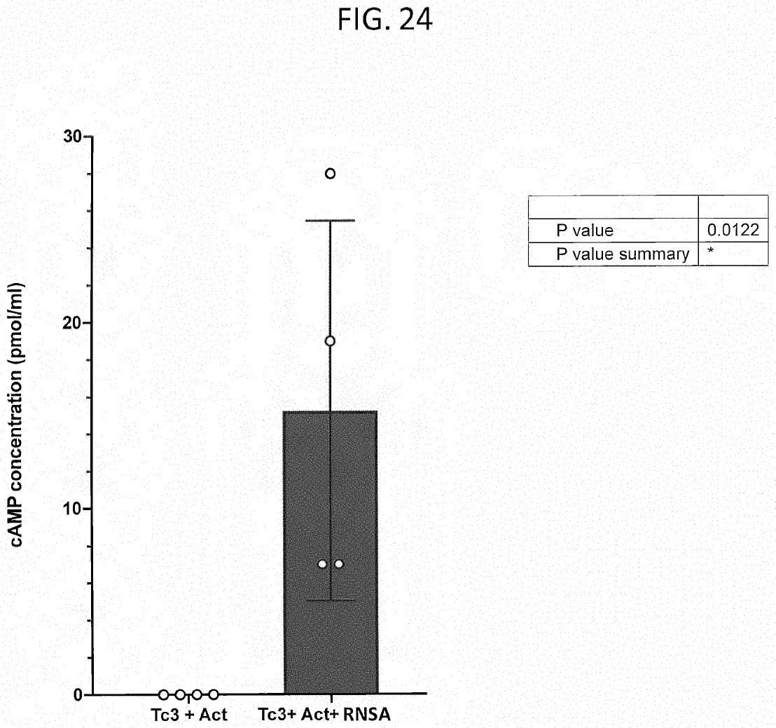

Next, the effect of the RNSA composition on the production of cAMP by activated T cells was determined. FIG. 24 shows that the RNSA composition promotes/induces cAMP production by activated T cells.

### Characterization of Eicosanoid Compounds in RNSA Compositions

Various samples were analyzed by mass spectrometry to determine the identity and concentrations of eicosanoid compounds in the RNSA composition. The analyzed samples included three samples extracted in αMEM media (αMEM-1, αMEM-2, and αMEM-3) which served as negative controls. Four RNSA composition samples produced using the cultured hMSCs described in Example 2 (BR-1, BR-2, BR-3, and BR-4) and three RNSA composition samples produced using the amniotic membrane extraction protocol described in Example 1 (CM-1, CM-2, and CM-3) were also analyzed.

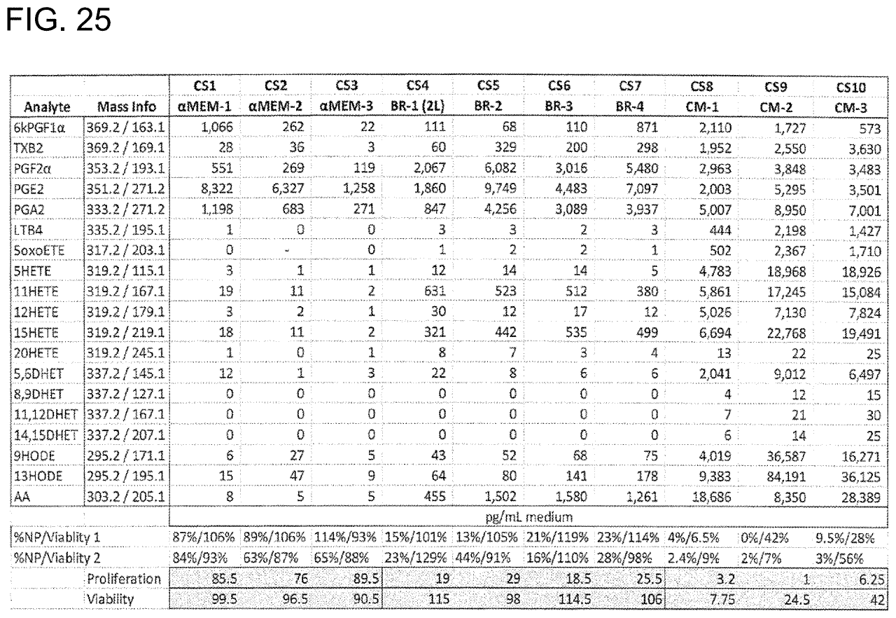

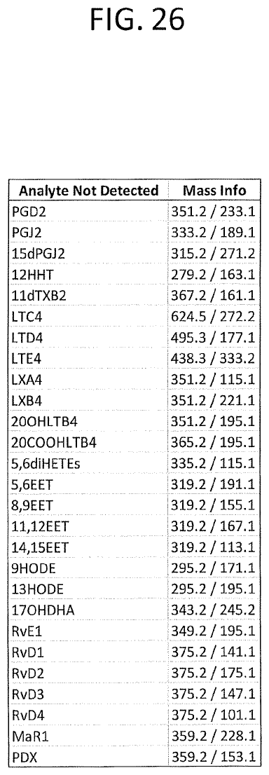

Results of the eicosanoid analysis are shown in FIG. 25. The concentration of each eicosanoid shown in the Analyte column was determined and is shown in pg/mL media. The analyzed compositions were also tested in the T cell proliferation and viability assays described above, the results of which are shown in the bottom two rows, respectively, of FIG. 25. It is clear from this analysis that the CM-1, CM-2, and CM-3 RNSA compositions are enriched for several eicosanoids compared to the αMEM and BR samples. Further, the CM samples inhibited T cell proliferation and reduced T cell viability, whereas the BR samples inhibited T cell proliferation to a lesser extent and did not reduce T cell viability. Several eicosanoid analytes were not detected in any of the analyzed samples, which compounds are shown in FIG. 26.

### Cell Proliferation as a Model for Regeneration

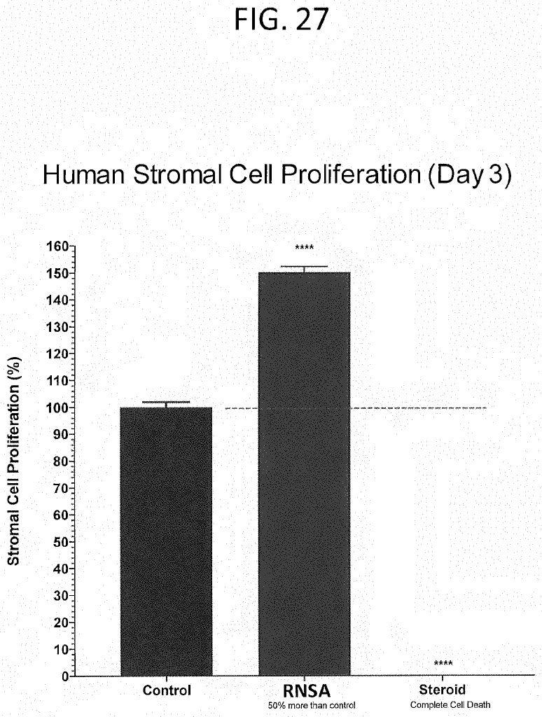

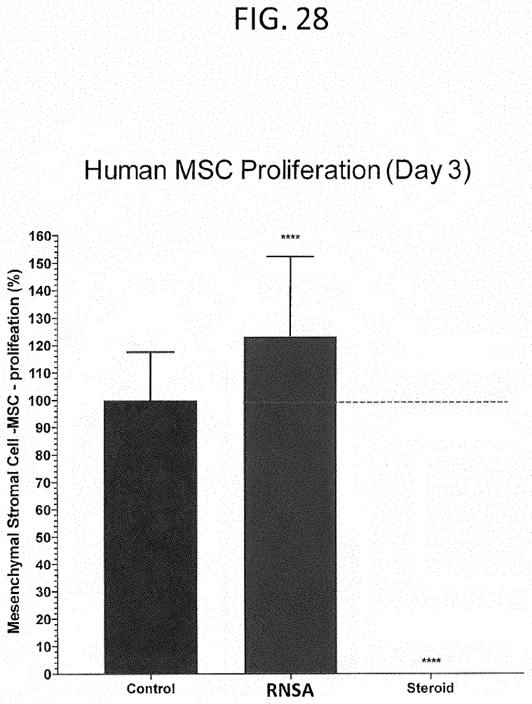

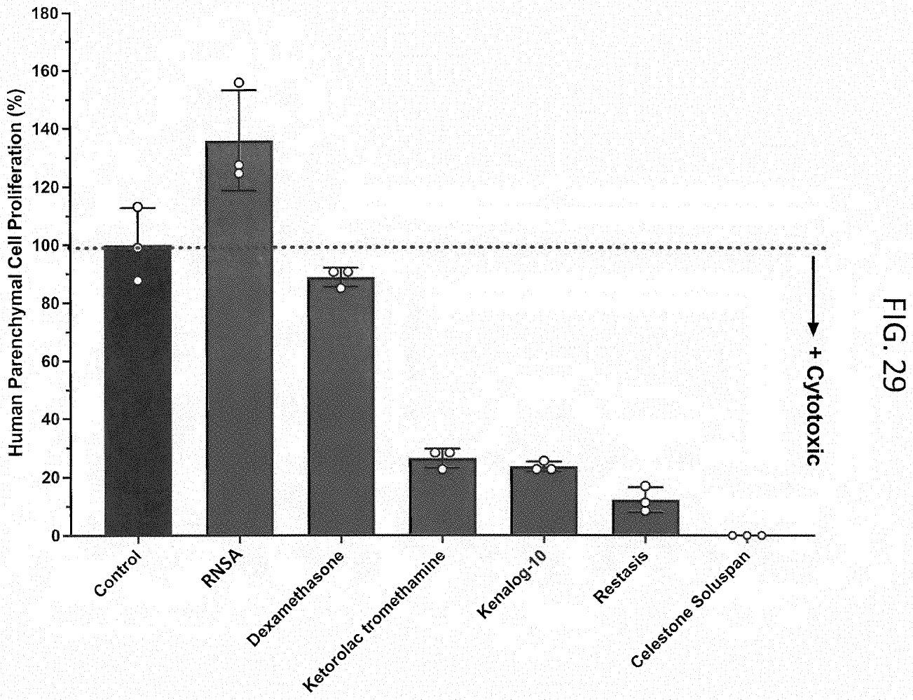

In order to test the regenerative properties of the RNSA composition, human stromal cells and human mesenchymal stromal cells (hMSC) were cultured in the absence or presence of the RNSA composition or in the absence or presence of a steroid. As shown in FIG. 27 and FIG. 28, the RNSA composition promotes proliferation of human stromal cells and hMSCs, respectively, whereas the steroid is completely cytotoxic.

Similarly, the regenerative properties of the RNSA composition were tested using human parenchymal cells. As shown in FIG. 29, the RNSA composition promotes proliferation whereas the tested commercial compounds were each cytotoxic to varying degrees.

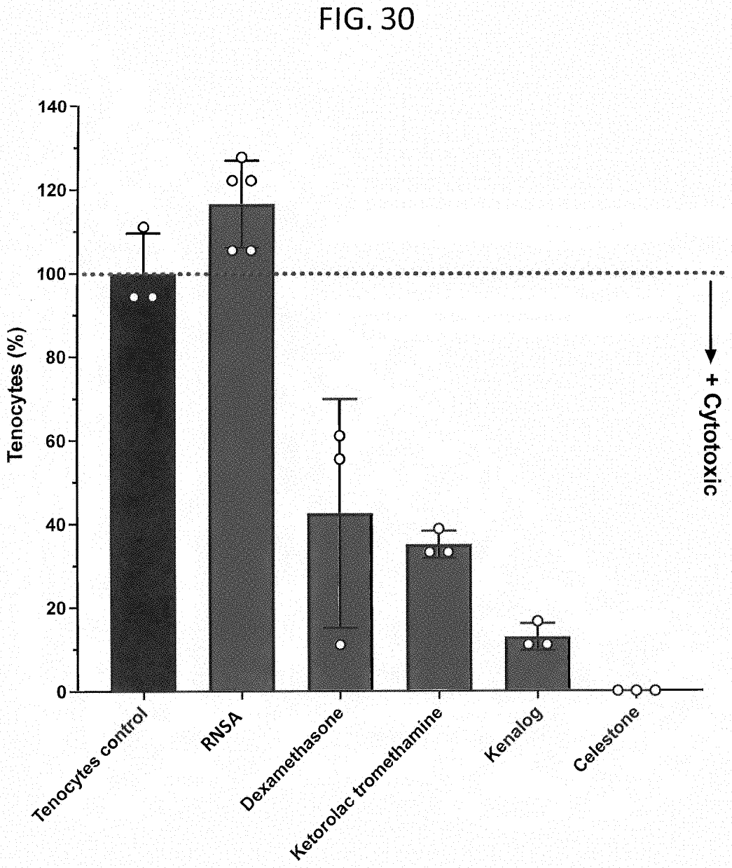

Similarly, the regenerative properties of the RNSA composition were tested using human tenocytes. As shown in FIG. 30, the RNSA composition promotes proliferation whereas the tested commercial compounds were each cytotoxic to varying degrees.

### Example 4: Evaluation of the Effect of Cell-Free Regenerative Nonsteroidal Anti-Inflammatory (RNSA) Compositions on a Model of Humanized Graft Versus Host Disease

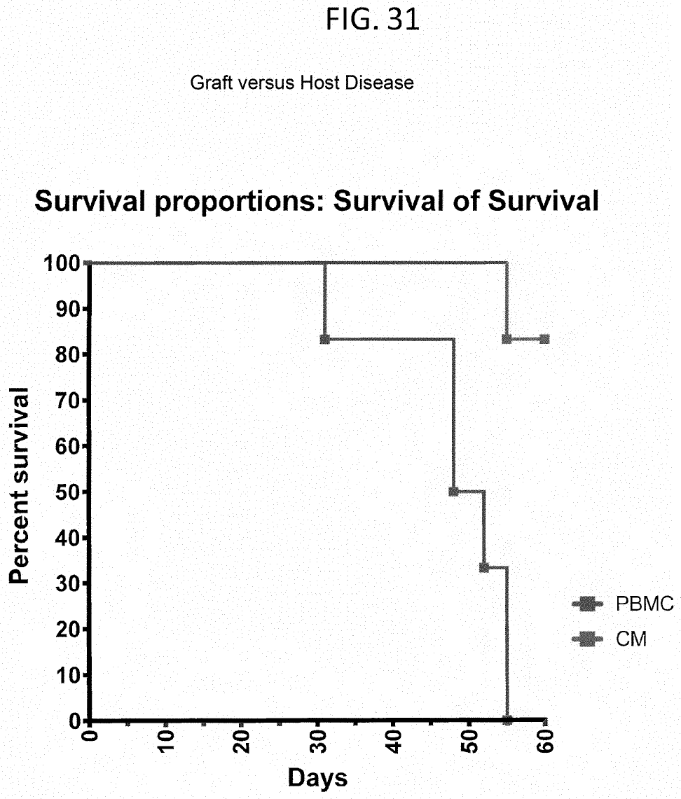

A humanized mouse model of Graft versus Host Disease (GvHD) was used to evaluate the effect of the RNSA composition. Briefly, the mice lacking an immune system are injected with human PBMCs, which begin attacking the murine tissues. As shown in the survival curve in FIG. 31, by day 50, half of the control mice injected only with media (PBMC) have died. In contrast, administration of the cell-free regenerative nonsteroidal anti-inflammatory composition (CM) rescues the GvHD mice such that by day 50, 100% of the treated mice are still alive.

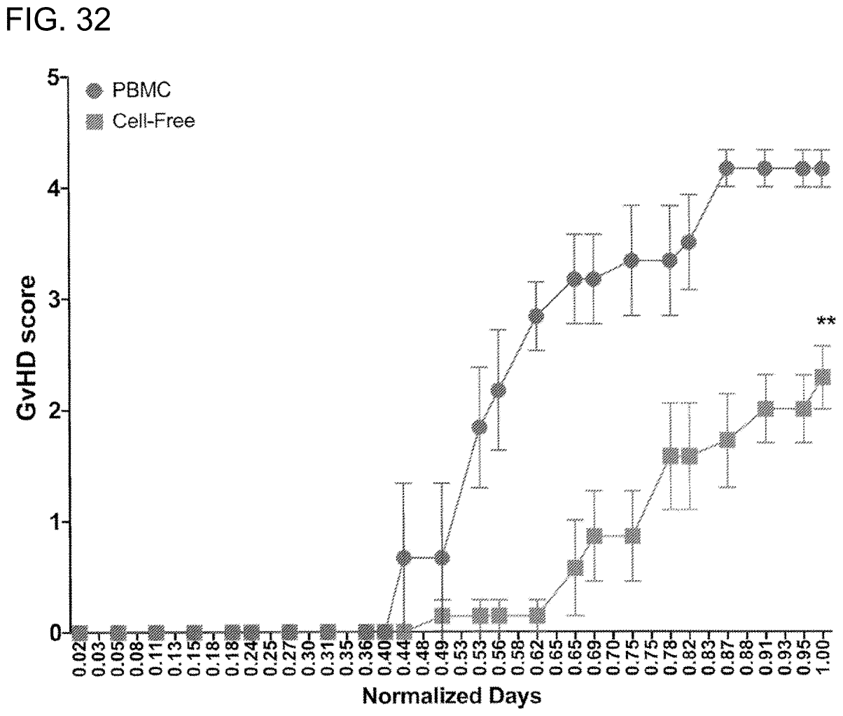

FIG. 32 is a plot of the GvHD score versus normalized days for the GvHD mice injected with media (PBMC) or with an exemplary cell-free regenerative nonsteroidal anti-inflammatory composition (Cell-Free) according to the invention. A higher GvHD score represents a poorer prognosis.

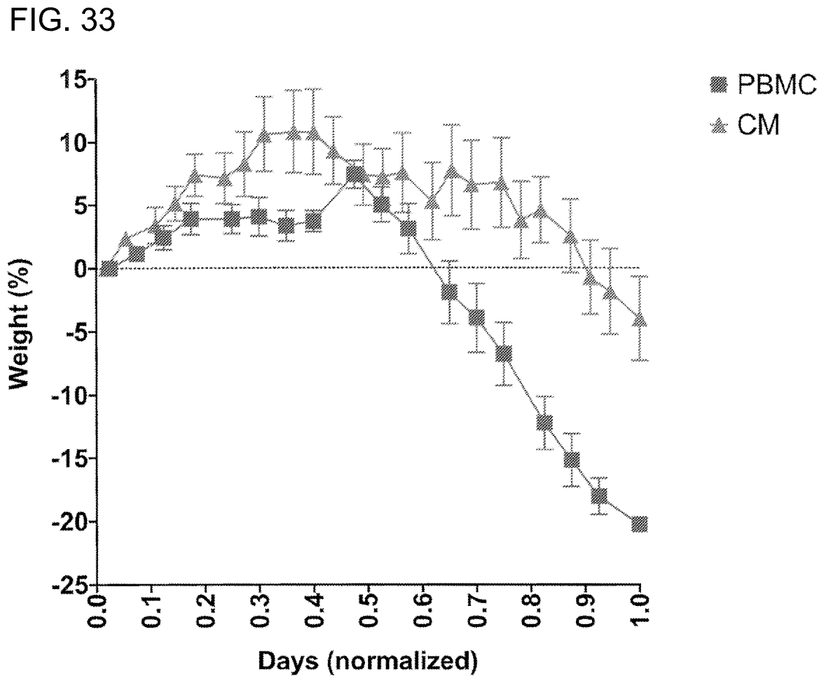

FIG. 33 is a plot of the body weight GvHD mice injected with media (PBMC) or with an exemplary cell-free regenerative nonsteroidal anti-inflammatory composition (CM) according to the invention. A drop in weight reflects the severity of the autoimmunity progression of the disease. The RNSA composition has a strong effect in ameliorating the severity of GvHD.

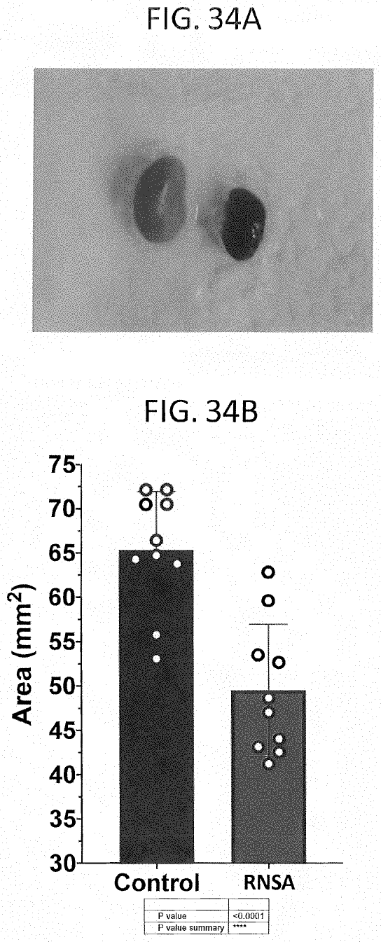

FIG. 34A is a photo of GvHD mouse model kidneys. The kidney on the left from the control mouse is enlarged indicating it is inflamed and likely fibrotic, whereas the kidney on the right is from the GvHD mouse treated with the RNSA composition. The kidney on the right looks normal. FIG. 34B is a plot of the GvHD mouse kidney areas in mm².

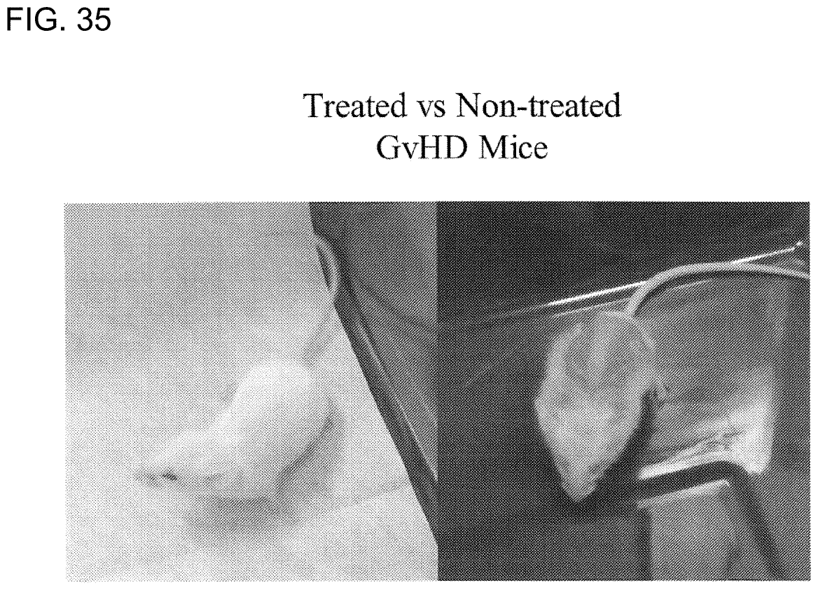

FIG. 35 is a photo of the GvHD mice. A GvHD mouse treated with an exemplary RNSA composition according to the invention is shown on the left whereas an untreated GvHD mouse is shown on the right.

### Example 5: Evaluation of the Effect of Cell-Free Regenerative Nonsteroidal Anti-Inflammatory (RNSA) Compositions on Cardiomyopathy

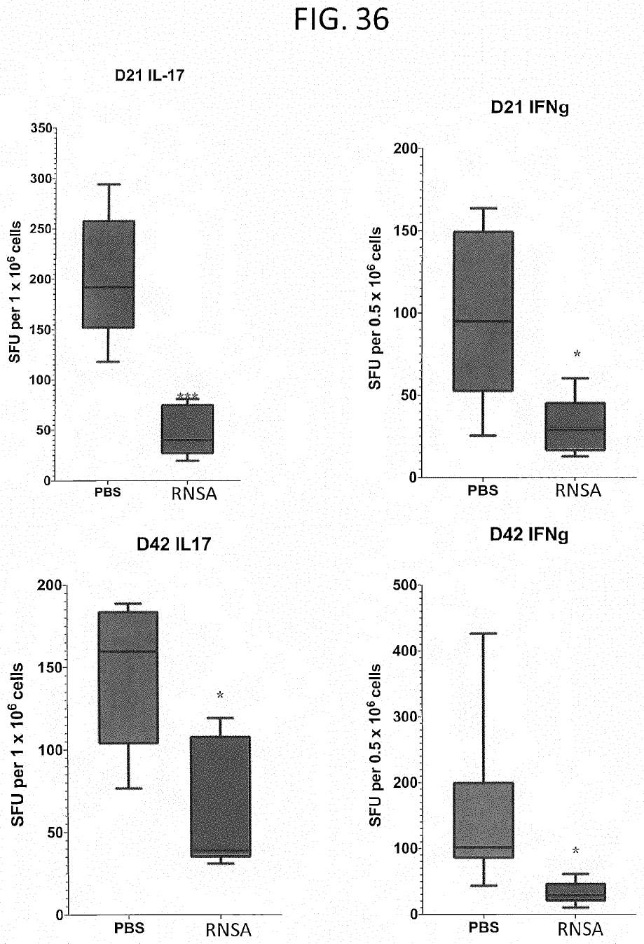

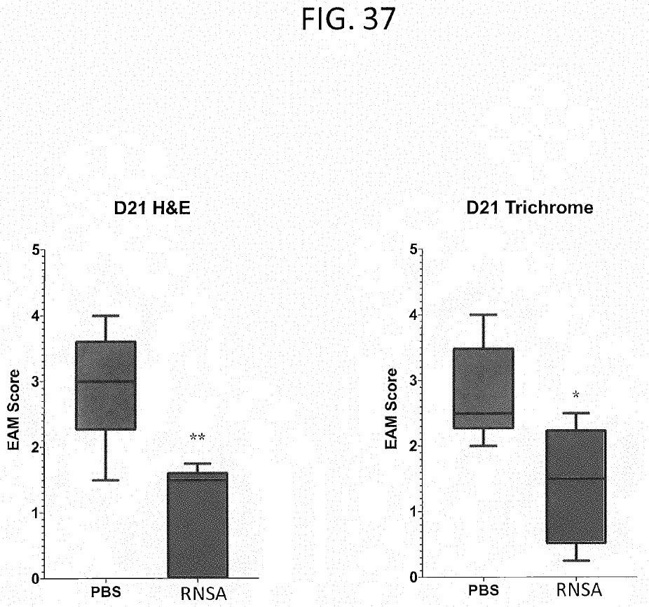

In a mouse model of cardiomyopathy (EAM), the RNSA composition reduced inflammatory markers IL-17 and IFNγ in vivo at Day 21 and Day 42 as determined using an ELISpot assay compared to the untreated control mouse (PBS) (FIG. 36). The RNSA composition also reduced inflammatory markers histopathology disease scores and Trichrome fibrosis scores at Day 21 compared to the untreated control mouse (FIG. 37).

### Example 6: Evaluation of the Effect of Cell-Free Regenerative Nonsteroidal Anti-Inflammatory (RNSA) Compositions on Eczema

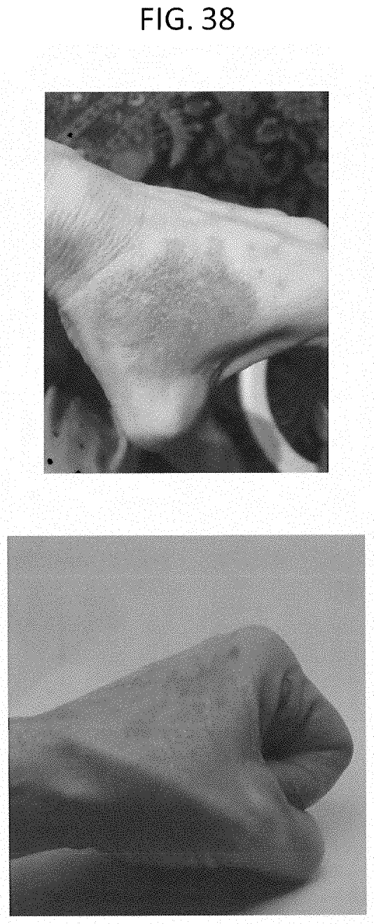

A human patient having eczema which was non-responsive to traditional treatment was treated with a first subcutaneous injection dose of an exemplary RNSA composition at the site of eczema (patient's left hand) on Day 1 and with a second subcutaneous injection dose on Day 30. FIG. 38 shows the patient's left hand on Day 1 and on Day 60. After 60 days, the patient's eczema was completely cleared.

### Example 7: Evaluation of the Effect of Cell-Free Regenerative Nonsteroidal Anti-Inflammatory (RNSA) Compositions on Tendinopathies

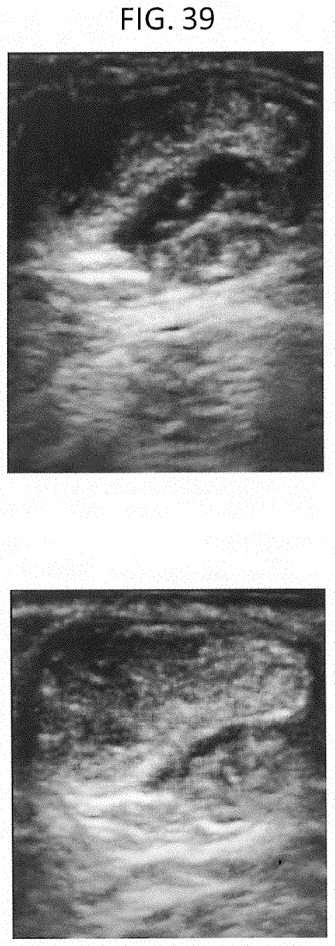

A human patient having tendinosis of the ankle and non-responsive to traditional treatments was treated with a single subcutaneous injection dose of an exemplary RNSA composition according to the invention at the site of tendinosis. FIG. 39 shows an ultrasound image of the tendinosis before treatment and an image of the healed tendon 30 days after treatment.

### Example 8: Evaluation of the Effect of Cell-Free Regenerative Nonsteroidal Anti-Inflammatory (RNSA) Compositions on COVID-19-Associated Pneumonia

Human patients having COVID-19-Associated Pneumonia are treated with the RNSA composition. Specifically, patients receive one dose of 1 mL of an exemplary RNSA composition according to the invention for each 10 kg of body weight into the blood stream via I.V. infusion. Patients are monitored by the attending physician(s) and hospital staff during the course of their hospital stay. Patients are evaluated by x-ray of the lungs at one month after the patient is discharged from the hospital. Patients receiving RNSA composition show a statistically significant improvement (i.e. decrease) of one or more symptoms of COVID-19-Associated pneumonia compared to patients receiving placebo.

### Example 9: Evaluation of the Effect of Cell-Free Regenerative Nonsteroidal Anti-Inflammatory (RNSA) Composition on Eyelid Sty

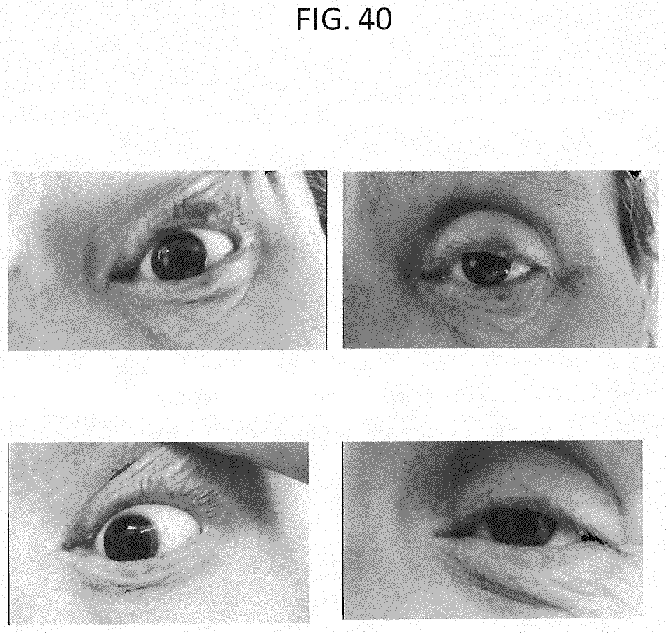

A human patient having an eyelid sty was treated with eye drops comprising an exemplary RNSA composition according to the invention. FIG. 40 shows two photos of the patient's eye having the eyelid sty prior to the treatment (top) and two phots of the same eye six weeks after treatment showing the eyelid had fully healed (bottom). The patient did not receive any other treatment or procedure for the eyelid stye.

### Example 10: Quantitation of Free Fatty Acids in RNSA Compositions

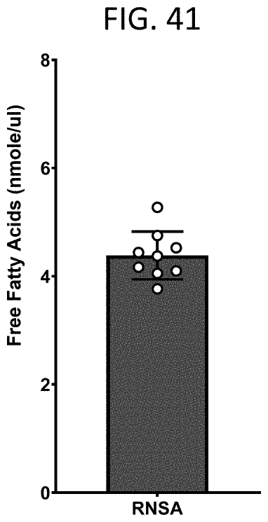

The contents of the inventive RNSA can be generally be regarded as having a protein component and a lipid component, which lipids were quantified after production as total free fatty acids (FFA). FFA quantification was performed on vials of manufactured samples (N=5) of the RNSA composition according to the invention. Analysis was performed by directly measuring the contents of FFA in each vial according to the manufacturer's instructions (see Abcam ab242305). Results of FFA quantitation are shown in FIG. 41.

Results indicate that the manufacturing methodology gives a consistent product yield with respect to FFA content across five different lots.

### Example 11: Human Tendinopathy Trial for Evaluation of the Effect of Cell-Free Regenerative Nonsteroidal Anti-inflammatory (RNSA) Compositions

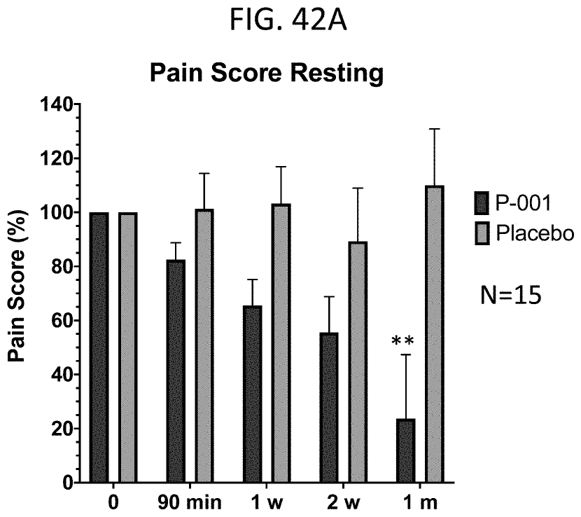

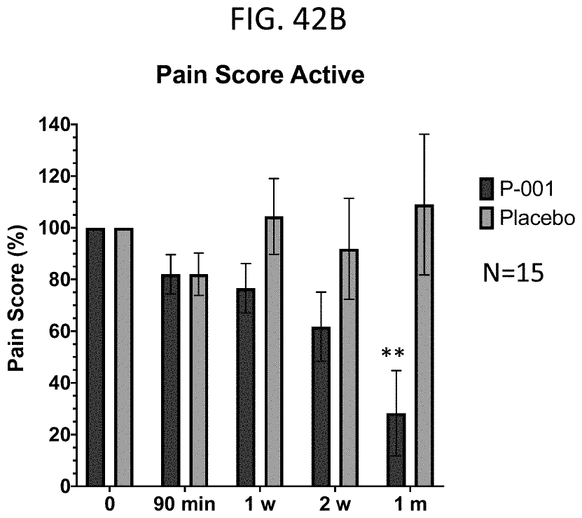

In a double blind placebo-controlled, IRB approved human trial, human patients (N=15) having tendinopathy of both elbows were treated by injection into the tendon with a single dose (4 μmol of FFA per dosage) of an exemplary RNSA composition (e.g., P-001) according to the invention at the site of tendinopathy in the first elbow. Patients were treated with an injection of saline in the second elbow. Pain scores during resting and active states were taken over the course of one month using standard visual analog scoring. Data was normalized, compiled, and represented in graphical form in FIGS. 42A and 42B.

Results indicate a significant decrease in pain scores for RNSA treated elbow tendinopathy compared to placebo, which began as early as 90 min post injection. Pain continued to reduce over 30 days, indicating long-term effect from a single injection. These results provide initial proof of concept that treatment with the inventive RNSA is associated with pain reduction, nociceptive pain reduction, anti-inflammatory effects, and regenerative effects in tendinopathy patients.

These results demonstrate the potential of the inventive RNSA for treating soft tissue injury/inflammation and perhaps other indications for which corticosteroids are currently used as treatment. Without being bound by theory, it has been proposed that the prolonged therapeutic effect of the inventive RNSA, which has a short half-life in vivo, induces the recipient's immune system to reprogram its response to chronically present local antigens. This reprogramming may promote long term immune tolerance of said antigens by regulating effector T cells and promoting regulatory T cells.

### Example 12: Human Pharmacokinetic/Pharmacodynamic Study for Evaluation of Cell-Free Regenerative Nonsteroidal Anti-Inflammatory (RNSA) Compositions

A pharmacokinetic and pharmacodynamic study was performed on a single human subject. The human subject fasted for 8 hours before infusion and during the entirety of the study (24 h). A peripheral intravenous (IV) bolus of RNSA (32 μmol/18 mL) was administered by infusion. The infusion was done using a 21G catheter directly connected to a syringe and slowly pushed. Blood samples (8 ml) were collected from the opposite arm at the following time intervals:
Sample 1: RNSA composition;
Sample 2: time=−1; immediately before infusion;
Sample 3: time=0; immediately after infusion
Sample 4: time=15 minutes after infusion
Sample 5: time=30 minutes after infusion
Sample 6: time=45 minutes after infusion
Sample 7: time=60 minutes after infusion
Sample 8: time=90 minutes after infusion
Sample 9: time=120 minutes after infusion
Sample 10: time=180 minutes after infusion
Sample 11: time=300 minutes after infusion
Sample 12: time=480 minutes after infusion
Sample 13: time=720 minutes after infusion
Sample 14: time=1440 minutes after infusion

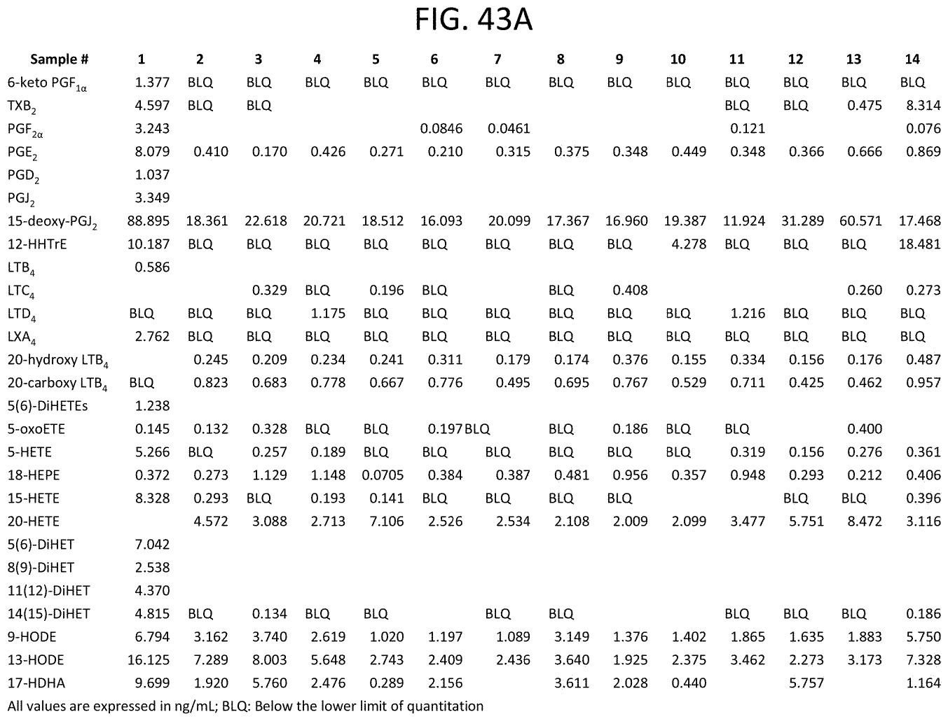

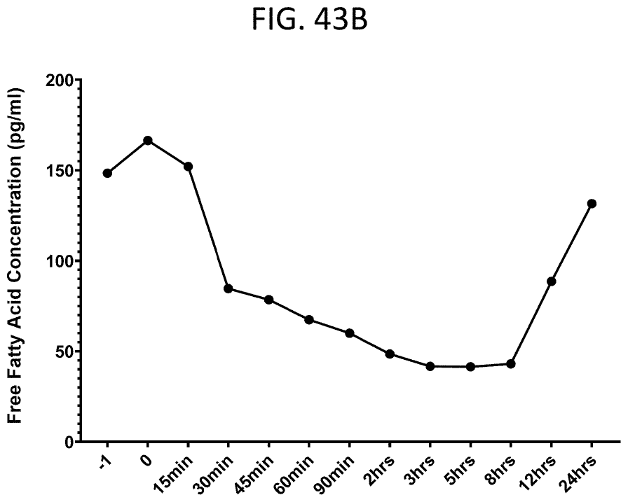

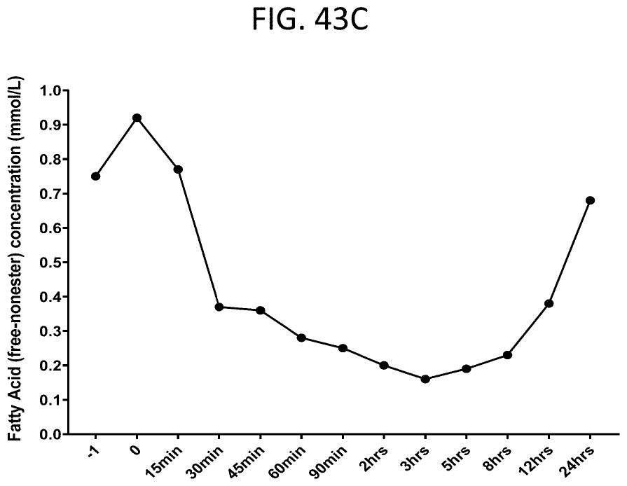

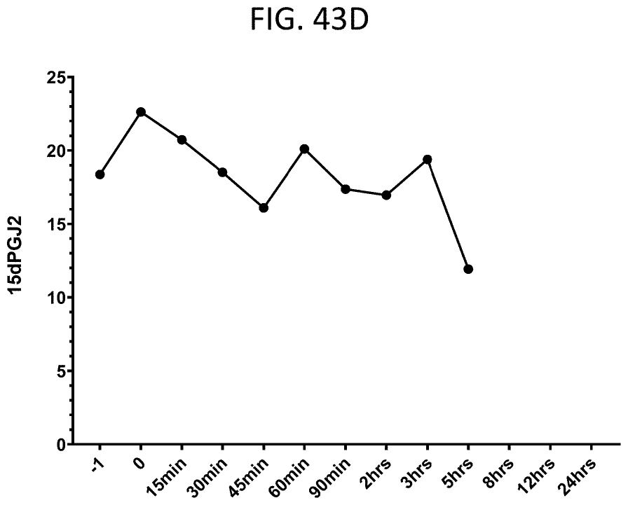

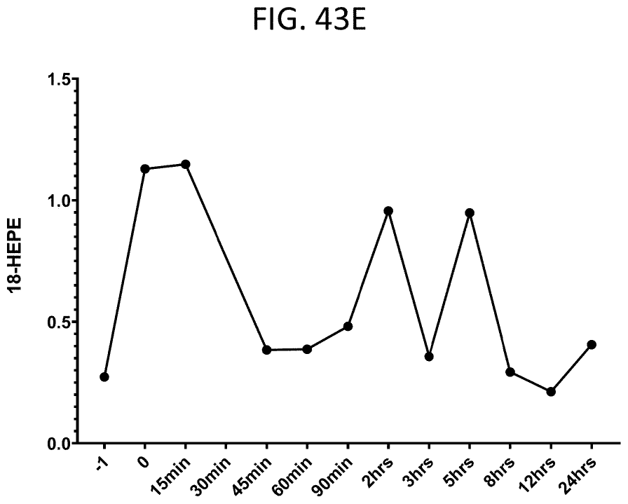

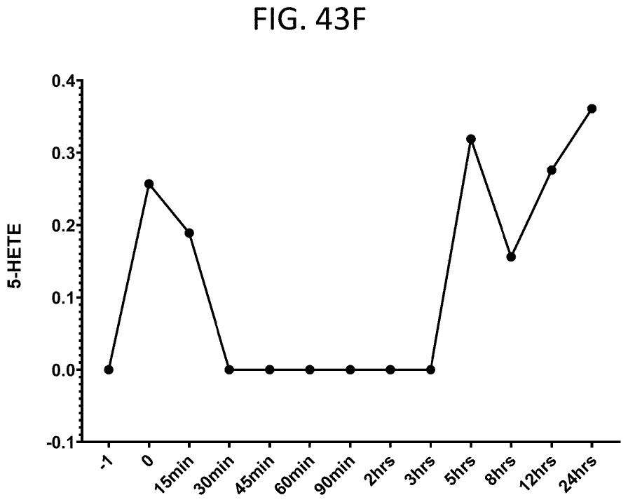

Blood samples were collected in purple top vials (EDTA K2) and centrifuged (1000× for 10 min). After blood components separated, plasma was aspirated and collected in microcentrifuge tubes (1 mL each) and stored at −80° C. until further analysis. Total FFA quantification was performed on plasma samples using two different methods of analysis. Total FFA was determined as detailed in Example 10 and by standard LabCorp protocols. Plasma samples were sent to Cayman Chemical for HPLC Purification Service and LC-MS Sample Analysis for an Eicosanoid/Oxylipin Biomarker Profiling. Biomarkers were quantified using calibration curves for authentic standards or structurally related surrogates. The entire Eicosanoid/Oxylipin Biomarker Profile is shown in FIG. 43A and graphs showing concentration of total FFA and selected biomarkers are shown in FIGS. 43B-F.

Results indicate that the inventive RNSA has a systemic effect on various lipid hormones in the body and induces pharmacodynamic effects pertinent to fatty acid regulation. Total FFA levels initially increase due to the FFA content in RNSA composition and then decrease over a period of 8 hours, followed by a return to baseline level.

These results suggest that the inventive RNSA activates mechanisms of fatty acid mobilization and has physio-modulatory effects on lipid transport and vascular physiology. These results further demonstrate the regulatory effects if RNSA and potential therapeutic targets pertinent to inflammation and elevated FFA (e.g., pulmonary fibrosis, type 2 diabetes, cardiovascular diseases, endothelial disfunction, metabolic syndrome, fatty liver (NASH), Alzheimer's and other diseases associated with brain inflammation and neurodegeneration, psoriasis, chronic inflammatory diseases including rheumatoid arthritis and asthma, and Alström syndrome).

These results provide initial proof of concept that the RNSA has potential therapeutic effects on conditions in which lipids are involved and on conditions in which prostaglandins, eicosanoids, inflammation are dysregulated. No adverse health effects were reported, providing initial indication that the inventive RNSA is safe to use systemically.

### Example 13: Evaluation of Long Term Stability of Cell-Free Regenerative Nonsteroidal Anti-Inflammatory (RNSA) Compositions

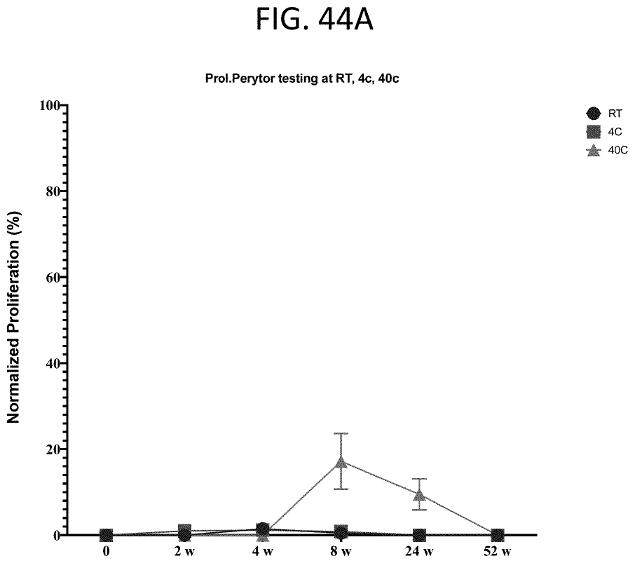

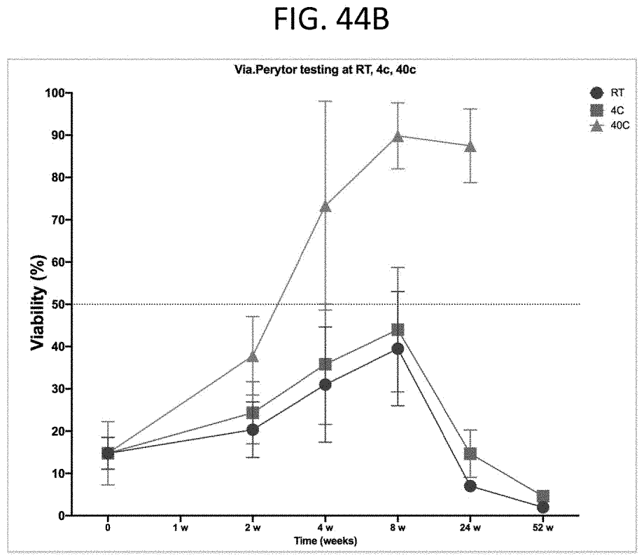

The effect of the inventive RNSA on inhibiting T cell proliferation and promoting apoptosis on T-effector cells was assessed at various timepoints during storage for 52 weeks at room temperature (RT), 4° C. and 40° C. (accelerated). The same human PBMC/T cells activation methodology was employed as described in Example 3. Results for percent proliferation of T cells and percent viability of T-effector cells are shown in FIGS. 44A and 44B, respectively.

Results indicate that the inventive RNSA composition displays prolonged stability and enhanced bioactivity after storage at room temperature and 4° C. Heating the RNSA composition to 40° C. results in prolonged stability and further enhancement of bioactivity.

These results demonstrate that the inventive RNSA composition retains bioactivity in a vial for final delivery to patient with or without excipients. Additionally, the current method of manufacture renders the composition bioavailable and bioactive (in vitro) when stored on a closed vial for later clinical use.

Without being bound to theory, it has been proposed that increased bioactivity may result from degradation of interfering molecules which are initially present in the composition after manufacturing.

### Example 14: Characterization of T-Effector Regulation by Cell-Free Regenerative Nonsteroidal Anti-Inflammatory (RNSA) Compositions

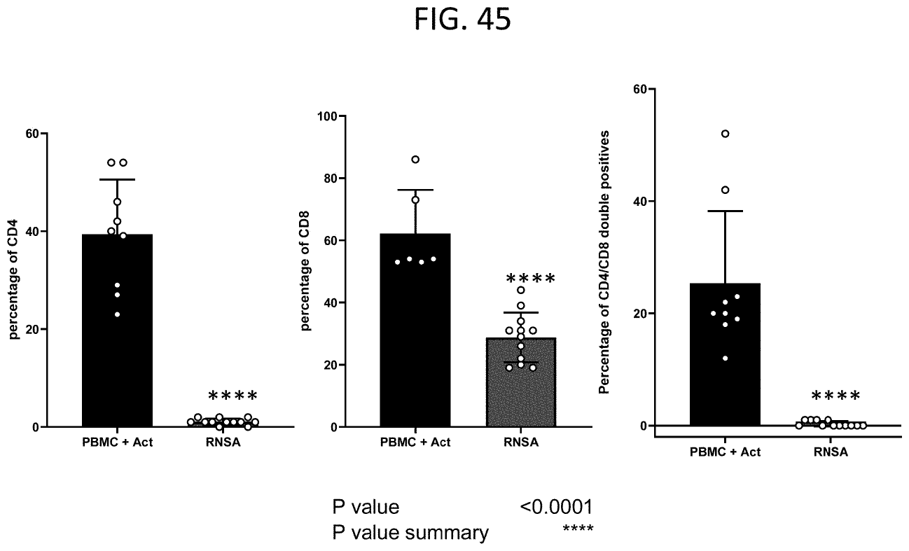

Regulatory effects of the inventive RNSA composition on human T-effector cells were characterized. The same human PBMC/T cells activation methodology was employed as described in Example 3. Results are shown in FIG. 45.

Results indicate downregulation of CD4, CD8, and CD4/CD8 double positive cells following treatment with RNSA. These results demonstrate that the inventive RNSA composition modulates potential therapeutic targets pertinent to T lymphoblastic leukemia/lymphoma (T-ALL), T-cell prolymphocytic leukemia (T-PLL), infiltrating T cells in nodular lymphocyte predominant Hodgkin lymphoma (NLPHL), autoimmune diseases, such as myasthenia gravis, rheumatoid arthritis, and multiple sclerosis, and other chronic inflammatory conditions. In addition, this population has been described in the inflammatory infiltrate of tumors, such as in nodular lymphocyte-predominant Hodgkin lymphoma. Additional potential patient populations for RNSA therapy include Chagasic patients with double-positive T Cells and patients with chronic cytokine and cytokine storm due to viral diseases or treatments (e.g., anticancer cell therapies).

### Example 15: Proteomic Analysis of RNSA Composition

The contents of the inventive RNSA can be generally be regarded as having a protein component and a lipid component, which proteins were analyzed after production by liquid chromatography-mass spectrometry (LC-MS). An exemplary RNSA composition according to the invention was subjected to proteomic analysis by Cayman Chemical via partner company MS Bioworks.

A 300 μL of sample of RNSA (<3 kDa fraction) was passed over an Amicon Ultra 20 kD MWCO spin cartridge. The flow-through was acidified and subjected to solid phase extraction on a Waters μHLB C18 plate. The eluate was reconstituted in 0.1% TFA for analysis.

Peptides (100% per sample) were analyzed by nano LC-MS/MS using a Waters NanoAcquity system interfaced to a Thermo Fisher Fusion Lumos mass spectrometer. Peptides were loaded on a trapping column and eluted over a 75 μm analytical column at 350 nL/min; both columns were packed with XSelect CSH C18 resin (Waters). A 1 hour gradient was employed. The mass spectrometer was operated in data-dependent mode, with MS and MS/MS performed in the Orbitrap at 60,000 FWHM resolution and 15,000 FWHM resolution, respectively. APD was turned on. The instrument was run with a 3 second cycle for MS and MS/MS.

Data were searched using a local copy of Byonic with the following parameters: Enzyme: None; Database: Swissprot Human (concatenated forward and reverse plus common contaminants); Fixed modification: None; Variable modifications: Oxidation (M), Acetyl (Protein N-terminus), Deamidation (NQ); Mass values: Monoisotopic; Peptide Mass Tolerance: 10 ppm; Fragment Mass Tolerance: 20 ppm; Max Missed Cleavages: 2.

Byonic mzID files were parsed into the Scaffold software for validation, filtering and to create a nonredundant list per sample. Data were filtered at 1% protein and peptide level FDR and requiring at least one unique peptide per protein. Search results listing peptides identified in the RNSA sample and their spectral counts (SpC) are provided separately as an appendix. Search results of identified proteins and their spectral counts (SpC) are listed in Table 1.

TABLE 1
Proteomic content of RNSA composition

| Identified Proteins (102) | Accession No. | Alt. ID | MW | | SpC |
|---|---|---|---|---|---|
| Annexin A2 OS = Homo sapiens | P07355 | ANXA2 | 39 | kDa | 132 |
| OX = 9606 GN = ANXA2 PE = 1 SV = 2 | | | | | |
| Neuroblast differentiation- | Q09666 | AHNAK | 629 | kDa | 30 |
| associated protein AHNAK | | | | | |
| OS = Homo sapiens OX = 9606 | | | | | |
| GN = AHNAK PE = 1 SV = 2 | | | | | |
| Decorin OS = Homo sapiens OX = 9606 | P07585 | DCN | 40 | kDa | 27 |
| GN = DCN PE = 1 SV = 1 | | | | | |
| Actin, cytoplasmic 1 OS = Homo | P60709 | ACTB | 42 | kDa | 30 |
| sapiens OX = 9606 GN = ACTB PE = 1 | | | | | |
| SV = 1 | | | | | |
| Mimecan OS = Homo sapiens | P20774 | OGN | 34 | kDa | 18 |
| OX = 9606 GN = OGN PE = 1 SV = 1 | | | | | |
| Filamin-B OS = Homo sapiens | O75369 | FLNB | 278 | kDa | 7 |
| OX = 9606 GN = FLNB PE = 1 SV = 2 | | | | | |
| L-lactate dehydrogenase A chain | P00338 | LDHA | 37 | kDa | 5 |
| OS = Homo sapiens OX = 9606 | | | | | |
| GN = LDHA PE = 1 SV = 2 | | | | | |
| Gelsolin OS = Homo sapiens OX = 9606 | P06396 | GSN | 86 | kDa | 9 |
| GN = GSN PE = 1 SV = 1 | | | | | |
| Annexin A1 OS = Homo sapiens | P04083 | ANXA1 | 39 | kDa | 6 |
| OX = 9606 GN = ANXA1 PE = 1 SV = 2 | | | | | |
| Protein AHNAK2 OS = Homo sapiens | Q8IVF2 | AHNAK2 | 617 | kDa | 1 |
| OX = 9606 GN = AHNAK2 PE = 1 SV = 2 | | | | | |
| Calmodulin-1 OS = Homo sapiens | P0DP23 (+2) | CALM1 | 17 | kDa | 8 |
| OX = 9606 GN = CALM1 PE = 1 SV = 1 | | | | | |
| Myosin-14 OS = Homo sapiens | Q7Z406 | MYH14 | 228 | kDa | 10 |
| OX = 9606 GN = MYH14 PE = 1 SV = 2 | | | | | |
| Ezrin OS = Homo sapiens OX = 9606 | P15311 | EZR | 69 | kDa | 3 |
| GN = EZR PE = 1 SV = 4 | | | | | |
| Thrombospondin-1 OS = Homo | P07996 | THBS1 | 129 | kDa | 6 |
| sapiens OX = 9606 GN = THBS1 PE = 1 | | | | | |
| SV = 2 | | | | | |
| Galectin-3 OS = Homo sapiens | P17931 | LGALS3 | 26 | kDa | 8 |
| OX = 9606 GN = LGALS3 PE = 1 SV = 5 | | | | | |
| Non-histone chromosomal protein | P05204 | HMGN2 | 9 | kDa | 5 |
| HMG-17 OS = Homo sapiens | | | | | |
| OX = 9606 GN = HMGN2 PE = 1 SV = 3 | | | | | |
| Lumican OS = Homo sapiens | P51884 | LUM | 38 | kDa | 5 |
| OX = 9606 GN = LUM PE = 1 SV = 2 | | | | | |
| Prolargin OS = Homo sapiens | P51888 | PRELP | 44 | kDa | 6 |
| OX = 9606 GN = PRELP PE = 1 SV = 1 | | | | | |
| Mucin-like protein 1 OS = Homo | Q96DR8 | MUCL1 | 9 | kDa | 3 |
| sapiens OX = 9606 GN = MUCL1 PE = 1 | | | | | |
| SV = 1 | | | | | |
| Vesicle-associated membrane | Q9P0L0 | VAPA | 28 | kDa | 4 |
| protein-associated protein A | | | | | |
| OS = Homo sapiens OX = 9606 | | | | | |
| GN = VAPA PE = 1 SV = 3 | | | | | |
| Protein S100-A9 OS = Homo sapiens | P06702 | S100A9 | 13 | kDa | 4 |
| OX = 9606 GN = S100A9 PE = 1 SV = 1 | | | | | |
| Uroplakin-3b-like protein 1 | B0FP48 | UPK3BL1 | 28 | kDa | 5 |
| OS = Homo sapiens OX = 9606 | | | | | |
| GN = UPK3BL1 PE = 2 SV = 1 | | | | | |
| Protein S100-A6 OS = Homo sapiens | P06703 | S100A6 | 10 | kDa | 5 |
| OX = 9606 GN = S100A6 PE = 1 SV = 1 | | | | | |
| Histone H1.3 OS = Homo sapiens | P16402 | H1-3 | 22 | kDa | 6 |
| OX = 9606 GN = H1-3 PE = 1 SV = 2 | | | | | |
| Protein S100-A4 OS = Homo sapiens | P26447 | S100A4 | 12 | kDa | 6 |
| OX = 9606 GN = S100A4 PE = 1 SV = 1 | | | | | |
| Solute carrier family 2, facilitated | P11166 | SLC2A1 | 54 | kDa | 4 |
| glucose transporter member 1 | | | | | |
| OS = Homo sapiens OX = 9606 | | | | | |
| GN = SLC2A1 PE = 1 SV = 2 | | | | | |
| Protein MAL2 OS = Homo sapiens | Q969L2 | MAL2 | 19 | kDa | 4 |
| OX = 9606 GN = MAL2 PE = 1 SV = 1 | | | | | |
| MARVEL domain-containing protein | Q9BSK0 | MARVELD1 | 19 | kDa | 1 |
| 1 OS = Homo sapiens OX = 9606 | | | | | |
| GN = MARVELD1 PE = 1 SV = 1 | | | | | |
| Protein S100-A10 OS = Homo sapiens | P60903 | S100A10 | 11 | kDa | 3 |
| OX = 9606 GN = S100A10 PE = 1 SV = 2 | | | | | |
| Epiplakin OS = Homo sapiens | P58107 | EPPK1 | 556 | kDa | 4 |
| OX = 9606 GN = EPPK1 PE = 1 SV = 3 | | | | | |
| Dihydropyrimidinase-related | Q16555 | DPYSL2 | 62 | kDa | 4 |
| protein 2 OS = Homo sapiens | | | | | |
| OX = 9606 GN = DPYSL2 PE = 1 SV = 1 | | | | | |
| Protein FAM98B OS = Homo sapiens | Q52LJ0 | FAM98B | 46 | kDa | 2 |
| OX = 9606 GN = FAM98B PE = 1 SV = 2 | | | | | |
| PRA1 family protein 2 OS = Homo | O60831 | PRAF2 | 19 | kDa | 5 |
| sapiens OX = 9606 GN = PRAF2 PE = 1 | | | | | |
| SV = 1 | | | | | |
| Plectin OS = Homo sapiens OX = 9606 | Q15149 | PLEC | 532 | kDa | 3 |
| GN = PLEC PE = 1 SV = 3 | | | | | |
| 14-3-3 protein sigma OS = Homo | P31947 | SFN | 28 | kDa | 3 |
| sapiens OX = 9606 GN = SFN PE = 1 | | | | | |
| SV = 1 | | | | | |
| Collagen alpha-3(VI) chain | P12111 | COL6A3 | 344 | kDa | 1 |
| OS = Homo sapiens OX = 9606 | | | | | |
| GN = COL6A3 PE = 1 SV = 5 | | | | | |
| Endoplasmic reticulum chaperone | P11021 | HSPA5 | 72 | kDa | 4 |
| BiP OS = Homo sapiens OX = 9606 | | | | | |
| GN = HSPA5 PE = 1 SV = 2 | | | | | |
| Hemoglobin subunit beta OS = Homo | P68871 (+1) | HBB | 16 | kDa | 3 |
| sapiens OX = 9606 GN = HBB PE = 1 | | | | | |
| SV = 2 | | | | | |
| Protein disulfide-isomerase A3 | P30101 | PDIA3 | 57 | kDa | 3 |
| OS = Homo sapiens OX = 9606 | | | | | |
| GN = PDIA3 PE = 1 SV = 4 | | | | | |
| Alpha-actinin-4 OS = Homo sapiens | O43707 | ACTN4 | 105 | kDa | 1 |
| OX = 9606 GN = ACTN4 PE = 1 SV = 2 | | | | | |
| Tubulin beta chain OS = Homo | P07437 | TUBB | 50 | kDa | 1 |
| sapiens OX = 9606 GN = TUBB PE = 1 | | | | | |
| SV = 2 | | | | | |
| Fibrinogen alpha chain OS = Homo | P02671 | FGA | 95 | kDa | 1 |
| sapiens OX = 9606 GN = FGA PE = 1 | | | | | |
| SV = 2 | | | | | |
| Membrane-associated | O00264 | PGRMC1 | 22 | kDa | 1 |
| progesterone receptor component | | | | | |
| 1 OS = Homo sapiens OX = 9606 | | | | | |
| GN = PGRMC1 PE = 1 SV = 3 | | | | | |
| 60S ribosomal protein L7a | P62424 | RPL7A | 30 | kDa | 2 |
| OS = Homo sapiens OX = 9606 | | | | | |
| GN = RPL7A PE = 1 SV = 2 | | | | | |
| Transforming growth factor-beta- | Q15582 | TGFBI | 75 | kDa | 2 |
| induced protein ig-h3 OS = Homo | | | | | |
| sapiens OX = 9606 GN = TGFBI PE = 1 | | | | | |
| SV = 1 | | | | | |
| Serpin B3 OS = Homo sapiens | P29508 (+1) | SERPINB3 | 45 | kDa | 2 |
| OX = 9606 GN = SERPINB3 PE = 1 SV = 2 | | | | | |
| Cadherin-1 OS = Homo sapiens | P12830 | CDH1 | 97 | kDa | 1 |
| OX = 9606 GN = CDH1 PE = 1 SV = 3 | | | | | |
| Envoplakin OS = Homo sapiens | Q92817 | EVPL | 232 | kDa | 3 |
| OX = 9606 GN = EVPL PE = 1 SV = 3 | | | | | |
| Putative small nuclear | A8MWD9 | SNRPGP15 | 9 | kDa | 3 |
| ribonucleoprotein G-like protein 15 | (+1) | | | | |
| OS = Homo sapiens OX = 9606 | | | | | |
| GN = SNRPGP15 PE = 5 SV = 2 | | | | | |
| Heat shock protein HSP 90-alpha | P07900 | HSP90AA1 | 85 | kDa | 3 |
| OS = Homo sapiens OX = 9606 | | | | | |
| GN = HSP9OAA1 PE = 1 SV = 5 | | | | | |
| Alpha-enolase OS = Homo sapiens | P06733 | ENO1 | 47 | kDa | 1 |
| OX = 9606 GN = ENO1 PE = 1 SV = 2 | | | | | |
| Clathrin heavy chain 1 OS = Homo | Q00610 | CLTC | 192 | kDa | 1 |
| sapiens OX = 9606 GN = CLTC PE = 1 | | | | | |
| SV = 5 | | | | | |
| Histone H1.4 OS = Homo sapiens | P10412 | H1-4 | 22 | kDa | 1 |
| OX = 9606 GN = H1-4 PE = 1 SV = 2 | | | | | |
| Thioredoxin domain-containing | O95881 | TXNDC12 | 19 | kDa | 2 |
| protein 12 OS = Homo sapiens | | | | | |
| OX = 9606 GN = TXNDC12 PE = 1 SV = 1 | | | | | |
| Protein S100-A7 OS = Homo sapiens | P31151 (+1) | S100A7 | 11 | kDa | 2 |
| OX = 9606 GN = S100A7 PE = 1 SV = 4 | | | | | |
| Tubulin alpha-lC chain OS = Homo | Q9BQE3 | TUBA1C | 50 | kDa | 2 |
| sapiens OX = 9606 GN = TUBA1C PE = 1 | | | | | |
| SV = 1 | | | | | |
| CD109 antigen OS = Homo sapiens | Q6YHK3 | CD109 | 162 | kDa | 1 |
| OX = 9606 GN = CD109 PE = 1 SV = 2 | | | | | |
| Reticulon-4 OS = Homo sapiens | Q9NQC3 | RTN4 | 130 | kDa | 1 |
| OX = 9606 GN = RTN4 PE = 1 SV = 2 | | | | | |
| Protein RER1 OS = Homo sapiens | O15258 | RER1 | 23 | kDa | 2 |
| OX = 9606 GN = RER1 PE = 1 SV = 1 | | | | | |
| Glyceraldehyde-3-phosphate | P04406 | GAPDH | 36 | kDa | 1 |
| dehydrogenase OS = Homo sapiens | | | | | |
| OX = 9606 GN = GAPDH PE = 1 SV = 3 | | | | | |
| Tenascin-X OS = Homo sapiens | P22105 | TNXB | 458 | kDa | 2 |
| OX = 9606 GN = TNXB PE = 1 SV = 5 | | | | | |
| Keratin, type II cytoskeletal 6A | P02538 (+1) | KRT6A | 60 | kDa | 1 |
| OS = Homo sapiens OX = 9606 | | | | | |
| GN = KRT6A PE = 1 SV = 3 | | | | | |
| ATP synthase subunit beta, | P06576 | ATP5F1B | 57 | kDa | 2 |
| mitochondrial OS = Homo sapiens | | | | | |
| OX = 9606 GN = ATP5F1B PE = 1 SV = 3 | | | | | |
| Synaptic vesicle membrane protein | Q99536 | VAT1 | 42 | kDa | 2 |
| VAT-1 homolog OS = Homo sapiens | | | | | |
| OX = 9606 GN = VAT1 PE = 1 SV = 2 | | | | | |
| 14-3-3 protein epsilon OS = Homo | P62258 | YWHAE | 29 | kDa | 2 |
| sapiens OX = 9606 GN = YWHAE PE = 1 | | | | | |
| SV = 1 | | | | | |
| DnaJ homolog subfamily B member | P25685 | DNAJB1 | 38 | kDa | 2 |
| 1 OS = Homo sapiens OX = 9606 | | | | | |
| GN = DNAJB1 PE = 1 SV = 4 | | | | | |
| Shootin-1 OS = Homo sapiens | A0MZ66 | SHTN1 | 72 | kDa | 1 |
| OX = 9606 GN = SHTN1 PE = 1 SV = 4 | | | | | |
| Proteolipid protein 2 OS = Homo | Q04941 | PLP2 | 17 | kDa | 1 |
| sapiens OX = 9606 GN = PLP2 PE = 1 | | | | | |
| SV = 1 | | | | | |
| T-cell surface glycoprotein CD3 | P04234 | CD3D | 19 | kDa | 1 |
| delta chain OS = Homo sapiens | | | | | |
| OX = 9606 GN = CD3D PE = 1 SV = 1 | | | | | |
| Basic salivary proline-rich protein 2 | P02812 | PRB2 | 41 | kDa | 2 |
| OS = Homo sapiens OX = 9606 | | | | | |
| GN = PRB2 PE = 1 SV = 3 | | | | | |
| Voltage-dependent anion-selective | P21796 | VDAC1 | 31 | kDa | 2 |
| channel protein 1 OS = Homo sapiens | | | | | |
| OX = 9606 GN = VDAC1 PE = 1 SV = 2 | | | | | |
| Protein S100-A8 OS = Homo sapiens | P05109 | S100A8 | 11 | kDa | 1 |
| OX = 9606 GN = S100A8 PE = 1 SV = 1 | | | | | |
| Adseverin OS = Homo sapiens | Q9Y6U3 | SCIN | 80 | kDa | 1 |
| OX = 9606 GN = SCIN PE = 1 SV = 4 | | | | | |
| Alpha-crystallin B chain OS = Homo | P02511 | CRYAB | 20 | kDa | 1 |
| sapiens OX = 9606 GN = CRYAB PE = 1 | | | | | |
| SV = 2 | | | | | |
| Thymosin beta-4 OS = Homo sapiens | P62328 | TMSB4X | 5 | kDa | 2 |
| OX = 9606 GN = TMSB4X PE = 1 SV = 2 | | | | | |
| Histone H2B type 1-K OS = Homo | O60814 (+4) | H2BC12 | 14 | kDa | 1 |
| sapiens OX = 9606 GN = H2BC12 PE = 1 | | | | | |
| SV = 3 | | | | | |
| Apolipoprotein A-I OS = Homo | P02647 | APOA1 | 31 | kDa | 1 |
| sapiens OX = 9606 GN = APOA1 PE = 1 | | | | | |
| SV = 1 | | | | | |
| Perilipin-3 OS = Homo sapiens | O60664 | PLIN3 | 47 | kDa | 1 |
| OX = 9606 GN = PLIN3 PE = 1 SV = 3 | | | | | |
| Ubiquitin-conjugating enzyme E2 | Q15819 | UBE2V2 | 16 | kDa | 2 |
| variant 2 OS = Homo sapiens | | | | | |
| OX = 9606 GN = UBE2V2 PE = 1 SV = 4 | | | | | |
| Heterogeneous nuclear | P51991 | HNRNPA3 | 40 | kDa | 1 |
| ribonucleoprotein A3 OS = Homo | | | | | |
| sapiens OX = 9606 GN = HNRNPA3 | | | | | |
| PE = 1 SV = 2 | | | | | |
| Histone H1.0 OS = Homo sapiens | P07305 | H1-0 | 21 | kDa | 2 |
| OX = 9606 GN = H1-0 PE = 1 SV = 3 | | | | | |
| Tropomodulin-3 OS = Homo sapiens | Q9NYL9 | TMOD3 | 40 | kDa | 1 |
| OX = 9606 GN = TMOD3 PE = 1 SV = 1 | | | | | |
| Sideroflexin-1 OS = Homo sapiens | Q9H9B4 | SFXN1 | 36 | kDa | 1 |
| OX = 9606 GN = SFXN1 PE = 1 SV = 4 | | | | | |
| Fibronectin OS = Homo sapiens | P02751 | FN1 | 272 | kDa | 1 |
| OX = 9606 GN = FN1 PE = 1 SV = 5 | | | | | |
| Golgi resident protein GCP60 | Q9H3P7 | ACBD3 | 61 | kDa | 1 |
| OS = Homo sapiens OX = 9606 | | | | | |
| GN = ACBD3 PE = 1 SV = 4 | | | | | |
| Galectin-7 OS = Homo sapiens | P47929 | LGALS7 | 15 | kDa | 1 |
| OX = 9606 GN = LGALS7 PE = 1 SV = 2 | | | | | |
| Citrate synthase, mitochondrial | O75390 | CS | 52 | kDa | 1 |
| OS = Homo sapiens OX = 9606 GN = CS | | | | | |
| PE = 1 SV = 2 | | | | | |
| Immunoglobulin heavy constant | P01857 (+3) | IGHG1 | 36 | kDa | 1 |
| gamma 1 OS = Homo sapiens | | | | | |
| OX = 9606 GN = IGHG1 PE = 1 SV = 1 | | | | | |
| Dihydropyrimidinase-related | Q14195 | DPYSL3 | 62 | kDa | 1 |
| protein 3 OS = Homo sapiens | | | | | |
| OX = 9606 GN = DPYSL3 PE = 1 SV = 1 | | | | | |
| Actin-related protein 2/3 complex | O15144 | ARPC2 | 34 | kDa | 1 |
| subunit 2 OS = Homo sapiens | | | | | |
| OX = 9606 GN = ARPC2 PE = 1 SV = 1 | | | | | |
| Glyoxylate | Q9UBQ7 | GRHPR | 36 | kDa | 1 |
| reductase/hydroxypyruvate | | | | | |
| reductase OS = Homo sapiens | | | | | |
| OX = 9606 GN = GRHPR PE = 1 SV = 1 | | | | | |
| Immunoglobulin heavy variable 3- | A0A0B4J1Y9 | IGHV3-72 | 13 | kDa | 1 |
| 72 OS = Homo sapiens OX = 9606 | | | | | |
| GN = IGHV3-72 PE = 3 SV = 1 | | | | | |
| UV excision repair protein RAD23 | P54727 | RAD23B | 43 | kDa | 1 |
| homolog B OS = Homo sapiens | | | | | |
| OX = 9606 GN = RAD23B PE = 1 SV = 1 | | | | | |
| Calpain small subunit 1 OS = Homo | P04632 | CAPNS1 | 28 | kDa | 1 |
| sapiens OX = 9606 GN = CAPNS1 PE = 1 | | | | | |
| SV = 1 | | | | | |
| Centrosomal protein of 112 kDa | Q8N8E3 | CEP112 | 113 | kDa | 1 |
| OS = Homo sapiens OX = 9606 | | | | | |
| GN = CEP112 PE = 1 SV = 2 | | | | | |
| 60 kDa heat shock protein, | P10809 | HSPD1 | 61 | kDa | 1 |
| mitochondrial OS = Homo sapiens | | | | | |
| OX = 9606 GN = HSPD1 PE = 1 SV = 2 | | | | | |
| Sodium- and chloride-dependent | P48029 | SLC6A8 | 71 | kDa | 1 |
| creatine transporter 1 OS = Homo | | | | | |
| sapiens OX = 9606 GN = SLC6A8 PE = 1 | | | | | |
| SV = 1 | | | | | |
| Fatty acid-binding protein 5 | Q01469 | FABP5 | 15 | kDa | 1 |
| OS = Homo sapiens OX = 9606 | | | | | |
| GN = FABP5 PE = 1 SV = 3 | | | | | |
| Microfibrillar-associated protein 2 | P55001 | MFAP2 | 21 | kDa | 1 |
| OS = Homo sapiens OX = 9606 | | | | | |
| GN = MFAP2 PE = 1 SV = 1 | | | | | |
| Cofilin-1 OS = Homo sapiens | P23528 | CFL1 | 19 | kDa | 1 |
| OX = 9606 GN = CFL1 PE = 1 SV = 3 | | | | | |
| Methyl-CpG-binding protein 2 | P51608 | MECP2 | 52 | kDa | 1 |
| OS = Homo sapiens OX = 9606 | | | | | |
| GN = MECP2 PE = 1 SV = 1 | | | | | |
| Serine/threonine-protein kinase | Q8IYT8 | ULK2 | 113 | kDa | 1 |
| ULK2 OS = Homo sapiens OX = 9606 | | | | | |
| GN = ULK2 PE = 1 SV = 3 | | | | | |

The data was further analyzed using PEAKS DB de novo sequencing assisted database search tool for peptide identification (Bioinformatics Solutions Inc.). Results for PEAKS DB and De Novo analysis are listed separately as an appendix.

Results indicate that the manufacturing methodology yields an RNSA composition comprising at least 296 detected peptides representative of at least 102 proteins, which are listed in Table 1, in addition to over 10,500 de novo peptide fragments (see Appendix) and the total FFA component described in Example 10.

### Example 16: RNSA Inhibits LPS Induced TNFα Response in PBMCs-Monocyte/Macrophage Model

Experimental Rationale:

Lipopolysaccharide is a known Escherichia coli endotoxin that stimulates pro-inflammatory cytokine response in monocytes and macrophages, as well as promoting M1 macrophage polarization via Toll-like Receptor 4 (TLR4) and monocyte autocrine response to LPS [Lawlor, N., et al., “Single Cell Analysis of Blood Mononuclear Cells Stimulated Through Either LPS or Anti-CD3 and Anti-CD28”, Front Immunol, 2021. 12: p. 636720; Orecchioni, M., et al., “Macrophage Polarization: Different Gene Signatures in M1(LPS+) vs. Classically and M2(LPS−) vs. Alternatively Activated Macrophages”, Front Immunol, 2019. 10: p. 1084. Recently, single-cell sequencing demonstrated that LPS treated PBMCs affects only the CD14+ monocyte lineage and induces transcriptional activation of Toll-like receptor, Nod-like receptor and NF-Kappa B pro-inflammatory pathways [Lawlor, N., et al., “Single Cell Analysis of Blood Mononuclear Cells Stimulated Through Either LPS or Anti-CD3 and Anti-CD28”, Front Immunol, 2021. 12: p. 636720]. Therefore, this model was used to probe the effect of RNSA or RNSA derived filtrates in ameliorating the monocyte-induced pro-inflammatory response to LPS.

Materials and Methods

Fresh leukoreduction system (LRS) cones of healthy donors aged 18-80 years old were purchased from Oklahoma Blood Institute. PBMCs were isolated from LRS cones using EasySep™ Direct Human PBMC Isolation Kit (cat #19654, StemCell Technologies) as per manufacturer's specifications. 1.5 million PBMCs were seeded in 0.5 ml of RPMI (11835030, Gibco) supplemented with 5% HPL (Millcreek), lx Glutamax (35050061, Gibco), 1×NEAA (11140050, Gibco) and 2 U/ml heparin (25021040030, Sagent Pharmaceuticals) in a 24 well plate and rested overnight. The following day, PBMCs were co-stimulated with 100 ng of lipopolysaccharide (LPS, 00-4976-93, Invitrogen) and different doses RNSA in a volume of 0.5 ml. RNSA doses were prepared in saline (R5201-01, Braun) supplemented with 5% HPL and 2 U/ml heparin. Betamethasone (Cayman, 20363), a positive control, was resuspended in DMSO (D4540, Sigma) and administered to PBMCs at different doses to a final 0.13% DMSO concentration. Vehicle for RNSA was 50% saline and for Betamethasone 0.13% DMSO for a 1 ml reaction. All samples were tested in technical triplicates. Treatment duration was 24 hours for all groups. After 24 hours, the supernatant was collected, spun at 5,000 g for 10 minutes and the cell free supernatant stored at −80 C. To detect TNFα, hTNFα Simple Plex assay quantification was performed on ELLA ELISA based detection method (ProteinSimple).

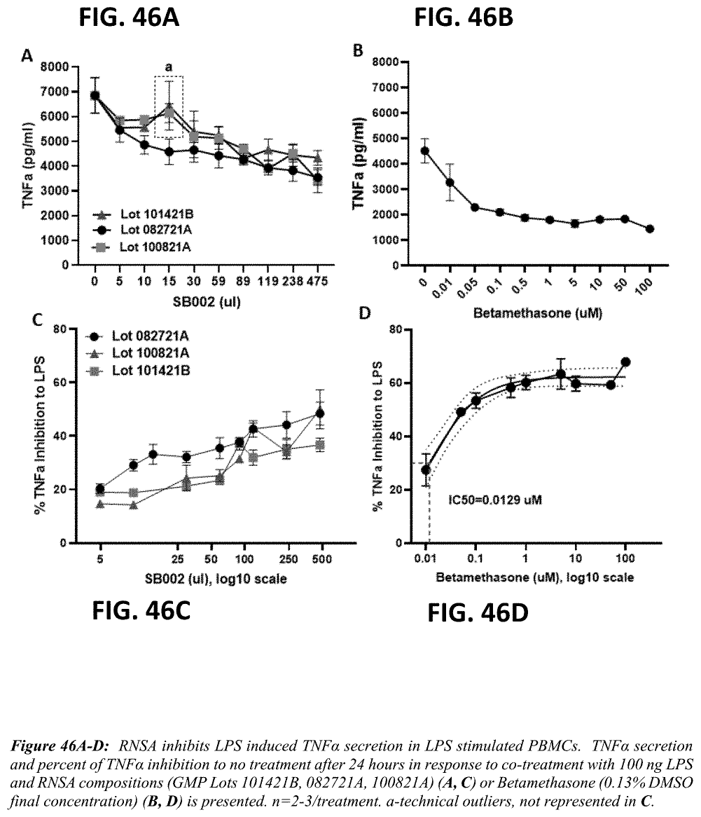

The results of these experiments are contained in FIG. 46A-D and show that RNSA inhibits LPS induced TNFα secretion in LPS stimulated PBMCs. In the experiments TNFα secretion and percent of TNFα inhibition was detected in the LPS-induced PBMC model after 24 hours after no treatment, co-treatment with 100 ng LPS and RNSA or RNSA derived filtrates (GMP Lots 101421B, 082721A, 100821A) (FIG. 46A, C) or co-treatment with 100 ng LPS and Betamethasone (0.13% DMSO final concentration) (FIGS. 46 B, D) is presented. n=2-3/treatment. a-technical outliers, not represented in FIG. 46C.

More specifically, in this LPS-induced PBMC model, TNFα secretion was robust reaching 6855±715 pg/ml after 24 hours of treatment (FIG. 46A). RNSA or RNSA filtrate treatment with doses ranging from 5 μl (1:200 dilution) to 475 μl (^{˜}1:2 dilution) inhibited TNFα secretion in a dose-response manner, with inhibitions ranging from 20-48% (Lot 082721A), 12-50% (Lot 100821A) and 19-37% (Lot 101421B) (FIG. 46C), for each lot respectively. In comparison, the glucocorticoid betamethasone attenuated TNFα response from 20-68% (0.01-100 μM) compared to vehicle control (0.13% DMSO), although TNFα secretion impacted by the DMSO presence (4512±476 pg/ml, no treatment) (FIG. 46B, D). Inhibition of TNFα by betamethasone in this model in a dose-dependent manner provides assurance of dose effect of RNSA, as well as the sensitivity of the model in determining this effect.

### Example 17: RNSA is a Strong Activator of PPARα and PPARγ

Experimental Rationale:

Experiments were conducted which demonstrated the effect of RNSA1 (082721A) and

RNSA2 (100821A) on PPARα and PPARγ. The results showed that both RNSA1 and RNSA2 exhibited dose-dependent stimulation of PPARα and PPARγ reporter activities (Single Factor ANOVA with p0.3). The actual effects by the RNSA samples are even larger than they appeared if corrected for the effect of saline vehicle controls above. Stimulatory effects of up to near 50% of maximal responses in PPARα and PPARγ reporter activation may be revealed after the correction.

Experimental Methods

Reporter assay kits for PPARα (Cat. #IB00111), PPARδ (Cat. #IB00121), PPARγ (Cat. #IB00101) were obtained from Indigo Biosciences. The reporter cells for PPARα, PPARδ, and PPARδ were seeded on the white 96-well opaque plates provided in the kit according to the kit booklets. The same cell suspensions were added onto a clear bottom 96-well tissue culture treated plate for monitoring cell attachment. The cells were fully attached to the surface within 2 hours of incubation at 37° C. with 5% CO2. Positive controls were serially diluted in CSM in half order and applied to the cells at 200 μl/well in duplicate. The RNSA samples were diluted in CSM and applied to the plates in triplicate at 200 μl/wells. For testing the effect of the saline vehicle solution, it was diluted in CSM to 2× of the final intended concentrations and pipetted 100 μl per well in two sets of duplicate wells. To one set of the duplicate wells with diluted saline solutions, 100 μl of CSM was added. To the other set of duplicate wells, 100 μl/well of 2× corresponding control agonists (19 nM GW7437 for PPARα, 6.32 nM GW0742 for PPARδ (“GW0742” which is also known as GW610742 is a PPARδ/β agonist developed by GlaxoSmithKline for use in treating metabolic disorders such as diabetes), and 600 nM Rosiglitazone for PPARγ) were added. Samples were examined for luciferase activity after 23 hours incubation at 37° C. with 5% CO2.

After 23 hours incubation, the media were discarded, and the plates were blotted upside down on paper towel. Detection substrate in buffer was prepared according to manufacturer and applied to the plates at 100 μl/well. After incubation for 5 minutes, the plates were scanned for luminescence in FlexStation 3 (Molecular Dynamics) with 500 ms integration per well followed a 5 second shaking. The plate scanning was repeated at 10 minutes and 15 minutes after addition of the substrate. The readings from 10 minutes incubation were used in the analysis. The dose-response curves were used to calculate the EC50 values using GraphPad Prism software. All data from sample treated wells were normalized to the maximal response of corresponding positive controls on the same plates.

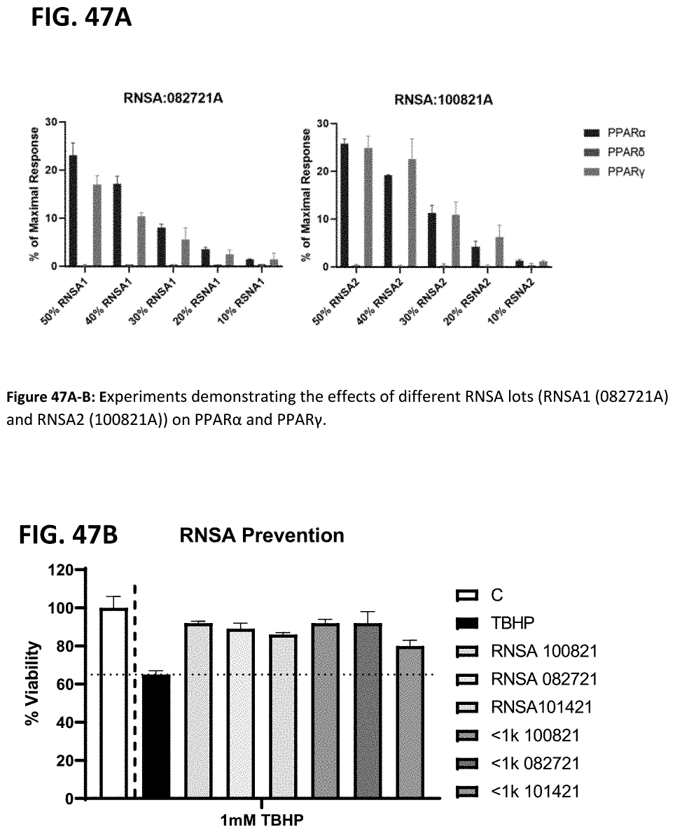

The results of these experiments are contained in FIG. 47A and FIG. 47B. As shown therein the secretomes RNSA1 (082721A) and RNSA2 (100821A) both triggered strong activations of PPARα and PPARγ reporters. The results of these experiments further demonstrate that the PPARδ reporter assay exhibited a dose-dependent response to the positive GW0742 control whereas none of the RNSA samples appeared to activate this pathway.

### Example 18: RNSA Treatment Prevents ROS Induced Apoptosis in Skin Fibroblasts

Experimental Rationale

tBHP is a known inducer of oxidative stress and has previously been used to induce cellular senescence in different types of cells (Dumont et al., “Induction of replicative senescence biomarkers by sublethal oxidative stresses in normal human fibroblast”, Free Radic Biol Med. 2000; 28:361-373; Kučera et al., “The Effect of tert-Butyl Hydroperoxide-Induced Oxidative Stress on Lean and Steatotic Rat Hepatocytes In Vitro”, Oxid Med Cell Longev. 2014; 2014:1-12. doi: 10.1155/2014/752506; Unterluggauer et al., “Senescence-associated cell death of human endothelial cells: the role of oxidative stress, Exp Gerontol. 2003; 38:1149-1160. doi: 10.1016/Jexger.2003.08.007). The chemical is known to deplete cellular antioxidant defense mechanisms (Crane et al., “Decreased Flux through Pyruvate Dehydrogenase by Thiol Oxidation during t-Butyl Hydroperoxide Metabolism in Perfused Rat Liver”, Hoppe-Seyler's Zeitschrift für Physiol Chemie. 1983; 364:977-988. doi: 10.1515/bchm2.1983.364.2.977; Zavodnik et al., “Activation of red blood cell glutathione peroxidase and morphological transformation of erythrocytes under the action of tert-butyl hydroperoxide”, IUBMB Life. 1998; 44:577-588. doi: 10.1080/15216549800201612), to produce radicals that initiate lipid peroxidation (Davies, “Detection of peroxyl and alkoxyl radicals produced by reaction of hydroperoxides with rat liver microsomal fractions”, Biochem J. 1989; 257:603-606. doi: 10.1042/bj2570603.) and to reduce mitochondrial membrane potential (ΔΨM) in neuronal cells (Wang et al., “Ethanol Extract of Centipeda minima Exerts Antioxidant and Neuroprotective Effects via Activation of the Nrf2 Signaling Pathway”, Oxid Med Cell Longev. 2019; 2019:1-16. doi: 10.1155/2019/9421037.)

Experimental Methods:

Skin fibroblasts from donors affected by Friedreich Ataxia (FA) were acquired from Coriell Institute (GM03816). Fibroblasts were seeded at 8,000 cells/well in 96 well plate 50 μl of αMEM (Gibco) supplemented with 30% FBS and 50 μl with either RNSA, <1 kDA RNSA fraction or saline (vehicle) (50% v/v treatment). The cells were cultured for 19 hours under standard incubator conditions. Afterwards, the cells were treated with 1 mM tert-Butyl hydroperoxide (TBHP, Invitrogen) for 5 hrs. CyQUANT XTT assay (Invitrogen) was utilized as per manufacturer's instruction to measure cell metabolic rate as a proxy measure for cell viability. Data presented as % viability normalized to saline control.

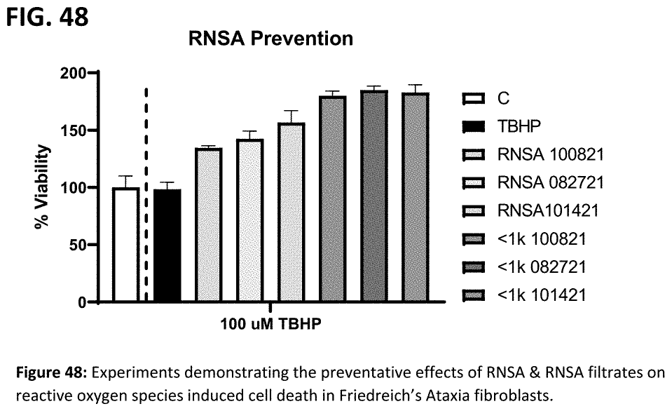

The <1kDA RNSA fraction was produced by centrifugation at 4° C. for 45 minutes of RNSA product in the Pall MCP001C41centrifugal filter. The results of these experiments are shown in FIG. 48. These results revealed that RNSA partially rescues Reactive Oxygen Species (ROS)-induced death in healthy fibroblasts and Friedreich's Ataxia patients. The data in FIG. 48 further shows the RNSA Cytoprotective/anti-ROS effects in the <1 KDa fractions.

### Example 19: RNSA Inhibition of Proliferation of Acute Lymphoblastic Leukemia Cells

Experiments were conducted to assess the effects of RNSA compositions obtained according to the methods disclosed herein on the proliferation of cancer cells (acute lymphoblastic leukemia cells). These experiments are described below.

Experimental Methods:

Jurkat cells, Clone E6-1 (TIB-152, lot 70044353) and ISO17034 CCRF-CEM (CRM-CCL-119, lot 70011017-013) were purchased from ATCC. The cells were cultured in RPMI 1640 ATCC formulation (A1049101, Gibco) supplemented with 10% FBS (10438034, Gibco) as per manufacturer's instruction for cell maintenance. For the assay development, Jurkat and CCRF-CEM T lymphoblasts were seeded at 75,000 cells/well in a 96 well plate, in 50 μl of media supplemented with 20% FBS. Afterwards, RPMI 1640 media alone (no treatment), 0.9% Sodium Chloride (saline, R5201, Braun) or RNSA (Signature Biologics) was added to the well at a volume of 50 μl, (50% v/v), for a total of 100 μl reaction volume. The cells were incubated for 48 hours, after which they were collected in a 0.5 ml Eppendorf tube. Via1-Cassette (Chemotec) was used to obtain the sample and run the cell count and viability on NC200 Nucleocounter (Chemotec). Cell number for each sample was recorded and expressed as a percent T cell change in proliferation compared to control saline (excipient).

For validation of the protocol with CCRF-CEM cells, the reaction was scaled up to a 48-well plate, where CCRF-CEM cells were seeded at 150,000 cells in 100 μl of 20% FBS media, and subsequently treated with 100 μl of the RPMI1640/excipient saline/RNSA for 24 or 48 hours. The cell count is determined as described above.

Results:

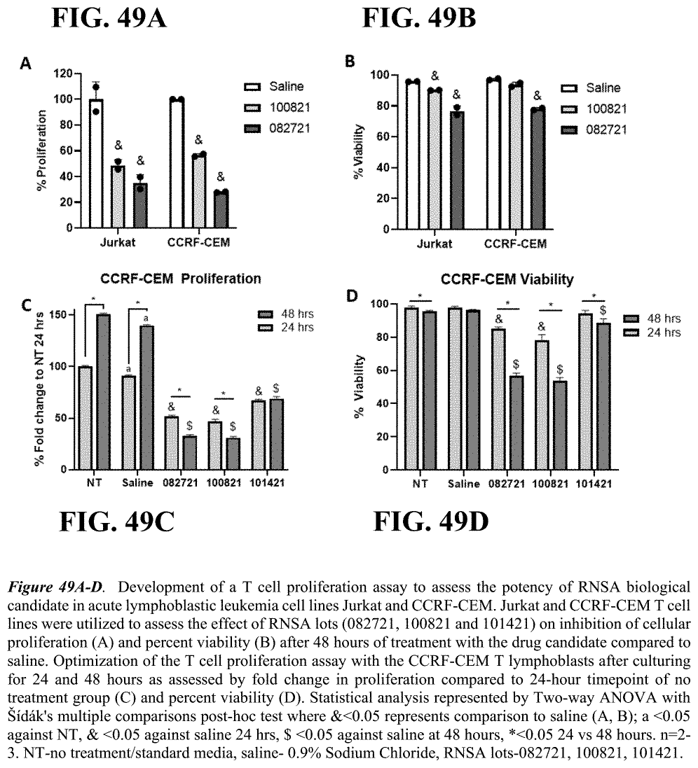

The assessment of RNSA potency on cellular proliferation in leukemic cell lines Jurkat and CCRF-CEM shows remarkably significant anti-proliferative and pro-apoptotic effects within 48 hours of treatment FIG. 49A & 49B). Two lots of RNSA (100821 and 082721) show statistically significant (p<0.05) effects on inhibition of cellular proliferation as depicted by a decrease in the population to an average of 51.3% and 65% in Jurkat T cells and 56.55% and 27.9% average proliferation in CCRF-CEM T cells, respectively with the two RNSA lots. The decrease in cell viability with RNSA treatment was found to be significant (p<0.05) for lot 100821 in both the cells lines and significant (p<0.05) in lot 082721 only with Jurkat T cell line, highlighting pro-apoptotic effects and potential cell line sensitivity to the drug candidate.

Based on the results herein described we chose to pursue further validation and refining of the potency assay with the CCRF-CEM cell line as American Type Culture Collection (ATCC) manufactures the cell lines under ISO 17034:2016 that qualifies the cell line as a reference material to be used in testing and calibration in 15017025 laboratories. The use of excipient solution saline as a control for the RNSA drug product demonstrate a decrease on average of 9% on proliferation that is statistically significant (p<0.05). This effect is most likely due to a decrease in the nutrients from the basal media with the 50% v/v saline addition. The use of saline excipient treatment will serve as an internal control for the quality of assay with <10% difference considered acceptable.

Evaluation of the assay as culture of CCRF-CEM T cells with 50% v/v RNSA treatment for 24 and 48 hours demonstrates that 48 hours is a suitable timepoint to show the inhibition of proliferation and decreased viability, as well as discerning lot-to-lot variability. Specifically, RNSA lots 082721 and 100821 demonstrates a significant decrease (p<0.05) in cell proliferation at 24 hours (52±1.4% 082721, 47±4.2% 100821) that further decreased at 48 hours (33±2.9% 082721, 31±3.8% 10081) (FIG. 1C). RNSA lot 101421 exhibited a similar level of significant decreased proliferation at both 24 and 48 hours, 67±1.8% vs 69±2.6% of the final cell count in the saline reference control group; hence not as potent as the previous two lots. However, the inability to further inhibit T cell proliferation with longer duration treatment highlights that the 48-hour timepoint allows for greater sensitivity in detecting potency differences of RNSA, and as such the 48-hour timepoint has been chosen for the purpose of this assay. Similarly, the significant pro-apoptotic properties of RNSA based on cell viability testing is demonstrated at the 48-hour timepoint at 56.7±1.7% (lot 082721), 53.6±2% (100821) and 88.7±2.3% (lot101421) as compared to 24 hours at 85±1.2% (lot082721), 78.2±3.3% (lot100821) and 94.4±1.7% (lot101421), congruent with the anti-proliferative effects (FIG. 49D).

FIG. 49A-D. Development of a T cell proliferation assay to assess the potency of RNSA biological candidate in acute lymphoblastic leukemia cell lines Jurkat and CCRF-CEM. Jurkat and CCRF-CEM T cell lines were utilized to assess the effect of RNSA lots (082721, 100821 and 101421) on inhibition of cellular proliferation (A) and percent viability (B) after 48 hours of treatment with the drug candidate compared to saline. Optimization of the T cell proliferation assay with the CCRF-CEM T lymphoblasts after culturing for 24 and 48 hours as assessed by fold change in proliferation compared to 24-hour timepoint of no treatment group (C) and percent viability (D). Statistical analysis represented by Two-way ANOVA with Šidák's multiple comparisons post-hoc test where &<0.05 represents comparison to saline (A, B); a<0.05 against NT, &<0.05 against saline 24 hrs, $<0.05 against saline at 48 hours, *<0.05 24 vs 48 hours. n=2-3. NT-no treatment/standard media, saline-0.9% Sodium Chloride, RNSA lots-082721, 100821, 101421.

Based on the robustness of the CCRF-CEM T cell proliferation assay described above, we evaluated the first 5 lots manufactured in a GMP environment at Signature Biologics facility. As stated above, the present expectation is that at a 1:1 dilution (50% v/v) of RNSA within the cell culture assay will yield a 50% inhibition T cell proliferation over 48 hours treatment compared to the vehicle control (saline, excipient solution). Also, to highlight the anti-proliferative properties of RNSAs according to the invention, the data is presented here as a percentage of inhibition normalized to saline control, as opposed to the percentage of proliferation (presented above).

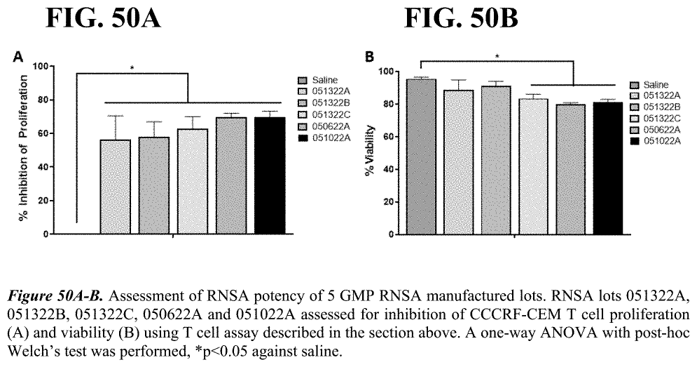

FIG. 50A-B contains an assessment of RNSA potency of additional 5 GMP manufactured lots. RNSA lots 051322A, 051322B, 051322C, 050622A and 051022A were assessed for inhibition of CCCRF-CEM T cell proliferation (A) and viability (B) as per T cell assay described in the section above. A one-way ANOVA with post-hoc Welch's test was performed, *p<0.05 against saline.

The potency of RNSAs according to the invention, stored at −20° C. fulfilled the requirement of 50% inhibition in all five GMP lots of RNSA (051322A, 051322B, 051322C, 050622A, 051022A) (FIG. 50A). Viability of the CCRF-CEM cells with RNSA treatment as a proxy measure of potential pro-apoptotic effects of activated T cells, is significantly decreased compared to saline only with 051322C, 050622A and 051022A lots. The viability of T cells is monitored for discovery efforts as potential by-product of anti-proliferative effects of RNSAs according to the invention, rather than a measure of critical quality attribute for the purpose of fulfilling the release criteria metric. Overall, the results above indicate that manufacture GMP lots fulfilled the release criteria metric of >50% inhibition of T cell proliferation, thereby verifying the biological activity of RNSA.

The T cell proliferation assay is currently employed for twenty-four-month aging study as proof of concept of using a cell based assay for testing storage condition and shelf life stability of biological candidate RNSA.

We claim:

1. A cell-free or substantially cell-free regenerative nonsteroidal anti-inflammatory composition suitable for therapeutic or prophylactic use comprising a therapeutically or prophylactically effective amount of an isolated cell-free or substantially cell-free placenta-derived extract obtained from placental tissue from one or more mammalian donors wherein such tissue has naturally or been induced to undergo lysis, preferably stress-induced lysis, by a mechanism selected from cellular stress, apoptosis, necrosis, anoikis, or non-apoptotic programmed cell death, wherein:
i. said placenta-derived extract comprises one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA;
ii. said composition is capable of inhibiting proliferation of activated T cells and is non-cytotoxic for one or more cells selected from stromal cells, mesenchymal stromal cells (MSCs), parenchymal cells, and tenocytes in a subject, in vivo, or in vitro.

2. The composition of claim 1, comprising one or more of the following:
(i) the placenta is selected from human, non-human primate, pig, sheep, horse, cow, dog, cat, rat, and mouse placenta, preferably human placenta;
(ii) the placental tissue is obtained from a single donor;
(iii) the placental tissue is obtained from more than one donor (pooled donor placental tissue sample);
(iv) the placenta comprises at least one placental tissue selected from amniotic membrane, chorion membrane, chorionic villus, umbilical cord, and Wharton's Jelly, preferably selected from at least one of amniotic membrane and/or chorion membrane′
(v) the at least one placental tissue comprises perinatal stromal cells (PSCs) and/or mesenchymal stromal cells (MSCs);
(vi) said composition is stable in solution at room temperature for at least eight weeks;
(vii) said composition is stable to lyophilization;
(viii) the T cells comprise CD4+, CD8+, CD4+/CD8+, CD11c+, CD11b+, and/or CD56+ T cells;
(ix) it is further capable of promoting proliferation of one or more cells selected from stromal cells, mesenchymal stromal cells (MSCs), parenchymal cells, and tenocytes in a subject, in vivo, or in vitro;
(x) it is capable of reducing expression of one or more pro-inflammatory cytokines from activated peripheral blood mononucleated cells (PBMCs) and/or activated T cells in a subject, in vivo, or in vitro, optionally wherein the one or more pro-inflammatory cytokines is selected from TNFα, NFκB, IL-17A, IL-6, and IFNγ;
(xi) it is capable of increasing cAMP production from activated T cells in a subject, in vivo, or in vitro;
(xii) it is modified by the addition of one or more other constituents, optionally non-actives or actives such as antibodies, cytokines, hormones, growth factors, drugs, antibiotics, analgesics, preservatives, pharmaceutically acceptable carriers and excipients, cells, e.g., autologous or allogeneic donor cells, e.g., immune cells, or any combination of the foregoing;
(xiii) it has been stored in a sealed vial at a temperature ranging from 4° C. to 40° C. for a time period ranging from 1 week to 24 weeks; or
(xiv) any combination of one or more of the foregoing.

3-4. (canceled)

5. A method for producing a cell-free or substantially cell-free regenerative nonsteroidal anti-inflammatory composition, comprising:
i. obtaining at least one placental tissue from at least one mammal selected from human, non-human primate, pig, sheep, horse, cow, dog, cat, rat, and mouse, wherein the at least one placental tissue is selected from amniotic membrane, chorion membrane, chorionic villus, umbilical cord, and Wharton's Jelly, and wherein the at least one placental tissue comprises perinatal stromal cells (PSCs);
ii. optionally isolating the PSCs from said placental tissue and culturing the PSCs in at least one cell culture medium;
iii. permitting stress-induced lysis of said placental tissue and PSCs comprised therein and/or permitting stress-induced lysis of PSCs isolated therefrom to naturally occur and/or inducing stress-induced lysis of said placental tissue and PSCs comprised therein and/or inducing stress-induced lysis of PSCs isolated therefrom to produce a placenta-derived extract; and
iv. separating the placenta-derived extract or a portion thereof from the cells and tissue, for example, by decantation, centrifugation, and/or filtration;
thereby producing the cell-free or substantially cell-free regenerative nonsteroidal anti-inflammatory composition.

6. The method of claim 5, comprising at least one of the following:
(i) the PSCs comprise mesenchymal stromal cells (MSCs);
(ii) the mammal is a human or non-human primate;
(iii) the method further comprises conducting one or more screening assays to assess the effects of the isolated placenta-derived extract or one or more portions thereof on the proliferation of activated T cells and/or the proliferation of one or more cells selected from stromal cells, mesenchymal stromal cells (MSCs), parenchymal cells, and tenocytes and/or on the expression of pro-inflammatory cytokines and/or the expression of anti-inflammatory cytokines in a mammalian subject, in vivo, or in vitro;
(iv) different portions of the isolated placenta-derived extract are screened in order to assess potency;
(v) inducing stress-induced lysis comprises serum deprivation, nutrient deprivation, and/or hypoxia;
(vi) stress-induced lysis is induced by the following:
a. contacting the placental tissue with a non-cell culture medium in a ratio ranging from about 1 mL non-cell culture medium per 1 gram of placental tissue to about 100 mL non-cell culture medium per 1 gram of placental tissue, preferably in a ratio of about 10 mL non-cell culture medium per 1 g of placental tissue; and
b. incubating the placental tissue in the non-cell culture medium in an air-tight environment at a temperature ranging from about 4° C. to about 42° C., preferably at about 37° C., for about 2 days to about 12 days, more preferably for about 10 days, wherein the incubating optionally comprises agitation, for example, at about 90 rpm;
(vii) the method further comprises isolating the placental tissue PSCs and culturing the PSCs in at least one cell culture medium prior to inducing stress-induced lysis;
(viii) stress-induced lysis is effected by:
a. replacing the at least one cell culture medium with a non-cell culture medium; and
b. incubating the cultured PSCs in the non-cell culture medium in an air-tight environment at a temperature ranging from about 4° C. to about 42° C., preferably at about 37° C., for about 3 days to about 5 days, preferably for about 4 days, wherein the incubating optionally comprises agitation;
(ix) the cultured PSCs are cultured to at least 80% confluence;
(x) the non-cell culture medium comprises saline solution;
(xi) the non-cell culture medium comprises saline solution that comprises 0.9% NaCl;
(xii) the non-cell culture medium comprises saline solution that comprises phosphate-buffered saline (PBS);
(xiii) the air-tight environment prevents gas exchange, thereby inducing a hypoxic environment;
(xiv) the method further comprises washing the placental tissue with phosphate-buffered saline (PBS) prior to inducing stress-induced lysis;
(xv) the method further comprises mincing the placental tissue prior to inducing stress-induced lysis;
(xvi) the method further comprises washing the cultured MSCs with phosphate-buffered saline (PBS) prior to inducing stress-induced lysis;
(xvii) the method further comprises contacting the placental tissue with one or more antimicrobial agents;
(xviii) the centrifugation comprises centrifugation at about 10,000×g for about 30 minutes;
(xix) the filtration comprises filtration through a 0.45 μm membrane;
(xx) the filtration comprises filtration through a 0.2 μm membrane, i.e. sterile filtration;
(xxi) the filtration comprises filtration through a 30 KDa MWCO membrane, a 10 KDa MWCO membrane, a 5 KDa MWCO membrane, a 3 KDa MWCO membrane, and/or a 2 KDa MWCO membrane; or
(xxii) said cell-free or substantially cell-free regenerative nonsteroidal anti-inflammatory composition is stored at a temperature ranging from 4° C. to 40° C. for a time period ranging from 1 week to 24 weeks;
(xxiii) any combination of one or more of (i) to (xxii).

7-11. (canceled)

12. A cell-free or substantially cell-free regenerative nonsteroidal anti-inflammatory composition produced by the method of claim 5.

13. The composition of claim 1, which:
(i) comprises one or more eicosanoids optionally selected from 6kPGF1α, TXB2, PGF2α, PGE2, PGA2, LTB4, 5oxoETE, 5HETE, 11HETE, 12HETE, 15HETE, 20HETE, 5,6DHET, 8,9DHET, 11,12DHET, 14,15DHET, 9HODE, 13HODE, and AA;
(ii) is capable of inhibiting proliferation of activated T cells in a subject, in vivo, or in vitro, wherein the T cells are CD4+, CD8+, CD4+/CD8+, CD11c+, CD11b+, and/or CD56+ T cells;
(iii) is non-cytotoxic for one or more cell types selected from stromal cells, mesenchymal stromal cells (MSCs), parenchymal cells, and tenocytes, in a subject, in vivo, or in vitro;
(iv) is capable of promoting proliferation of one or more cell types selected from stromal cells, mesenchymal stromal cells (MSCs), parenchymal cells, and tenocytes, in a subject, in vivo, or in vitro;
(v) is capable of reducing expression of one or more pro-inflammatory cytokines from activated peripheral blood mononucleated cells (PBMCs) and/or activated T cells in a subject, in vivo, or in vitro;
(vi) the one or more pro-inflammatory cytokines is selected from TNFα, NFκB, IL17A, IL-6, and IFNγ;
(vii) is capable of increasing cAMP production from activated T cells in a subject, in vivo, or in vitro;
(viii) is stable in solution at room temperature for at least eight weeks;
(ix) is stable to lyophilization;
(x) is modified by the addition of one or more other constituents, optionally non-actives or actives such as antibodies, cytokines, hormones, growth factors, drugs, antibiotics, analgesics, preservatives, pharmaceutically acceptable carriers and excipients, cells, e.g., autologous or allogeneic donor cells, e.g., immune cells, or any combination of the foregoing; or
(xi) any combination of (i) to (x).

14. A method of treatment or prevention of at least one inflammatory condition or disease or at least one symptom associated therewith, comprising administering a therapeutically or prophylactically effective amount of the cell-free or substantially cell-free regenerative nonsteroidal anti-inflammatory composition of claim 1 to a subject in need thereof.

15. The method of treatment or prevention of claim 14, wherein
(i) the at least one inflammatory condition or disease is an acute or chronic condition associated with inflammation, e.g., an acute or chronic autoimmune disease associated with acute or chronic inflammation, optionally a viral or bacterial or fungal infection associated with acute or chronic inflammation, further optionally a hepatitis virus, ZIKA virus, herpes, papillomavirus, influenza virus, or coronavirus, further optionally COVID-19 or SARS;
(ii) the at least one inflammatory condition or disease is an acute inflammatory condition or disease optionally a viral infection associated with acute inflammation, further optionally a coronavirus infection, e.g., COVID-19 or SARS;
(iii) the at least one inflammatory condition or disease is selected from pneumonia, single or multiple organ failure or dysfunction, sepsis, cytokine storm, fever, neurological dysfunction or impairment, loss of taste or smell, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, allergy, asthma, glomerulonephritis, pancreatitis, hepatitis, inflammatory arthritis, gout, multiple sclerosis, psoriasis, Acute Respiratory Distress Syndrome (ARDS), wound healing, diabetic ulcers, non-healing wounds, lupus, and other autoimmune diseases associated with acute or chronic inflammation;
(iv) the symptoms associated with the inflammatory condition include one or more of pneumonia, cytokine storm, single or multiple organ failure, fibrosis, impaired respiratory function such as acute or chronic respiratory distress syndrome, fever, impaired cardiac function, impaired lung function, impaired liver function, impaired taste or smell, and impaired neurological function;
(v) the subject has pneumonia, optionally Covid-19-associated pneumonia and/or a pneumonia associated with another virus, e.g., influenza or another coronavirus, and/or a pneumonia associated with a fungus or bacterium;
(vi) the subject has ophthalmic inflammation which comprises one or more of corneal regeneration, corneal wound healing, corneal melting, dry eye, ocular infection, eyelid sty, and autoimmune-associated peripheral ulcerative keratitis;
(vii) the subject has fibrosis that comprises one or more of pulmonary fibrosis, cystic fibrosis, idiopathic pulmonary fibrosis, interstitial pulmonary fibrosis, radiation-induced lung injury, liver fibrosis, bridging fibrosis of the liver, cirrhosis, glial scar, arterial stiffness, arthrofibrosis, Crohn's disease, Dupuytren's contracture, keloid fibrosis, Mediastinal fibrosis, Myelofibrosis, Myocardial fibrosis, Peyronie's disease, Nephrogenic systemic fibrosis, Progressive massive fibrosis, pneumoconiosis, Retroperitoneal fibrosis, stromal fibrosis, Scleroderma, systemic sclerosis, chronic obstructive pulmonary disease (COPD), asthma, and adhesive capsulitis;
(viii) the subject has gastrointestinal inflammation which comprises one or more of inflammatory bowel disease (IBD), Crohn's disease, ulcerative colitis, irritable bowel syndrome (IBS), and Celiac disease;
(ix) the subject has ophthalmic inflammation which is associated with keratoconjunctivitis sicca;
(x) the subject has dermatologic inflammation associated with eczema or psoriasis;
(xi) the at least one autoimmune disease is selected from the group consisting of Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome, (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndrome type I, Polyglandular syndrome type II, Polyglandular syndrome type III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease;
(xii) the effective amount comprises one or more doses of the composition, wherein each dose is selected from a list of dosage ranges comprising 0.01-5 mL, preferably 1 mL administered locally to locations such as tendons, ligaments, and joints; 0.01-2 mL, preferably 0.1 mL administered to each eye for topical eye indications; 5-100 mL, preferably 8 mL administered systemically; 0.5-5 mL, preferably 3 mL administered as an inhaled mist;
(xiii) the composition is administered by one or more of injection, optionally intravenous (V) or subcutaneous (SC) administration, nebulization, and eye drops;
(xiv) the subject is selected from a human, non-human primate, pig, sheep, horse, cow, dog, cat, rat, and mouse, preferably human;
(xv) it further comprises the administration of at least one other active, e.g., an anti-inflammatory agent such as an anti-inflammatory antibody or anti-inflammatory fusion protein, an antiviral agent, an antibacterial agent, an antifungal agent, an analgesic, an anti-congestive agent, an anti-fever agent, or a combination of any of the foregoing;
(xvi) the subject has been diagnosed with or is suspected of having a coronavirus infection, optionally COVID-19;
(xvii) the subject has been diagnosed with a coronavirus infection, optionally COVID-19, and is on a respirator, has Acute Respiratory Distress Syndrome (ARDS), and/or is experiencing respiratory difficulties;
(xviii) the subject has been diagnosed with or suspected of having a coronavirus infection, optionally COVID-19, and optionally the subject comprises one or more risk factors that place the subject at higher risk for morbidity or a poor treatment outcome, e.g., age over 55 years, obesity, diabetes, cardiac problem or condition, respiratory condition, optionally asthma, COPD, cystic fibrosis, is a smoker, is a heavy drinker, has lupus, has elevated blood pressure, has cancer, receives chemotherapy, has (chronic) kidney disease and/or is on dialysis, or any combination of the foregoing; or
(xix) any combination of (i) to (xviii).

16-26. (canceled)

27. A method for producing a cell-free or substantially cell-free regenerative nonsteroidal anti-inflammatory composition, comprising:
i. obtaining at least one placental tissue from at least one mammal selected from human, non-human primate, pig, sheep, horse, cow, dog, cat, rat, and mouse, wherein the at least one placental tissue is selected from amniotic membrane, chorion membrane, chorionic villus, umbilical cord, and Wharton's Jelly, and wherein the at least one placental tissue comprises perinatal stromal cells (PSCs);
ii. optionally isolating the PSCs from said placental tissue and culturing the PSCs in at least one cell culture medium;
iii. permitting stress-induced lysis of said placental tissue and PSCs comprised therein and/or permitting stress-induced lysis of PSCs isolated therefrom to naturally occur and/or inducing stress-induced lysis of said placental tissue and PSCs comprised therein and/or inducing stress-induced lysis of PSCs isolated therefrom to produce a placenta-derived extract; and
iv. separating the placenta-derived extract or a portion thereof from the cells and tissue, for example, by decantation, centrifugation, and/or filtration;
v. storing the placenta-derived extract or a portion thereof in a sealed vial at a temperature ranging from 4° C. to 40° C. for a time period ranging from 1 week to 24 weeks; thereby producing the cell-free or substantially cell-free regenerative nonsteroidal anti-inflammatory composition.

28. A cell-free or substantially cell-free regenerative nonsteroidal anti-inflammatory composition produced by the method of claim 27.

29. A method of treatment or prevention of at least one infection, cancer, inflammatory or autoimmune condition or disease associated wherein elevated TNFα is associated with the disease pathology or the side effects of a treatment regimen, comprising administering a therapeutically or prophylactically effective amount of a cell-free or substantially cell-free regenerative nonsteroidal anti-inflammatory composition or a cell-free filtrate derived therefrom of claim 1, optionally a fraction comprising molecules <1 KDa, to a subject in need thereof.

30. The method of claim 29, wherein
(i) said inflammatory or autoimmune condition or disease or side effects of a treatment regimen comprises one or more of rheumatoid arthritis, psoriatic arthritis, Crohn's disease, fatty liver disease, NASH, asthma, an inflammatory metabolic disorder, optionally type 1 or type 2 diabetes or obesity, inflammatory bowel disease, noninfectious uveitis, sepsis, cytokine storm, cancer, side effects associated with cancer therapy, optionally radiotherapy, chemotherapy, hormone and/or biologic therapy, side effects associated with transplanted cells, tissues and/or or organ, optionally bone marrow transplant, inflammation associated with an acute or chronic viral condition, a neuroinflammatory condition, optionally multiple sclerosis, Alzheimer's disease, migraine, Neuromyelitis optica (NMO), Anti-myelin oligodendrocyte glycoprotein antibody disorder (MOG), Autoimmune encephalitis, Transverse Myelitis, Optic neuritis, neurosarcoidosis, Parkinson's disease, or schizophrenia;
(ii) TNFα levels are detected in the treated subject before, during and/or after treatment, optionally by detecting TNFα levels in one or more samples obtained from the subject;
(iii) treatment is only effected if TNFα levels are elevated in the subject prior to treatment;
(iv) treatment efficacy is monitored after treatment is initiated based on the [reducing] effect of the treatment on TNFα levels, which optionally is determined by detecting TNFα levels in one or more samples obtained from the subject;
(v) any combination of (i) to (iv).

31-33. (canceled)

34. A method of activating a Peroxisome proliferator-activated receptors (PPARs), optionally PPARα, PPARβ/δ or (PPARδ), and/or PPARγ in a subject in need thereof, optionally one with a cancer, infectious, autoimmune or inflammatory condition, by administering a therapeutically or prophylactically effective amount of a cell-free or substantially cell-free regenerative nonsteroidal anti-inflammatory composition or a cell-free filtrate derived therefrom of claim 1, optionally a fraction comprising molecules <1 KDa.

35. The method of claim 34, wherein
(i) the subject has a condition associated with reduced or inhibited activities associated with the activation of Peroxisome proliferator-activated receptors (PPARs), optionally PPARα, PPARβ/δ or (PPARδ), and/or PPARγ;
(ii) activities elicited by Peroxisome proliferator-activated receptors (PPARs), optionally PPARα, PPARβ/δ or (PPARδ), and/or PPARγ are detected in vivo and/or are detected in one or more samples obtained from the subject before, during and/or after treatment;
(iii) Peroxisome proliferator-activated receptor (PPAR) activity, optionally PPARα, PPARβ/δ or (PPARγ), and/or PPARγ activity, is detected in vivo and/or are detected in vitro in one or more samples obtained from the subject after treatment has initiated to assess treatment efficacy;
(iv) Peroxisome proliferator-activated receptor (PPAR) activity, optionally PPARγ, PPARβ/δ or (PPARγ), and/or PPARγ activity, is detected using one or more methods such as an antibody-based method, optionally immunohistochemistry, immunofluorescence or Western blotting, measurement of PPARγ target gene expression using PCR or a PPAR response element (PPRE) luciferase assay, measurement of PPARγ transcriptional activity, a DNA binding immunoassay that measures the amount of free PPARγ in nuclear extracts or Serum PPARγ Activity Assay (SPAA);
(v) the subject has a metabolic disorder, hypertrophic obesity, insulin-resistance, acute or chronic inflammatory kidney disease, cancer, non-alcoholic fatty liver disease (NAFLD), non-alcoholic steatohepatitis (NASH), fibrosis, Alzheimer's disease (AD), dyslipidemia, hyperglycemia, type 1 or 2 diabetes, a lung disease such as COPD or asthma, a neurodegenerative disorder, obesity, hypertension, atherosclerosis, a cardiovascular condition, vascular injury, heart attack, myocarditis, pericarditis, contractile dysfunction, stroke, hypertension, a neurodegenerative condition, diabetic nephropathy, retinopathy, inflammatory bowel disease, ulcerative colitis, or Crohn's disease (CD); or
(vi) any combination of (i) to (v).

36-39. (canceled)

40. A method of treatment or prevention of at least one condition or disease wherein reactive oxygen species are associated with the disease pathology or wherein reactive oxygen species are side effects associated with a treatment regimen, optionally chemotherapy, radiotherapy, gene therapy, cell therapy, cell, organ and/or tissue transplantation, comprising administering a therapeutically or prophylactically effective amount of a cell-free or substantially cell-free regenerative nonsteroidal anti-inflammatory composition or a cell-free filtrate derived therefrom of any one of the foregoing claims, optionally a fraction comprising molecules <1 KDa, to a subject in need thereof.

41. The method of claim 40, wherein
(i) the condition or disease comprises a respiratory condition such as asthma, chronic obstructive pulmonary diseases, inflammatory bowel disease, neurodegenerative disorders such as Alzheimer's disease (AD), Huntington's disease (HD), amyotrophic lateral sclerosis (ALS), Parkinson's disease (PD), and spinocerebellar ataxia (SCA), cardiovascular diseases such as atherosclerosis, cardiac hypertrophy, ischemic-reperfusion injury, myocyte apoptosis, heart failure, cancers, optionally lung, breast, tongue, gastric, larynx, colon, rectal, lung and prostate cancers; further optionally cancers wherein ROS contribute to resistance to therapies, metastasis, aberrant angiogenesis and normal or aberrant aging;
(ii) the levels of reactive oxygen species (ROS) are detected in the subject or in one or more samples obtained from the subject before, during or after therapy;
(iii) the levels of reactive oxygen species (ROS) are detected in the subject or in one or more samples obtained from the subject after therapy has been initiated, optionally to detect treatment efficacy;
(iv) the levels of reactive oxygen species (ROS) are detected using one or more techniques, optionally colorimetric assays, immunoblotting, immunofluorescence, flow cytometry, optionally using the FL1 channel (green fluorescence) or FITC channel, by fluorescence microscopy, by use of carboxy-H2DCFDA and by detecting ROS-caused alteration of macromolecules using immunohistochemistry; or
(v) any combination of the foregoing.

42-44. (canceled)

45. A method of reducing T cell proliferation and/or T cell activation in a subject in need thereof, by administering a therapeutically or prophylactically effective amount of a cell-free or substantially cell-free regenerative nonsteroidal anti-inflammatory composition or a cell-free filtrate derived therefrom of any one of the foregoing claims, optionally a fraction comprising molecules <1 KDa.

46. The method of claim 45, wherein
the subject has an inflammatory or autoimmune condition wherein T cells contribute to the disease pathology;
(ii) the treated condition comprises rheumatoid arthritis (RA), systemic lupus erythematosus (SLE), ITP, CD, IBD, myositis, psoriasis, psoriatic arthritis, vasculitis, scleroderma, type 1 or type 2 diabetes, hypothyroidism, Addison's disease, sepsis, cytokine storm, a neurodegenerative condition, neurological dysfunction or impairment, cardiac dysfunction, pulmonary dysfunction, liver dysfunction, acute or chronic respiratory dysfunction, graft versus host disease (GVHD), cardiomyopathy, vasculitis, fibrosis, ophthalmic inflammation, dermatologic inflammation, gastrointestinal inflammation, tendinopathies, asthma, glomerulonephritis, pancreatitis, hepatitis, inflammatory arthritis, gout, multiple sclerosis, Acute Respiratory Distress Syndrome (ARDS), wound healing, diabetic ulcers, non-healing wounds, lupus, or other autoimmune diseases associated with T cell mediated acute or chronic inflammation;
(iii) T cell proliferation and/or T cell activation is detected in the subject or in one or more samples obtained from the subject prior, during or after treatment;
(iv) T cell proliferation and/or T cell activation is detected in the subject or in one or more samples obtained from the subject after treatment in order to assess treatment efficacy; or
(v) any combination of (i) to (iv).

47-49. (canceled)
